SEQUENCE LISTING (50 sequences)

SEQ ID NO: 1 - protein, 842 aa, Arabidopsis thaliana
MEMAVANHRERSSDSMNRHLDSSGKYVRYTAEQVEALERVYAECPKPSSLRRQQLIRECSILANIEPKQIKVWFQNRRCRDKQRKEASRLQSVNRKLSAMNKLLMEENDRLQKQVSQLVCENGYMKQQLTTVVNDPSCESVVTTPQHSLRDANSPAGLLSIAEETLAEFLSKATGTAVDWVQMPGMKPGPDSVGIFAISQRCNGVAARACGLVSLEPMKIAEILKDRPSWFRDCRSLEVFTMFPAGNGGTIELVYMQTYAPTTLAPARDFWTLRYTTSLDNGSFVVCERSLSGSGAGPNAASASQFVRAEMLSSGYLIRPCDGGGSIIHIVDHLNLEAWSVPDVLRPLYESSKVVAQKMTISALRYIRQLAQESNGEVVYGLGRQPAVLRTFSQRLSRGFNDAVNGFGDDGWSTMHCDGAEDIIVAINSTKHLNNISNSLSFLGGVLCAKASMLLQNVPPAVLIRFLREHRSEWADFNVDAYSAATLKAGSFAYPGMRPTRFTGSQIIMPLGHTIEHEEMLEVVRLEGHSLAQEDAFMSRDVHLLQICTGIDENAVGACSELIFAPINEMFPDDAPLVPSGFRVIPVDAKTGDVQDLLTANHRTLDLTSSLEVGPSPENASGNSFSSSSSRCILTIAFQFPFENNLQENVAGMACQYVRSVISSVQRVAMAISPSGISPSLGSKLSPGSPEAVTLAQWISQSYSHHLGSELLTIDSLGSDDSVLKLLWDHQDAILCCSLKPQPVFMFANQAGLDMLETTLVALQDITLEKIFDESGRKAICSDFAKLMQQGFACLPSGICVSTMGRHVSYEQAVAWKVFAASEENNNNLHCLAFSFVNWSFV

SEQ ID NO: 2 - protein, 839 aa, Oryza sativa
MAAAVAMRGSSSDGGGYDKVSGMDSGKYVRYTPEQVEALERVYADCPKPTSSRRQQLLRECPILANIEPKQIKVWFQNRRCRDKQRKESSRLQAVNRKLTAMNKLLMEENERLQKQVSQLVHENAHMRQQLQNTPLANDTSCESNVTTPQNPLRDASNPSGLLSIAEETLTEFLSKATGTAIDWVQMPGMKPGPDSVGIVAISHGCRGVAARACGLVNLEPTKVVEILKDRPSWFRDCRNLEVFTMIPAGNGGTVELVYTQLYAPTTLVPARDFWTLRYTTTMEDGSLVVCERSLSGSGGGPSAASAQQYVRAEMLPSGYLVRPCEGGGSIVHIVDHLDLEAWSVPEVLRPLYESSRVVAQKMTTAALRHIRQIAQETSGEVVYALGRQPAVLRTFSQRLSRGFNDAISGFNDDGWSIMGGDGVEDVVIACNSTKKIRSNSNAGIAFGAPGGIICAKASMLLQSVPPAVLVRFLREHRSEWADYNIDAYLASTLKTSACSLTGLRPMRFSGSQIIIPLAHTVENEEILEVVRLEGQPLTHDEALLSRDIHLLQLCTGIDEKSVGSSFQLVFAPIDDFPDETPLISSGFRVIPLDMKTDGASSGRTLDLASSLEVGSATAQASGDASADDCNLRSVLTIAFQFPYELHLQDSVAAMARQYVRSIVSAVQRVSMAISPSQTGLNAGQRIISGFPEAATLARWVCQSYHYHLGVELLSQSDGDAEQLLKMLWHYQDAILCCSFKEKPVFTFANKAGLDMLETSLVALQDLTLDRIFDEPGKEALFSNIPKLMEQGHVYLPSGVCMSGMGRHVSFDQAVAWKVLAEDSNVHCLAFCFVNWSFV

SEQ ID NO: 3 - protein, 840 aa, Oryza sativa
MAAAVAMRSGSGSDGGGGGYDKAGMDSGKYVRYTPEQVEALERVYAECPKPSSSRRQQLLRDCPILANIEPKQIKVWFQNRRCRDKQRKEASRLQAVNRKLTAMNKLLMEENERLQKQVSQLVHENAYMKQQLQNPSLGNDTSCESNVTTPQNPLRDASNPSGLLTIAEETLTEFLSKATGTAVDWVPMPGMKPGPDSFGIVAVSHGCRGVAARACGLVNLEPTKIVEILKDRPSWFRDCRSLEVFTMFPAGNGGTIELVYMQMYAPTTLVPARDFWTLRYTTTMEDGSLVVCERSLSGSGGGPSTASAQQFVRAEMLPSGYLVRPCEGGGSIVHIVDHLDLEAWSVPEVLRPLYESSRVVAQKMTTAALRHIRQIAQETSGEVVYALGRQPAVLRTFSQRLSRGFNDAISGFNDDGWSVMGGDGIEDVIIACNAKKVRNTSTSANAFVTPGGVICAKASMLLQSVPPAVLVRFLREHRSEWADYNFDAYSASSLKTSSCSLPGLRPMRFSGSQIIMPLAHTVENEEILEVVRLEGQALTHDDGLMSRDIHLLQLCTGIDEKSMGSCFQLVSAPIDELFPDDAPLISSGFRVIPLDMKTDGTPAGRTLDLASSLEVGSTAQPTGDASMDDCNLRSVLTIAFQFPYEMHLQDSVATMARQYVRSIVSSVQRVSMAISPSRSGLNAGQKIISGFPEAPTLARWICQSYQFHLGVELLRQADDAGEALLKMLWDYEDAILCCSFKEKPVFTFANEMGLNMLETSLVALQDLSLDKIFDEAGRKALYNEIPKLMEQGYVYLPGGVCLSGMGRHVSFEQAVAWKVLGEDNNVHCLAFCFVNWSFV

SEQ ID NO: 4 - protein, 840 aa, Zea mays
MAAAVAMRGGSSDSGGFDKVPGMDSGKYVRYTPEQVEVLERLYIDCPKPSSSRRQQLLRECPILSNIEPKQIKVWFQNRRCRDKQRKESSRLQAVNRRLTAMNKLLMEENERLQKQVSQLVHENAHMRQQLQNTSLANDTSCESNVTAPPNAIRDASNPSGLLAIAEETFTEFLSKATGTAIDWVQMPGMKPGPDSVGIVAISHGCRGVAARACGLVNLEPTKVIEILKDRPSWFRDCRSLEVFTMFPAGNGGTIELIYMQMYAPTTLVPARDFWTLRYTTTMEDGSLVVCERSLTGSGGGPNAASAQQFVRAEMLPSGYLVRPCEGGGSIVHIVDHLDLEAWSVPEVLRPLYESSRVVAQKMTTVALRHLRQIAQETSGEVVYALGRQPAVLRTFSQRLSRGFNDAISGFNDDGWSVMGGDGIEDVVVACNSTKKIRNNSNAGITFGAPGGIICAKASMLLQSVPPAVLVRFLREHRSEWADYNIDAYLASSLKTSACSLPGLRPMRFSGGQMIMPLAHTVENEEILEVVRLEGQPLTHDEALLSRDIHLLQLCTGIDEKSVGSSFQLVFAPIDEHFPDDAPLISSGFRVIPLDMKTDGVSSGRTLDLASSLDVGSAAPQASGDAPPDDCNLRSVLTIAFQFPYEMHLQDSVATMARQYVRSVVSAVQRVSMAISPSQSGLNAGQRMLSGFPEAATLARWVCQSYHYHLGVELLNQSDEAGEALLKMLWHHPDAVLCCSFKEKPMFTFANKAGLDMLETSLIALQDLTLDKIFDESGRKAIFSDISKLMEQGYAYLPSGVCMSGMGRHVSFDQAVAWKVLGEDSSVHCLAFCFVNWSFV

SEQ ID NO: 5 - DNA, 2523 bp, Zea mays
atggctgcagcggtggcgatgcgagggggaagcagcgacagcggaggatttgacaaggttcccgggatggactcggggaaatacgtgcgctacaccccggagcaagttgaggtgctcgagcggctctacatcgattgccccaagccaagctcctcacggcgccagcaactgctgcgcgagtgtcctatactctccaacattgagccgaagcagatcaaggtctggttccagaaccgaaggtgccgcgataagcagcggaaggagtcttcgcggcttcaggctgtcaacagaaggctgacggcaatgaacaagcttcttatggaagagaatgagcgtcttcagaagcaagtctctcagttggttcatgaaaatgcgcacatgcggcagcagctgcagaacacttcattggccaatgatacaagctgtgaatcaaatgtcactgcccctccaaacgctataagggatgcaagtaacccttctggactccttgcgattgcggaggagaccttcacagagttcctctcaaaggctactgggacagctattgattgggtccagatgcctgggatgaagcctggtccggattcggttggtattgtggccatttcgcatggttgccgtggcgttgctgcccgcgcctgtggtttggtgaatctagaaccaacaaaagtcatagaaatcttgaaagatcgtccctcttggttccgtgattgtcgaagtctggaagtgtttacaatgtttccagctggaaatgggggaacaattgaacttatctacatgcagatgtacgccccaacaactttagttcctgcacgtgacttttggacgttgcgatacacgacaacaatggaagatggcagccttgtggtctgtgagagatccttgactggttctggaggcggtccaaatgcagcttctgcacagcaatttgttagggctgagatgcttccaagtgggtatttagtgcgcccatgcgaaggtggaggttcgattgtgcatatagtggaccatctagatctcgaggcatggagtgttcctgaagtgcttcgaccactatatgagtcttctagagtagttgctcagaaaatgactactgtggcactgcgccaccttagacaaattgctcaagaaacaagtggagaagtagtgtatgccttgggaaggcaacctgcagttctacggacctttagtcaaagactaagcagggggtttaatgatgccattagtggtttcaacgatgatggttggtctgtaatggggggagatggcattgaagacgttgttgttgcttgcaactcaaccaagaaaattaggaataatagcaatgctgggattacatttggagcgcctggaggcattatttgtgcgaaggcatccatgttactgcagagcgtcccaccggcagtactggtccgatttctgagggagcatagatctgaatgggctgattacaatattgatgcgtatttggcttcatcactgaagaccagtgcgtgctcacttcctgggttgcgacccatgagattttctggggggcagatgatcatgccacttgctcacacagtggagaacgaggagattcttgaagttgtccgccttgaaggacaacctcttactcatgatgaagcccttctttcaagggacatccaccttctccagctttgcactggaatagatgagaaatctgtgggttcctccttccagcttgtgtttgcaccaattgatgagcattttccggatgatgctccattgatttcctctggctttcgtgtcataccacttgatatgaaaacagatggtgtatcctctggtaggacgctagatttggcatctagtcttgatgtgggttctgctgcaccccaagcctcaggggatgcacctccagatgactgtaatttgagatctgtgctgacaatcgcctttcaattcccttatgagatgcaccttcaggacagtgttgctactatggcccgtcaatatgtgcgtagtgttgtttctgctgtgcaaagggtgtcgatggctatatctccctcccaatctggtctaaatgctgggcagaggatgctttctggcttccctgaagctgccacacttgctagatgggtttgccagagttatcactaccatctaggtgtggaattactcaatcaatcagatgaagctggtgaagcattgttgaaaatgctctggcatcatccagatgctgttctgtgctgctcttttaaggagaaacctatgtttacgtttgcaaacaaggcagggctcgacatgttagaaacatctcttattgcgctgcaagacctgacgctagacaagatcttcgacgagtcaggaaggaaagcaatattctcagatatctcaaaactaatggaacagggctacgcgtacctgccgtcgggcgtgtgcatgtcaggaatgggtcggcatgtctcattcgaccaagctgtagcgtggaaggtgctcggcgaggacagcagcgtccactgcctggcgttctgcttcgtcaactggtccttcgtttga

SEQ ID NO: 6 - DNA, 2776 bp, Zea mays
cgcgagatttgcgcggctacgattctgcgttgccgagggagcaagggaaccaatggctgcagcggtggcgatgcgagggggaagcagcgacagcggaggatttgacaaggttcccgggatggactcggggaaatacgtgcgctacaccccggagcaagttgaggtgctcgagcggctctacatcgattgccccaagccaagctcctcacggcgccagcaactgctgcgcgagtgtcctatactctccaacattgagccgaagcagatcaaggtctggttccagaaccgaaggtgccgcgataagcagcggaaggagtcttcgcggcttcaggctgtcaacagaaggctgacggcaatgaacaagcttcttatggaagagaatgagcgtcttcagaagcaagtctctcagttggttcatgaaaatgcgcacatgcggcagcagctgcagaacacttcattggccaatgatacaagctgtgaatcaaatgtcactgcccctccaaacgctataagggatgcaagtaacccttctggactccttgcgattgcggaggagaccttcacagagttcctctcaaaggctactgggacagctattgattgggtccagatgcctgggatgaagcctggtccggattcggttggtattgtggccatttcgcatggttgccgtggcgttgctgcccgcgcctgtggtttggtgaatctagaaccaacaaaagtcatagaaatcttgaaagatcgtccctcttggttccgtgattgtcgaagtctggaagtgtttacaatgtttccagctggaaatgggggaacaattgaacttatctacatgcagatgtacgccccaacaactttagttcctgcacgtgacttttggacgttgcgatacacgacaacaatggaagatggcagccttgtggtctgtgagagatccttgactggttctggaggcggtccaaatgcagcttctgcacagcaatttgttagggctgagatgcttccaagtgggtatttagtgcgcccatgcgaaggtggaggttcgattgtgcatatagtggaccatctagatctcgaggcatggagtgttcctgaagtgcttcgaccactatatgagtcttctagagtagttgctcagaaaatgactactgtggcactgcgccaccttagacaaattgctcaagaaacaagtggagaagtagtgtatgccttgggaaggcaacctgcagttctacggacctttagtcaaagactaagcagggggtttaatgatgccattagtggtttcaacgatgatggttggtctgtaatggggggagatggcattgaagacgttgttgttgcttgcaactcaaccaagaaaattaggaataatagcaatgctgggattacatttggagcgcctggaggcattatttgtgcgaaggcatccatgttactgcagagcgtcccaccggcagtactggtccgatttctgagggagcatagatctgaatgggctgattacaatattgatgcgtatttggcttcatcactgaagaccagtgcgtgctcacttcctgggttgcgacccatgagattttctggggggcagatgatcatgccacttgctcacacagtggagaacgaggagattcttgaagttgtccgccttgaaggacaacctcttactcatgatgaagcccttctttcaagggacatccaccttctccagctttgcactggaatagatgagaaatctgtgggttcctccttccagcttgtgtttgcaccaattgatgagcattttccggatgatgctccattgatttcctctggctttcgtgtcataccacttgatatgaaaacagatggtgtatcctctggtaggacgctagatttggcatctagtcttgatgtgggttctgctgcaccccaagcctcaggggatgcacctccagatgactgtaatttgagatctgtgctgacaatcgcctttcaattcccttatgagatgcaccttcaggacagtgttgctactatggcccgtcaatatgtgcgtagtgttgtttctgctgtgcaaagggtgtcgatggctatatctccctcccaatctggtctaaatgctgggcagaggatgctttctggcttccctgaagctgccacacttgctagatgggtttgccagagttatcactaccatctaggtgtggaattactcaatcaatcagatgaagctggtgaagcattgttgaaaatgctctggcatcatccagatgctgttctgtgctgctcttttaaggagaaacctatgtttacgtttgcaaacaaggcagggctcgacatgttagaaacatctcttattgcgctgcaagacctgacgctagacaagatcttcgacgagtcaggaaggaaagcaatattctcagatatctcaaaactaatggaacagggctacgcgtacctgccgtcgggcgtgtgcatgtcaggaatgggtcggcatgtctcattcgaccaagctgtagcgtggaaggtgctcggcgaggacagcagcgtccactgcctggcgttctgcttcgtcaactggtccttcgtttgacacgccatcccatggcctgtgtgatgagcaggccaatttttttgtatggtgatcccaatatggggaaaccatcagctgtgacagaaggctttatgttgcatgcactagttgtacgtcttgaatcgagtgtatttatgaagaaattggatactgcgcaaagtctggtgccacttacgtttgactaaaaaaaaaaaaaaaaaa

SEQ ID NO: 7 - protein, 430 aa, Lycopersicon esculentum
SSLRRQQLIRECHILSNIEPKQIKVWFQNRRCREKQRKESSRLQTVNRKLSAMNKLLMEENDRLQKQVSQLVCENGYMRQQLQNVSAATTDVSCESGVTTPQHSLRDANNPAGLLPIAEETLAEFLSKATGTAVDWVPMPGMKPGPDSVGIFAISHSCSGVAARACGLVSLEPTKIADILKDRPSWFRDCRNVEVITMFPAGNGGTVELLYTQIYAPTTLAPARDFWTLRYTTTLDNGSLVVCERSLSGNGPGPNPTAASQFVRAQMLPSGYLIRPCDGGGSIIHIVDHLNLEAWSAPEILRPLYESSKVVAQKMTIAALRYARQLAQETSGEVVYGLGRQPAVPRTFSQRLCRGFNDAINGFGDDGWSMLSSDGAEDVIVAVNSRKNLATTSIPLSPLGGVLCTKASMLLQQNVPPAVLVRFLREHRSE

SEQ ID NO: 8 - DNA, 2529 bp, Arabidopsis thaliana
atggagatggcggtggctaaccaccgtgagagaagcagtgacagtatgaatagacatttagatagtagcggtaagtacgttaggtacacagctgagcaagtcgaggctcttgagcgtgtctacgctgagtgtcctaagcctagctctctccgtcgacaacaattgatccgtgaatgttccattttggccaatattgagcctaagcagatcaaagtctggtttcagaaccgcaggtgtcgagataagcagaggaaagaggcgtcgaggctccagagcgtaaaccggaagctctctgcgatgaataaactgttgatggaggagaatgataggttgcagaagcaggtttctcagcttgtctgcgaaaatggatatatgaaacagcagctaactactgttgttaacgatccaagctgtgaatctgtggtcacaactcctcagcattcgcttagagatgcgaatagtcctgctggattgctctcaatcgcagaggagactttggcagagttcctatccaaggctacaggaactgctgttgattgggttcagatgcctgggatgaagcctggtccggattcggttggcatctttgccatttcgcaaagatgcaatggagtggcagctcgagcctgtggtcttgttagcttagaacctatgaagattgcagagatcctcaaagatcggccatcttggttccgtgactgtaggagccttgaagttttcactatgttcccggctggtaatggtggcacaatcgagcttgtttatatgcagacgtatgcaccaacgactctggctcctgcccgcgatttctggaccctgagatacacaacgagcctcgacaatgggagttttgtggtttgtgagaggtcgctatctggctctggagctgggcctaatgctgcttcagcttctcagtttgtgagagcagaaatgctttctagtgggtatttaataaggccttgtgatggtggtggttctattattcacattgtcgatcaccttaatcttgaggcttggagtgttccggatgtgcttcgacccctttatgagtcatccaaagtcgttgcacaaaaaatgaccatttccgcgttgcggtatatcaggcaattagcccaagagtctaatggtgaagtagtgtatggattaggaaggcagcctgctgttcttagaacctttagccaaagattaagcaggggcttcaatgatgcggttaatgggtttggtgacgacgggtggtctacgatgcattgtgatggagcggaagatattatcgttgctattaactctacaaagcatttgaataatatttctaattctctttcgttccttggaggcgtgctctgtgccaaggcttcaatgcttctccaaaatgttcctcctgcggttttgatccggttccttagagagcatcgatctgagtgggctgatttcaatgttgatgcatattccgctgctacacttaaagctggtagctttgcttatccgggaatgagaccaacaagattcactgggagtcagatcataatgccactaggacatacaattgaacacgaagaaatgctagaagttgttagactggaaggtcattctcttgctcaagaagatgcatttatgtcacgggatgtccatctccttcagatttgtaccgggattgacgagaatgccgttggagcttgttctgaactgatatttgctccgattaatgagatgttcccggatgatgctccacttgttccctctggattccgagtcatacccgttgatgctaaaacgggagatgtacaagatctgttaaccgctaatcaccgtacactagacttaacttctagccttgaagtcggtccatcacctgagaatgcttctggaaactctttttctagctcaagctcgagatgtattctcactatcgcgtttcaattcccttttgaaaacaacttgcaagaaaatgttgctggtatggcttgtcagtatgtgaggagcgtgatctcatcagttcaacgtgttgcaatggcgatctcaccgtctgggataagcccgagtctgggctccaaattgtccccaggatctcctgaagctgttactcttgctcagtggatctctcaaagttacagtcatcacttaggctcggagttgctgacgattgattcgcttggaagcgacgactcggtactaaaacttctatgggatcaccaagatgccatcctgtgttgctcattaaagccacagccagtgttcatgtttgcgaaccaagctggtctagacatgctagagacaacacttgtagccttacaagatataacactcgaaaagatattcgatgaatcgggtcgtaaggctatctgttcggacttcgccaagctaatgcaacagggatttgcttgcttgccttcaggaatctgtgtgtcaacgatgggaagacatgtgagttatgaacaagctgttgcttggaaagtgtttgctgcatctgaagaaaacaacaacaatctgcattgtcttgccttctcctttgtaaactggtcttttgtgtga

SEQ ID NO: 9 - DNA, 2529 bp, Artificial Sequence
  mutated Arabidopsis  REV
atggagatggcggtggctaaccaccgtgagagaagcagtgacagtatgaatagacatttagatagtagcggtaagtacgttaggtacacagctgagcaagtcgaggctcttgagcgtgtctacgctgagtgtcctaagcctagctctctccgtcgacaacaattgatccgtgaatgttccattttggccaatattgagcctaagcagatcaaagtctggtttcagaaccgcaggtgtcgagataagcagaggaaagaggcgtcgaggctccagagcgtaaaccggaagctctctgcgatgaataaactgttgatggaggagaatgataggttgcagaagcaggtttctcagcttgtctgcgaaaatggatatatgaaacagcagctaactactgttgttaacgatccaagctgtgaatctgtggtcacaactcctcagcattcgcttagagatgcgaatagtcctgctggattgctctcaatcgcagaggagactttggcagagttcctatccaaggctacaggaactgctgttgattgggttcagatgcctgggatgaagcctggaccagattcggttggcatctttgccatttcgcaaagatgcaatggagtggcagctcgagcctgtggtcttgttagcttagaacctatgaagattgcagagatcctcaaagatcggccatcttggttccgtgactgtaggagccttgaagttttcactatgttcccggctggtaatggtggcacaatcgagcttgtttatatgcagacgtatgcaccaacgactctggctcctgcccgcgatttctggaccctgagatacacaacgagcctcgacaatgggagttttgtggtttgtgagaggtcgctatctggctctggagctgggcctaatgctgcttcagcttctcagtttgtgagagcagaaatgctttctagtgggtatttaataaggccttgtgatggtggtggttctattattcacattgtcgatcaccttaatcttgaggcttggagtgttccggatgtgcttcgacccctttatgagtcatccaaagtcgttgcacaaaaaatgaccatttccgcgttgcggtatatcaggcaattagcccaagagtctaatggtgaagtagtgtatggattaggaaggcagcctgctgttcttagaacctttagccaaagattaagcaggggcttcaatgatgcggttaatgggtttggtgacgacgggtggtctacgatgcattgtgatggagcggaagatattatcgttgctattaactctacaaagcatttgaataatatttctaattctctttcgttccttggaggcgtgctctgtgccaaggcttcaatgcttctccaaaatgttcctcctgcggttttgatccggttccttagagagcatcgatctgagtgggctgatttcaatgttgatgcatattccgctgctacacttaaagctggtagctttgcttatccgggaatgagaccaacaagattcactgggagtcagatcataatgccactaggacatacaattgaacacgaagaaatgctagaagttgttagactggaaggtcattctcttgctcaagaagatgcatttatgtcacgggatgtccatctccttcagatttgtaccgggattgacgagaatgccgttggagcttgttctgaactgatatttgctccgattaatgagatgttcccggatgatgctccacttgttccctctggattccgagtcatacccgttgatgctaaaacgggagatgtacaagatctgttaaccgctaatcaccgtacactagacttaacttctagccttgaagtcggtccatcacctgagaatgcttctggaaactctttttctagctcaagctcgagatgtattctcactatcgcgtttcaattcccttttgaaaacaacttgcaagaaaatgttgctggtatggcttgtcagtatgtgaggagcgtgatctcatcagttcaacgtgttgcaatggcgatctcaccgtctgggataagcccgagtctgggctccaaattgtccccaggatctcctgaagctgttactcttgctcagtggatctctcaaagttacagtcatcacttaggctcggagttgctgacgattgattcgcttggaagcgacgactcggtactaaaacttctatgggatcaccaagatgccatcctgtgttgctcattaaagccacagccagtgttcatgtttgcgaaccaagctggtctagacatgctagagacaacacttgtagccttacaagatataacactcgaaaagatattcgatgaatcgggtcgtaaggctatctgttcggacttcgccaagctaatgcaacagggatttgcttgcttgccttcaggaatctgtgtgtcaacgatgggaagacatgtgagttatgaacaagctgttgcttggaaagtgtttgctgcatctgaagaaaacaacaacaatctgcattgtcttgccttctcctttgtaaactggtcttttgtgtga

SEQ ID NO: 10 - DNA, 2523 bp, Zea mays
atggttgcagcggtggcgttgcgagggggaagcagcgatagcggaggatttgataaggttcccgggatggactcgggaaaatacgtgcgctacaccccggagcaagttgaggtgctcgagcggctctacatagattgccccaagccaagctcctcacggcggcagcaactgctgcgcgagtgtcctatactctccaacattgagccaaagcagatcaaggtctggttccagaaccggaggtgccgcgataagcagcggaaggagtcttcgcggcttcaggctgtgaacagaaagttgacggcaatgaacaagcttctaatggaagagaatgagcggcttcagaagcaagtctctcagttggttcatgaaaacgcgcacatgcggcagcagctgcagaatacttcattggccaatgacacaagctgtgaatcaaatgtcactacccctccaaaccctataagggacgcaagtaacccttctggactccttgcgattgcagaggagaccttcacagagttcctctcaaaggctactgggacagctattgattgggtccagatgcctgggatgaagcctggtccggattcagttggtatcgtggccatttcgcatggttgccgtggcgttgctgcccgcgcctgtggtttggtgaatctagaaccaacaaaaggcatagagatcttgaaagatcgtccctcttggttccgtgattgccgaagtcttgaagtgtttacaaggtttccagctggaaatgggggaacaattgaacttatttacatgcagatgtatgccccaacaactttagtccctgcacgtgatttttggacactacgatacacgacaacaatggaagatggcagccttgtggtctgtgagagatccttgagtggttctggtggtggtccaaatgcagcctctacacaacaatttgttagggctgagatgcttccaagtgggtatttagttcgcccatgcgaaggtggaggatcaattgtgcatatagtggaccatctagatctcgaagcatggagtgttcctgaagtgcttcgaccactgtatgagtcttctagagttgttgctcagaaaatgactactgtggcactgcgccaccttagacaaattgctcaagaaacaagtggagaagtagtgtacgccttgggaaggcaacctgcagtcctacggacctttagtcaaagactaagcagggggtttaatgatgccattagcggtttcaatgatgatggctggtctgtaatggggggagatggcattgaagacgttgttattgcttgcaactcaaccaagaaaattaggaataccagcaatgctgggattacatttggagccccaggaggcattatttgtgcgaaggcatccatgttactgcagagcgtcccaccggcagtactggtacgatttctgagggagcatagatctgaatgggctgattacaatattgatgcgtatttggcttcatcattgaagaccagtgcgtgctcacttcctgggttgcgacccatgagattttctgaggggcagatgatcatgccacttgctcacacagttgagaacgaggagattcttgaagttgtccgccttgaaggtcagcctcttactcatgatgaagctcttctttcaagggacatccaccttctccagctttgcactggaatagatgagaaatctgtgggttcctccttccagcttgtgtttgcaccaattgatgagcatttcccagatgatgctccattgatttcttctggctttcgtgtcataccacttgatgtgaaaacagatggtgtatcctctggtaggacgctagatttggcatctagtcttgatgtgggctctgctgcaccccaagcctcaggggatgcatctccagatgactgcagtttgagatctgtgctgacaatcgcctttcaattcccgtatgagatgcaccttcaggacagcgttgcagcaatggcccgtcaatatgttcgtagtgtcatttctgctgtgcaaagagtgtcgatggctatatctccctcccaatctggtctaaatgctgggcataggatgctttctggcttccctgaagctgccacacttgctagatgggtttgccagagttatcactaccatctaggtatggaattacttaatcaatcagatggagctggtgaagcattgttgaaaatgctctggcatcatccagatgctgttctgtgctgctcctttaaggagaaacctatgtttacgtttgcaaacaaggcagggctggacatgttagaaacatctcttgttgccctgcaagacctgacgctagacaagatcttcgacgagtcaggaaggaaagcactattctcagacatctcgaaactaatggaacagggctacgcgtacctgccgtcaggcgtgtgcatgtcaggaatgggccgccatgtttctttcgaccaagctgtagcgtggaaggtgctcggcgaggatagcaacatccactgcctggcgttctgcttcgtcaactggtccttcgtgtga

SEQ ID NO: 11 - DNA, 2523 bp, Artificial Sequence
  mutated ZmRLD1 gene
atggttgcagcggtggcgttgcgagggggaagcagcgatagcggaggatttgataaggttcccgggatggactcgggaaaatacgtgcgctacaccccggagcaagttgaggtgctcgagcggctctacatagattgccccaagccaagctcctcacggcggcagcaactgctgcgcgagtgtcctatactctccaacattgagccaaagcagatcaaggtctggttccagaaccggaggtgccgcgataagcagcggaaggagtcttcgcggcttcaggctgtgaacagaaagttgacggcaatgaacaagcttctaatggaagagaatgagcggcttcagaagcaagtctctcagttggttcatgaaaacgcgcacatgcggcagcagctgcagaatacttcattggccaatgacacaagctgtgaatcaaatgtcactacccctccaaaccctataagggacgcaagtaacccttctggactccttgcgattgcagaggagaccttcacagagttcctctcaaaggctactgggacagctattgattgggtccagatgcctgggatgaagcctggaccagattcagttggtatcgtggccatttcgcatggttgccgtggcgttgctgcccgcgcctgtggtttggtgaatctagaaccaacaaaaggcatagagatcttgaaagatcgtccctcttggttccgtgattgccgaagtcttgaagtgtttacaaggtttccagctggaaatgggggaacaattgaacttatttacatgcagatgtatgccccaacaactttagtccctgcacgtgatttttggacactacgatacacgacaacaatggaagatggcagccttgtggtctgtgagagatccttgagtggttctggtggtggtccaaatgcagcctctacacaacaatttgttagggctgagatgcttccaagtgggtatttagttcgcccatgcgaaggtggaggatcaattgtgcatatagtggaccatctagatctcgaagcatggagtgttcctgaagtgcttcgaccactgtatgagtcttctagagttgttgctcagaaaatgactactgtggcactgcgccaccttagacaaattgctcaagaaacaagtggagaagtagtgtacgccttgggaaggcaacctgcagtcctacggacctttagtcaaagactaagcagggggtttaatgatgccattagcggtttcaatgatgatggctggtctgtaatggggggagatggcattgaagacgttgttattgcttgcaactcaaccaagaaaattaggaataccagcaatgctgggattacatttggagccccaggaggcattatttgtgcgaaggcatccatgttactgcagagcgtcccaccggcagtactggtacgatttctgagggagcatagatctgaatgggctgattacaatattgatgcgtatttggcttcatcattgaagaccagtgcgtgctcacttcctgggttgcgacccatgagattttctgaggggcagatgatcatgccacttgctcacacagttgagaacgaggagattcttgaagttgtccgccttgaaggtcagcctcttactcatgatgaagctcttctttcaagggacatccaccttctccagctttgcactggaatagatgagaaatctgtgggttcctccttccagcttgtgtttgcaccaattgatgagcatttcccagatgatgctccattgatttcttctggctttcgtgtcataccacttgatgtgaaaacagatggtgtatcctctggtaggacgctagatttggcatctagtcttgatgtgggctctgctgcaccccaagcctcaggggatgcatctccagatgactgcagtttgagatctgtgctgacaatcgcctttcaattcccgtatgagatgcaccttcaggacagcgttgcagcaatggcccgtcaatatgttcgtagtgtcatttctgctgtgcaaagagtgtcgatggctatatctccctcccaatctggtctaaatgctgggcataggatgctttctggcttccctgaagctgccacacttgctagatgggtttgccagagttatcactaccatctaggtatggaattacttaatcaatcagatggagctggtgaagcattgttgaaaatgctctggcatcatccagatgctgttctgtgctgctcctttaaggagaaacctatgtttacgtttgcaaacaaggcagggctggacatgttagaaacatctcttgttgccctgcaagacctgacgctagacaagatcttcgacgagtcaggaaggaaagcactattctcagacatctcgaaactaatggaacagggctacgcgtacctgccgtcaggcgtgtgcatgtcaggaatgggccgccatgtttctttcgaccaagctgtagcgtggaaggtgctcggcgaggatagcaacatccactgcctggcgttctgcttcgtcaactggtccttcgtgtga

SEQ ID NO: 12 - protein, 840 aa, Zea mays
MVAAVALRGGSSDSGGFDKVPGMDSGKYVRYTPEQVEVLERLYIDCPKPSSSRRQQLLRECPILSNIEPKQIKVWFQNRRCRDKQRKESSRLQAVNRKLTAMNKLLMEENERLQKQVSQLVHENAHMRQQLQNTSLANDTSCESNVTTPPNPIRDASNPSGLLAIAEETFTEFLSKATGTAIDWVQMPGMKPGPDSVGIVAISHGCRGVAARACGLVNLEPTKGIEILKDRPSWFRDCRSLEVFTRFPAGNGGTIELIYMQMYAPTTLVPARDFWTLRYTTTMEDGSLVVCERSLSGSGGGPNAASTQQFVRAEMLPSGYLVRPCEGGGSIVHIVDHLDLEAWSVPEVLRPLYESSRVVAQKMTTVALRHLRQIAQETSGEVVYALGRQPAVLRTFSQRLSRGFNDAISGFNDDGWSVMGGDGIEDVVIACNSTKKIRNTSNAGITFGAPGGIICAKASMLLQSVPPAVLVRFLREHRSEWADYNIDAYLASSLKTSACSLPGLRPMRFSEGQMIMPLAHTVENEEILEVVRLEGQPLTHDEALLSRDIHLLQLCTGIDEKSVGSSFQLVFAPIDEHFPDDAPLISSGFRVIPLDVKTDGVSSGRTLDLASSLDVGSAAPQASGDASPDDCSLRSVLTIAFQFPYEMHLQDSVAAMARQYVRSVISAVQRVSMAISPSQSGLNAGHRMLSGFPEAATLARWVCQSYHYHLGMELLNQSDGAGEALLKMLWHHPDAVLCCSFKEKPMFTFANKAGLDMLETSLVALQDLTLDKIFDESGRKALFSDISKLMEQGYAYLPSGVCMSGMGRHVSFDQAVAWKVLGEDSNIHCLAFCFVNWSFV

SEQ ID NO: 13 - DNA, 2700 bp, Zea mays
gagagctaagagcaacaaggcgacgagatttgtgtggctacgattttgcgttgccgacggaacatgggaacaatggttgcagcggtggcgttgcgagggggaagcagcgatagcggaggatttgataaggttcccgggatggactcgggaaaatacgtgcgctacaccccggagcaagttgaggtgctcgagcggctctacatagattgccccaagccaagctcctcacggcggcagcaactgctgcgcgagtgtcctatactctccaacattgagccaaagcagatcaaggtctggttccagaaccggaggtgccgcgataagcagcggaaggagtcttcgcggcttcaggctgtgaacagaaagttgacggcaatgaacaagcttctaatggaagagaatgagcggcttcagaagcaagtctctcagttggttcatgaaaacgcgcacatgcggcagcagctgcagaatacttcattggccaatgacacaagctgtgaatcaaatgtcactacccctccaaaccctataagggacgcaagtaacccttctggactccttgcgattgcagaggagaccttcacagagttcctctcaaaggctactgggacagctattgattgggtccagatgcctgggatgaagcctggtccggattcagttggtatcgtggccatttcgcatggttgccgtggcgttgctgcccgcgcctgtggtttggtgaatctagaaccaacaaaaggcatagagatcttgaaagatcgtccctcttggttccgtgattgccgaagtcttgaagtgtttacaaggtttccagctggaaatgggggaacaattgaacttatttacatgcagatgtatgccccaacaactttagtccctgcacgtgatttttggacactacgatacacgacaacaatggaagatggcagccttgtggtctgtgagagatccttgagtggttctggtggtggtccaaatgcagcctctacacaacaatttgttagggctgagatgcttccaagtgggtatttagttcgcccatgcgaaggtggaggatcaattgtgcatatagtggaccatctagatctcgaagcatggagtgttcctgaagtgcttcgaccactgtatgagtcttctagagttgttgctcagaaaatgactactgtggcactgcgccaccttagacaaattgctcaagaaacaagtggagaagtagtgtacgccttgggaaggcaacctgcagtcctacggacctttagtcaaagactaagcagggggtttaatgatgccattagcggtttcaatgatgatggctggtctgtaatggggggagatggcattgaagacgttgttattgcttgcaactcaaccaagaaaattaggaataccagcaatgctgggattacatttggagccccaggaggcattatttgtgcgaaggcatccatgttactgcagagcgtcccaccggcagtactggtacgatttctgagggagcatagatctgaatgggctgattacaatattgatgcgtatttggcttcatcattgaagaccagtgcgtgctcacttcctgggttgcgacccatgagattttctgaggggcagatgatcatgccacttgctcacacagttgagaacgaggagattcttgaagttgtccgccttgaaggtcagcctcttactcatgatgaagctcttctttcaagggacatccaccttctccagctttgcactggaatagatgagaaatctgtgggttcctccttccagcttgtgtttgcaccaattgatgagcatttcccagatgatgctccattgatttcttctggctttcgtgtcataccacttgatgtgaaaacagatggtgtatcctctggtaggacgctagatttggcatctagtcttgatgtgggctctgctgcaccccaagcctcaggggatgcatctccagatgactgcagtttgagatctgtgctgacaatcgcctttcaattcccgtatgagatgcaccttcaggacagcgttgcagcaatggcccgtcaatatgttcgtagtgtcatttctgctgtgcaaagagtgtcgatggctatatctccctcccaatctggtctaaatgctgggcataggatgctttctggcttccctgaagctgccacacttgctagatgggtttgccagagttatcactaccatctaggtatggaattacttaatcaatcagatggagctggtgaagcattgttgaaaatgctctggcatcatccagatgctgttctgtgctgctcctttaaggagaaacctatgtttacgtttgcaaacaaggcagggctggacatgttagaaacatctcttgttgccctgcaagacctgacgctagacaagatcttcgacgagtcaggaaggaaagcactattctcagacatctcgaaactaatggaacagggctacgcgtacctgccgtcaggcgtgtgcatgtcaggaatgggccgccatgtttctttcgaccaagctgtagcgtggaaggtgctcggcgaggatagcaacatccactgcctggcgttctgcttcgtcaactggtccttcgtgtgactgacgcccaacccgtcgggtggttatggcgagctgacccttttttgtatggaaaatcatcagctgtgacaaaaggcatatgttgcatgcactagttgaagaaac

SEQ ID NO: 14 - DNA, 2170 bp, Lesquerella fendleri
tcaggaagattaagtctttgcttgttgtctgattttctttaaatacattaagaaatcggttatgaagcttcgttttttgtgttttgggattatgaagctgtctttggatattagttgcggttattagcatgcttctcttttgtgttttggggatgatgaagcagggtctctctatgtaatgcattttgtttgaaaactcagctaatgctaatgcaatttcttttgaaacctttgttatgttttcaaaaatattgaataggttctgttatggatttatttgcaaaagccattgattaaatcaaaccattacataagaacaacattcattattaactaattagagatgcaaaacacaacattacatacaacatcagtgactaattattgagacaaaacaacatcacagacacaaacattcatctcatacatcacttagagagacacaaaaagcaaccaaacacaactattccgccaacaacaattagcttcatacgttttgcttctcctttcaagccttcaatcatcttctcacagccacgaatctgagccttcaataataacatttcttcatcgtgacacttctcacggttatgaatgcaagcctttatgtcctctacttcttctactaaagacacatcggtccacttccaggtgtggaatcctcctcttttgaaatttttctcacaggtatggaataatctacctgggttttttggagttcttgaggttctgatcacaacacggcatccacatcgacaggtcttaggaaaaccacgaaggttatgatcttcaagctcactgtcaaaagataaaaacgagtttgaagaagaagaaggcattatcaatttcagagaattttggagaattttgagagattgagaattgggaaataagaaccctaatccccaatttatgagattgaaaatatatccgttagagaagaaacataatgttgtgcgttttaattagaaaaaatagagatgggctttatcttttgttaagagttttgggcttgggcttgggtttttgataaaaaaattaattaaaccaaaacgacgtcgtttggtttaattgttgttaaaaaaaaattaaaacaccaaaacgacgtcgttttggtgttattaacggccttaaaacggattaaatccataatccgtcagtcaactagggttacggatggtcaacggcgtttttgcataacggaggcacagttcaggcttaacggagtggacggaatggctttttaggaagtttgtaaccggggtcttttgtttatgatgtatttgtccccgtcggctattgttcaggccgtttaggcctttttcctatatactggaaataactattgtccagacgagttacttctccaacatatcaagaagtgttacaaagatgtgttacgaagccataaaactcaaaaccctaagcctaaaccctagaactttctagcacgtttataccttctcctttctttagtttcctttaaaggccttcgtatcataagttttatttttgcttaatactaacactagaaaaaaacaataatcaacataaactaggttaagtcgtggatctaattttattgtgaaaatgtaattgcttctcttaagaaaagattcatagcaaaatattcgcatctttcttgtgaatcatcttttgtttttggggctattaaagaaaaattgaactcatgaaatggtgacaactttattctagaggtaacagaacaaaaatataggaacaacacgtgttgttcataaactacacgtataatactcaagaagatgaatctttataagaatttagttttctcatgaaaacataaaaagttttgtcaattgaaagtgacagttgaagcaaaggaacaaaaggatggttggtgatgatgctgaaatgaaaatgtgtcattcatcaaatactaaatactacattacttgtcactgcctacttctcctctttcctccgccacccattttggacccacgagccttccatttaaaccctctctcgtgctattcaccagaatagaagccaagagagagagagagattgtgctgaggatcattgtcttcttcatcgttattaacgtaagtttttttttgaccacttatatctaaaatctagtacatgcaatagattaatgactgttccttcttttgatattttcagc

SEQ ID NO: 15 - DNA, 119 bp, Arabidopsis thaliana
ttcgattgacagaaaaagactaatttaaatttacgttagagaactcaaatttttggttgttgtttaggtgtctctgttttgttttttaaaattattttgatcaaatgttaaaaaaaaaa

SEQ ID NO: 16 - DNA, 1255 bp, Arabidopsis thaliana
ctttgttttgtagagtgttctatgggttatgatttcgaaaagaaaaaaaattgtgagacacttaataaaattatttcgacaaaaaaagtagcttgtataaaaaaatcagattttaatttatgtaagaacaaattccaatatccaatagttaaaaataattatttgttccgattaatcgagttttgcaaaatatgcacaaaatctatcatgtaccatttctaagactatatatttggttatatattttatgccgtgtgttctgattccaataaattttagcgcatagtaaattttctaaaaagcaaaattttctcaaaagtgtactaatgacaattaattgagtttctacaaaataagaataactattgactcgattttcacaaaactagtatgctaaatatcacattacttttaaaattaaatggaattatctttttcaatattggatacgaataatttttacactaaagttattttaataaaataaccgtttattcaaaatatgtaaagacgacaaaaatatatattaaatggaaaaacgactaacttagtttttgcaaaattaaatggatttgtccttttcaatgtttgaatacaaaaaaaaatctataataagtttattatattaaaataacccgttttttcagaatacgcaaaaacgacaaaaaaatattaattacaaagaaatttagtttatacaaaaatatgaatggctattaatggtgtttactctaaatttaattattatgcatttatgctaaatctttctaaaggtacaaagattcgttttttcaatgtttgaactgcatattaaggtatagatttggaccttaacagagttaatatataaggaagagagccaaggaactccaaaataaaataaagagccttctctctctctctctgagaaaaaacacatatagccaatgaccttctcgtggtcttctgtgccataaaagccattatatacattcaaacacaatctggcgccacatatacacatgtactagtgtatgtatatgtcctaacctctgtattcatatctctctccttgtctgagtggtgcgatgggtatccccataagctgcaaacattgaaccatctgcaacattttgactcgttttcttttgtgtttttccaacatctgtctcttcttcactcgctctctcctaatcaatctccccaacgacctctctttttttttgtttcttcactcagatctctctccctctctctctctctctctccgggaaaa

SEQ ID NO: 17 - DNA, 809 bp, Arabidopsis thaliana
ggttgcatctttgaatacctttttctcatttaggcataacaatataataatttgttttttgttttcattttcttttggtgtcatcttcaaaaatctgtaaacccaaaagtttgtataacttgtttattaagatatttttaattaaatttttttttttgacatttttaaaaaattataaagtgttttatgaatttaaggagtaaataatatttatttagaacactataaattagttttacaagttcttagaaatgtatctgtaaatttcaaaaaggaaaaatatagcatttaattttgaagatttttttctacattatatatatgataaaaatattgtattttgtactttgtagttacaaaaagtcattatatcaacaaatctaaatataaaatatttttctatatattactccaaattaactgtcagaataaaaaagaagaataattattacagaatctgaacattaaaatcgtccctccatatgtggtctctgtctagtccaaaagcaatttacacatcccaagccgaaactatattaaataaacatttttttttctttaactaaaacatttataacatttaacaataaaagttaaaaatcgaacacgtataacgtatttttttacgtatacgtcttgttggcatatatgcttaaaaacttcattacatacatatacaagtatgtctatatatatgatattatgcaaacacaaatctgttgactataattagacttcttcatttactctctctctgacttaaaacatttattttatcttcttcttgttctctctttctctttctctca

SEQ ID NO: 18 - RNA, 20 nt, Arabidopsis thaliana
ugacagaagagagugagcac

SEQ ID NO: 19 - RNA, 21 nt, Arabidopsis thaliana
uugacagaagauagagagcac

SEQ ID NO: 20 - RNA, 20 nt, Arabidopsis thaliana
ucccaaauguagacaaagca

SEQ ID NO: 21 - RNA, 21 nt, Arabidopsis thaliana
uuuggauugaagggagcucua

SEQ ID NO: 22 - RNA, 21 nt, Arabidopsis thaliana
ugccuggcucccuguaugcca

SEQ ID NO: 23 - RNA, 21 nt, Arabidopsis thaliana
uugaaagugacuacaucgggg

SEQ ID NO: 24 - RNA, 21 nt, Arabidopsis thaliana
ucgauaaaccucugcauccag

SEQ ID NO: 25 - RNA, 24 nt, Arabidopsis thaliana
uugaagaggacuuggaacuucgau

SEQ ID NO: 26 - RNA, 21 nt, Arabidopsis thaliana
uggagaagcagggcacgugca

SEQ ID NO: 27 - RNA, 21 nt, Arabidopsis thaliana
ucggaccaggcuucauccccc

SEQ ID NO: 28 - RNA, 21 nt, Arabidopsis thaliana
ucggaccaggcuucauucccc

SEQ ID NO: 29 - RNA, 21 nt, Arabidopsis thaliana
ugaagcugccagcaugaucua

SEQ ID NO: 30 - RNA, 21 nt, Arabidopsis thaliana
ucgcuuggugcaggucgggga

SEQ ID NO: 31 - RNA, 21 nt, Arabidopsis thaliana
cagccaaggaugacuugccga

SEQ ID NO: 32 - RNA, 21 nt, Arabidopsis thaliana
ugauugagccgugucaauauc

SEQ ID NO: 33 - RNA, 21 nt, Arabidopsis thaliana
ugauugagccgcgccaauauc

SEQ ID NO: 34 - DNA, 961 bp, Zea mays
gatccgattgactatctcattcctccaaacccaaacacctcaaatatatctgctatcgggattggcattcctgtatccctacgcccgtgtaccccctgtttagagaacctcccaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacccaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggaagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctaccttaa

SEQ ID NO: 35 - DNA, 1069 bp, Zea mays
gagtcaggtcaacttggccaaacttaaactgtctcggctcgatatattaaggaaccgaataagagaaccaactcgtctcatttgtgagttcgagctctcccgagctaaaaaaacaatatacacatatataaaatagtataaccaattattagttaattctagacctatttaacactaaaaaagagtaacaatactcacactttcacatatcatgtcaatataacaccaaattaacaaatcacttattaattcatccaacacaagtgcgagatttgtttttctgacaaatggttgctcattcaagctaaagagctgactcgaacacagctcgagctggcttgttaacaaatcttgctgagatactagctcagctcgtgacaaaatcaaaatgagctgagctgaattgagtcgagctaaccatgaaccgagcgatctcacgagccacgagtattttgtctagtcctaccaaaaagaccggtccattcttctagtactagtccgaaccccgaaaactttatgatttccatagcatttgtcaaggctgcctcattaatcattttgttgacgatctagagtactctagcgaaaacatgcaagcaaccaaaccgtagagaagtgtagtaggcaaggctggtcgctaatgcgtgcacctggacagtcgtaatcggactgtgcgtgaacgaactaaaaaggcgcaacaaactgttggagtcgctagtcagtacaaaactgaagcggcctttgcccttttgtgactgtcagcacaagcaacaagtccaaactgttgagcacacgatccatccaagtcgacatcctactgctgatcgatcgcgagcttgtcaggttcttcccatccaacgtgcacagctcctatcacggcaaagcaaagcaccagcagcgtacgaggacaaggcctgaatttgttcccaggtgcaacaaacactttttgttctttttagctttgcatccttctcgttccacttacttaatggcacaccatcagcaatgcacaccacggcaacagcattcactgccaagagagtgagcgagcgagcagaggcagcgctgc

SEQ ID NO: 36 - DNA, 2168 bp, Zea mays
ctgcagcctcctctgatatgcatgcctagttactagttgggctaaagtaacccaatttggttatggttacctagctatatagttagttagttagctatcaagtagttccaacacttgcatacaaaaaatagccctcctctagtttaaatgccccaaagctaatttaggttagttttgagctacctagcaattagtacgatccaaacaggctctaagcctattattgatatatatacacataattaagatgggctattggcagtgcaggggatctaggcttgattggcaggtggtgaatgaggtctaggagagtattaagcaatgcaagtgtctctatatatgcatgggggcagctcgcgtacttggtggtagagagggcaccgaccgtcagaagaagaacctaagggtcaaccacagacgacgagagtttatatcgtcattaggttttacaatgtcgtttgcagcttcagcgcgccttggaaaactcggcaagcgataatttcgttgcaacttgataaaaaaatgctatggaagttcatcggatgccttattagcttgagaaaactatttgctatactatactatttctgtttaaattcttagcttacaaatttaattaatgcactaccggaaacgagtgctttgccgagtgtccgaagtactcggcaaagccttaaaaacactcgacaaaggttttaccgagtgtcacactcggcaaagaaggctcggcaaacagtgcatcgtcaaagccttttttgccgagtactttttctcgggcactcggcaaagctttctcgggcactcggcaaacaatacatgtttgccgagtgtttgacactcggcaaacatgtctttgccgattgtggtcatgtgctgagtgtcctgcgctcggtaaatcagctcgttatcgagagccgtactttgccgagtgcggctatcggcaaagccttctttgccgagtgcccgacaaaaggcactcgacaaagagtccgacactcggcaaagcctcggatttcgatagtaatggtgaaattataaatagaatgttataggattcacttaaagaacaaaaaaagtatcttcattatttctttatgtagtaatccgtatacttgctttgcctataaattagagtgtattctcgcttcctgataagttcaaatatttttttgacattgaccaaacatatacaaaacattattaatatttctgataaataataagtatcgttgcattaatagttacttatttgaatatgtaaacattaccaatatttttttgaacttcaccaaacaagttacagccaccaaataagttgctttgtaccaaaaaagttgcagccgccagacttaacactgcacaccatcaaaatcaacgtttcagaccgttagataaccaatatcctttactcattcaaattcaatagacaatactatttggattatctctctccctcacgtcctcggcgcccctctccctcactcgcaccaccgttgtcgccgtcggcaccaccgtcaaccccttccctcgcaccgccaccggtattgtcgacctcttccctcatctctacgggatcatagcgttagcacaaactatatgctagtagatttaatgatctagtgtagtcaaattaaaaaaaattagtgagttactttggtaaccaatagcctccttttctagtatctgtacttccctagagcctagacaaggtgcaagaggaaaccaataaacgactcccaaatttttccaaaaggtttgcacagctcccaagtaaaagctcagttttagtacccgtgcaagttccacgtaggaatttaacagcaaatttttttttgattctttaacaacaaataaataataaaattagtccatctagaccctgtacagttcccacacgtttcaaacggacgaaattaataattacctaaaaatgtgcaggtcgtagcaaagtataattaaattaatacgaaagaataggcacggaaatgccggtaccaaagatataaaaaagggtagaggggaatggaagagtccaggagcaagaaacactcagaagctgcccagagctaccacccttcttatccccacccctcctcctcctaccttttctccttcagacctcaaaatctgtgtgtctcctgccgcggctagctgataggaacaagagc

SEQ ID NO: 37 - DNA, 105 bp, Zea mays
ctgacgcccaacccgtcgggtggttatggcgagctgacccttttttgtatggaaaatcatcagctgtgacaaaaggcatatgttgcatgcactagttgaagaaac

SEQ ID NO: 38 - DNA, 2520 bp, Oryza sativa
atggctgcggcagtggcaatgcgagggagtagcagtgatggaggtggctatgataaggtttccgggatggactccggtaaatatgtgcgctacacgcctgagcaggtggaggcgcttgagcgggtgtacgccgattgccccaagccaacctcctcccgcaggcagcaactgctgcgtgagtgccccatacttgctaacattgagcccaagcagatcaaggtctggttccagaacagaaggtgccgggataagcagcggaaggagtcttcacggcttcaggctgtcaacaggaaattgacggcaatgaacaagctacttatggaagagaatgagcgactccagaagcaggtctcccaattggttcatgagaatgcccacatgcgacagcagctgcagaatactccgctggcaaatgatacaagctgtgaatcaaatgtgactacccctcaaaaccctttaagggatgcaagtaacccctctgggctcctttcaattgcagaggagaccttgacagagttcctctcaaaggctactggtacagctattgattgggtccagatgcctgggatgaagcctggtccggattcggttggtattgtggccatttcacatggttgccgtggtgttgctgcccgtgcctgtggtttggtgaacctagaaccaacaaaagtggtagagatattgaaagatcgtccatcttggttccgtgattgtcgaaacctggaagtctttacaatgattccagcaggaaatggaggaacggttgaacttgtctacacacagttgtatgctccaacaactttagttcctgcacgagatttttggacgttacggtacacaaccacaatggaagatggcagtcttgtggtctgtgagagatctttaagtggttcagggggcggtccaagtgctgcctctgctcagcaatatgtgagagcggaaatgcttccaagtggatacctggttcgcccatgtgaaggtgggggatcaattgtgcacatagtggaccatctggatcttgaggcatggagtgttcctgaggtgcttcggccactctatgaatcttcaagggtagtcgctcagaaaatgactactgcggcactccggcacatcagacaaattgctcaagaaacaagtggggaagtggtgtatgccttggggaggcaaccagcagtgctacggacttttagtcaaaggctgagcagaggctttaacgatgccattagtggtttcaatgatgatgggtggtctataatgggcggagacggtgttgaagatgtagttattgcttgcaactcaactaagaaaattaggagtaacagcaatgctggcatcgcctttggagcccccggaggtattatatgtgctaaggcatcaatgttactgcagagtgttcctccagcagtactggttcgatttctgagggagcatagatctgaatgggccgattacaatattgatgcatatttggcttcaactctgaaaacaagtgcatgttcacttactgggttgcgacccatgagattttctgggagccaaatcatcattccacttgctcacacagttgagaatgaggagattcttgaagttgttcgccttgagggtcaacctcttactcatgatgaagctcttctttcaagggatatccacctgcttcagctctgcactggaatagacgagaagtctgtgggatcctcctttcagcttgtgtttgcaccgattgatgatttcccagatgaaactccattgatttcttctggcttccgtgttataccacttgatatgaaaacagatggtgcatcctctggtaggacattagatttggcatctagtcttgaagtaggttcagcaacagctcaagcctccggagatgcatctgcagatgattgtaacttgcgatctgttctgacgatcgcttttcaattcccttacgagttgcatctccaagacagtgttgcagctatggctcgccaatatgtccgtagcattgtttctgctgtgcaaagagtgtcaatggctatctctccctctcaaactggtctaaatgccggacagaggataatctctggtttccctgaagcagcaacccttgctcgatgggtttgccagagctaccattaccatctaggggtagagttacttagtcaatcagatggagatgcagaacaattgttgaagatgctatggcattaccaagatgctattttgtgctgctcattcaaggaaaaaccggtgtttacatttgccaacaaagcaggactggacatgctagaaacttcccttgtcgccttacaggacctcacattggacaggatctttgatgagcctggaaaagaagcattgttctcaaacattcccaaattgatggagcagggccatgtctacctgccatcaggcgtgtgcatgtcaggaatgggtcggcatgtttctttcgatcaggccgtggcttggaaagtgcttgccgaggatagcaatgttcactgcctggccttctgtttcgtcaactggtcctttgtgtga

SEQ ID NO: 39 - DNA, 2523 bp, Oryza sativa
atggcggcggcggtggcgatgcggagcggcagcggcagcgacggcggcggcggcgggtacgacaaggccgggatggactccggcaagtacgtgcggtacacgccggagcaggtggaggcgctggagagggtgtacgccgagtgccccaagcccagctcctcccgccgccagcagctgctccgcgactgtcccatcctcgccaacatcgagcccaagcagatcaaggtctggttccagaacagaaggtgccgagataagcagcggaaggaggcatcaaggcttcaggccgtgaaccgaaaattgacggcgatgaataagcttctcatggaggagaatgagcgtcttcagaagcaggtctcccagctggtccatgagaacgcgtacatgaagcagcaacttcagaatccgtcattgggcaatgatacaagctgtgaatcaaatgtgaccactcctcagaaccctctgagagatgcaagtaacccgtctggactccttacaattgcggaggagaccctgacagagttcctctccaaggctacagggactgctgttgattgggtgccaatgcctgggatgaagcctggtccggattcgtttggtattgtggccgtttcacatggttgccgtggtgttgctgcccgtgcctgtggtttggtgaatctagaaccaacaaagatcgtggagatcttaaaagaccgcccatcttggttccgtgattgtcgaagtcttgaagtcttcacaatgtttccagctggaaatggtggcacgatcgaacttgtttacatgcagatgtatgctcctactactttggttcctgcacgagatttttggacacttagatacacaactacaatggaggatggcagccttgtggtctgtgagagatcattgagtggttctggaggtggtccaagtacagcctccgcacagcaatttgtaagagctgagatgcttcctagcggctatctagtgcgcccatgcgagggtggtggctccatcgtgcatattgtggaccatctggatcttgaggcttggagtgttccagaagtgcttcggccactctacgagtcatctagggtagttgctcagaaaatgactactgcagcactacggcacatcagacaaattgctcaagagacaagcggggaggttgtatacgctttggggaggcaacctgctgttttgcggacatttagtcagaggttgagtagaggcttcaatgatgctataagtggtttcaatgatgatggttggtctgtcatgggtggggatggcattgaagatgtgatcattgcttgcaatgcaaagaaggttaggaatactagcacttcggccaatgcttttgtaactccaggaggtgttatatgtgctaaggcatccatgctactgcagagtgtcccacctgcagttttggttcgatttttgagggaacatcgttctgaatgggcggattataacttcgatgcatattcagcttcatctctgaagacaagctcatgttcacttcctgggttgcggcctatgagattttctgggagccagatcattatgccacttgctcacacggtggagaatgaggagattttagaagttgtccgtcttgaaggacaagcacttacacatgatgatggtcttatgtctagagatattcacctgcttcagctttgcactggaatagatgagaaatcaatgggatcctgcttccagcttgtctctgcaccaatcgatgagcttttccctgatgatgctccgttaatatcttcaggctttcgtgttataccgctggacatgaaaacagatggtacacctgctggtagaacattagatttggcatctagccttgaagttggttcaactgcacagcccacaggggatgcatctatggatgactgtaatctacgatcagtgctgacaattgcctttcagttcccttatgaaatgcatctccaagacagcgttgcaactatggcccggcaatatgtccgcagtattgtttcctctgttcagagagtatcaatggctatttctccttctcggtctggcttgaatgctgggcagaagataatttcaggcttccctgaagccccaacgctagctcgttggatttgccaaagctaccagttccatttgggggtggagttacttaggcaggcagatgatgctggggaagcactattgaaaatgctatgggattacgaagacgctattttgtgctgttctttcaaggaaaagcctgtgtttacttttgccaacgagatgggactaaacatgctagaaacatctctcgtcgctctccaagatctctcactggacaagatatttgatgaagccggtaggaaggccctatacaacgagatcccgaaattgatggaacagggttacgtgtacctgcctggtggagtgtgcttgtccgggatggggcgccatgtttctttcgagcaagctgtagcatggaaggtgctcggagaagacaacaatgtgcactgcctcgccttctgcttcgtcaactggtccttcgtgtga

SEQ ID NO: 40 - protein, 840 aa, Oryza sativa
MAAAVAMRSGSGSDGGGGGYDKAGMDSGKYVRYTPEQVEALERVYAECPKPSSSRRQQLLRDCPILANIEPKQIKVWFQNRRCRDKQRKEASRLQAVNRKLTAMNKLLMEENERLQKQVSQLVHENAYMKQQLQNPSLGNDTSCESNVTTPQNPLRDASNPSGLLTIAEETLTEFLSKATGTAVDWVPMPGMKPGPDSFGIVAVSHGCRGVAARACGLVNLEPTKIVEILKDRPSWFRDCRSLEVFTMFPAGNGGTIELVYMQMYAPTTLVPARDFWTLRYTTTMDDGSLVVCERSLSGSGGGPSTASAQQFVRAEMLPSGYLVRPCEGGGSIVHIVDHLDLEAWSVPEVLRPLYESSRVVAQKMTTAALRHIRQIAQETSGEVVYALGRQPAVLRTFSQRLSRGFNDAISGFNDDGWSVMGGDGIEDVIIACNAKKVRNTSTSANAFVTPGGVICAKASMLLQSVPPAVLVRFLREHRSEWADYNFDAYSASSLKTSSCSLPGLRPMRFSGSQIIMPLAHTVENEEILEVVRLEGQALTHDDGLMSRDIHLLQLCTGIDEKSMGSCFQLVFAPIDELFPDDAPLISSGFRVIPLDMKTDGTPAGRTLDLASSLEVGSTAQPTGDASMDDCNLRSVLTIAFQFPYEMHLQDSVATMARQYVRSIVSSVQRVSMAISPSRSGLNAGQKIISGFPEAPTLARWICQSYQFHLGVELLRQADDAGEALLKMLWDYEDAILCCSFKEKPVFTFANEMGLNMLETSLVALQDLSLDKIFDEAGRKALYNEIPKLMEQGYVYLPGGVCLSGMGRHVSFEQAVAWKVLGEDNNVHCLAFCFVNWSFV

SEQ ID NO: 41 - DNA, 2523 bp, Oryza sativa
atggcggcggcggtggcgatgcggagcggcagcggcagcgacggcggcggcggcgggtacgacaaggccgggatggactccggcaagtacgtgcggtacacgccggagcaggtggaggcgctggagagggtgtacgccgagtgccccaagcccagctcctcccgccgccagcagctgctccgcgactgccccatcctcgccaacatcgagcccaagcagatcaaggtctggttccagaacagaaggtgccgagataagcagcggaaggaggcatcaaggcttcaggccgtgaaccgaaaattgacggcgatgaataagcttctcatggaggagaatgagcgtcttcagaagcaggtctcccagctggtccatgagaacgcgtacatgaagcagcaacttcagaatccgtcattgggcaatgatacaagctgtgaatcaaatgtgaccactcctcagaaccctctgagagatgcaagtaacccgtctggactccttacaattgcggaggagaccctgacagagttcctctccaaggctacagggactgctgttgattgggtgccaatgcctgggatgaagcctggtccggattcgtttggtattgtggccgtttcacatggttgccgtggtgttgctgcccgtgcctgtggtttggtgaatctagaaccaacaaagatcgtggagatcttaaaagaccgcccatcttggttccgtgattgtcgaagtcttgaagtcttcacaatgtttccagctggaaatggtggcacgatcgaacttgtttacatgcagatgtatgctcctactactttggttcctgcacgagatttttggacacttagatacacaactacaatggatgatggcagccttgtggtctgtgagagatcattgagtggttctggaggtggtccaagtacagcctccgcacagcaatttgtaagagctgagatgcttcctagcggctatctagtgcgcccatgcgagggtggtggctccatcgtgcatattgtggaccatctggatcttgaggcttggagtgttccagaagtgcttcggccactctacgagtcatctagggtagttgctcagaaaatgactactgcagcactacggcacatcagacaaattgctcaagagacaagcggggaggttgtatacgctttggggaggcaacctgctgttttgcggacatttagtcagaggttgagtagaggcttcaatgatgctattagtggtttcaacgatgatggttggtctgtcatgggtggggatggcattgaagatgtgatcattgcttgcaatgcaaagaaggttaggaatactagcacttcggccaatgcttttgtaactccaggaggtgttatatgtgctaaggcatccatgctactgcagagtgtcccacctgcagttttggttcgatttttgagggaacatcgttctgaatgggcggattataacttcgatgcatattcagcttcatctctgaagacaagctcatgttcacttcctgggttgcggcctatgagattttctgggagccagatcattatgccacttgctcacacggtggagaatgaagagattttagaagttgtccgtcttgaaggacaagcacttacacatgatgatggtcttatgtctagagatattcacctgcttcagctttgcactggaatagatgagaaatcaatgggatcctgcttccagcttgtctttgcaccaatcgatgagcttttccctgatgatgctccgttaatatcttcaggctttcgtgttataccgctggacatgaaaacagatggtacacctgctggtagaacattagatttggcatctagccttgaggttggttcaactgcacagcccacaggggatgcatctatggatgactgtaatctacgatcagtgctgacaattgcctttcagttcccttatgaaatgcatctccaagacagcgttgcaactatggcccggcaatatgtccgcagtattgtttcctctgttcagagagtatcaatggctatttctccttctcggtctggcttgaatgctgggcagaagataatttcaggcttccctgaagccccaacgctagctcgttggatttgccaaagctaccagttccatttgggggtggagttacttaggcaggcagatgatgctggggaagcactattgaaaatgctatgggattacgaagacgctattttgtgctgttctttcaaggaaaagcctgtatttacttttgccaacgagatgggactaaacatgctagaaacatctctcgtcgctctccaagatctctcactggacaagatatttgatgaagccggtaggaaggccctatacaacgagatcccgaaattgatggaacagggttacgtgtacctgcctggtggagtgtgcttgtccgggatggggcgccatgtttctttcgagcaagctgtagcatggaaggtgctcggagaagacaacaatgtgcactgcctcgccttctgcttcgtcaactggtccttcgtgtga

SEQ ID NO: 42 - DNA, 2529 bp, Artificial Sequence
  REVstop transgene
atggagatggcggtggctaaccaccgtgagtgaagcagtgacagtatgaattgacatttagatagtagcggtaagtacgttaggtacacagctgagcaagtcgaggctcttgagcgtgtctacgctgagtgtcctaagcctagctctctccgtcgacaacaattgatccgtgaatgttccattttggccaatattgagcctaagcagatcaaagtctggtttcagaaccgcaggtgtcgagataagcagaggaaagaggcgtcgaggctccagagcgtaaaccggaagctctctgcgatgaataaactgttgatggaggagaatgataggttgcagaagcaggtttctcagcttgtctgcgaaaatggatatatgaaacagcagctaactactgttgttaacgatccaagctgtgaatctgtggtcacaactcctcagcattcgcttagagatgcgaatagtcctgctggattgctctcaatcgcagaggagactttggcagagttcctatccaaggctacaggaactgctgttgattgggttcagatgcctgggatgaagcctggtccggattcggttggcatctttgccatttcgcaaagatgcaatggagtggcagctcgagcctgtggtcttgttagcttagaacctatgaagattgcagagatcctcaaagatcggccatcttggttccgtgactgtaggagccttgaagttttcactatgttcccggctggtaatggtggcacaatcgagcttgtttatatgcagacgtatgcaccaacgactctggctcctgcccgcgatttctggaccctgagatacacaacgagcctcgacaatgggagttttgtggtttgtgagaggtcgctatctggctctggagctgggcctaatgctgcttcagcttctcagtttgtgagagcagaaatgctttctagtgggtatttaataaggccttgtgatggtggtggttctattattcacattgtcgatcaccttaatcttgaggcttggagtgttccggatgtgcttcgacccctttatgagtcatccaaagtcgttgcacaaaaaatgaccatttccgcgttgcggtatatcaggcaattagcccaagagtctaatggtgaagtagtgtatggattaggaaggcagcctgctgttcttagaacctttagccaaagattaagcaggggcttcaatgatgcggttaatgggtttggtgacgacgggtggtctacgatgcattgtgatggagcggaagatattatcgttgctattaactctacaaagcatttgaataatatttctaattctctttcgttccttggaggcgtgctctgtgccaaggcttcaatgcttctccaaaatgttcctcctgcggttttgatccggttccttagagagcatcgatctgagtgggctgatttcaatgttgatgcatattccgctgctacacttaaagctggtagctttgcttatccgggaatgagaccaacaagattcactgggagtcagatcataatgccactaggacatacaattgaacacgaagaaatgctagaagttgttagactggaaggtcattctcttgctcaagaagatgcatttatgtcacgggatgtccatctccttcagatttgtaccgggattgacgagaatgccgttggagcttgttctgaactgatatttgctccgattaatgagatgttcccggatgatgctccacttgttccctctggattccgagtcatacccgttgatgctaaaacgggagatgtacaagatctgttaaccgctaatcaccgtacactagacttaacttctagccttgaagtcggtccatcacctgagaatgcttctggaaactctttttctagctcaagctcgagatgtattctcactatcgcgtttcaattcccttttgaaaacaacttgcaagaaaatgttgctggtatggcttgtcagtatgtgaggagcgtgatctcatcagttcaacgtgttgcaatggcgatctcaccgtctgggataagcccgagtctgggctccaaattgtccccaggatctcctgaagctgttactcttgctcagtggatctctcaaagttacagtcatcacttaggctcggagttgctgacgattgattcgcttggaagcgacgactcggtactaaaacttctatgggatcaccaagatgccatcctgtgttgctcattaaagccacagccagtgttcatgtttgcgaaccaagctggtctagacatgctagagacaacacttgtagccttacaagatataacactcgaaaagatattcgatgaatcgggtcgtaaggctatctgttcggacttcgccaagctaatgcaacagggatttgcttgcttgccttcaggaatctgtgtgtcaacgatgggaagacatgtgagttatgaacaagctgttgcttggaaagtgtttgctgcatctgaagaaaacaacaacaatctgcattgtcttgccttctcctttgtaaactggtcttttgtgtga

SEQ ID NO: 43 - DNA, 26 bp, Artificial Sequence
  Primer KpnLec2pr586F
ggtacctgtccatcaacccatgcctc

SEQ ID NO: 44 - DNA, 24 bp, Artificial Sequence
  Primer Lec2-94R
ctgttgtgaagtgcgagcgattgt

SEQ ID NO: 45 - protein, 841 aa, Brassica napus
MAMAVAVGNRHESGENINRHLDSSGKYVRYTGEQVEALERVYSECPKPTSLRRQQLIRECPFLANIEPKQIKVWFQNRRCRDKQRKEASRLQSVNQKLSAMNKLLMEENDRLQKQVSHLVSENGYMQQQLTLTTLGTDASCDSVDPTPPLHPLRDANSPAGLMAIAEETLAEFLSKATGTAVDWVQMPGMKPGPDSVGIFAISQKCYGVAARACGLVSLEPMKIVEILKDRPSWFRDCRSIEVFTMFPAGNGGTIELIYMQTYAPTTLAPARDFWTLRYTTSLEKGSIVVCERSLSGSGAGPNATSAAQFVRAEMLPSGYLIRPCDGGGSIIHIVDHINFEGWSVPDVLRLLYESSKVVAQRMTIAALRYVRQVAHETNGEVVYGLGRQPAVLRTFSQRLSRGFSDAVNGFNDDGWSIMHCNGAEDITVAVNSTKHLNSFSDPLSFLGGVLCAKASMLLQNVCPAVLVRFLREHRSEWADFNVDAYSAATLKAGAFAYSGMRPTTFTGSQIIMPLGNTIEKEEMLEVVRLEGHSLVPEDSFLSRDVHLLQICTGIDEDVVGACSELVFAPVNEMFPDDAPLVPSGFRVIPVDSKTGDAQDLLTANHRTLDLTSSQDVGSTPETGSSPSSRCILTIAFQFPFENNLQENVANMACQYVRSVISSVQRVAVALSPSGLIPIPGSKLSPGSPEAVSLAIWICQSYKQHFGSDLLRTDSLGGDALLRQLWDHQDAILCCSLKPQPVFMFANQAGLDMLETTLVALQDIALEKIFDESGRKALCPDFAKLMQQGFACLPSGMCVSTMGRHVSYEQAVSWKVFSDCEDNNNNRIHCLAFLFANWSFL

SEQ ID NO: 46 - DNA, 2739 bp, Brassica napus
atggcgatggctgtggcggtggggaatcgccatgagagtggtgagaatataaatcggcatttggatagcagcgggaagtatgtaaggtacacgggggagcaagtggaggcactggagcgcgtttactcagaatgtcctaaacccacgtctctccgccgtcaacagctgatcagagaatgcccctttttggccaatattgaacccaagcagatcaaagtctggtttcaaaaccgaaggtgtcgagataaacagaggaaagaggcgtcgaggctgcagagcgtaaaccagaagctttcagctatgaataagctgctgatggaggagaatgataggcttcaaaaacaggtttctcatcttgtcagtgaaaacggatacatgcagcagcagctcaccttgaccacactcgggactgatgctagctgcgactcggtggatccaactcctcctctgcatccccttagagatgcaaatagtcctgcaggattgatggcgattgcagaggagaccttggcagagttcctttcaaaggctacaggaactgctgttgattgggtccagatgcctggtatgaagcctggtccggattcggttggcatttttgccatttcacaaaaatgctatggcgtggcagctcgagcctgtggtcttgtcagtttagaacctatgaagattgttgagatactcaaagatcgaccatcttggttccgcgactgccggagcattgaagttttcacaatgttccctgctggtaatggtggaacgattgaactcatatatatgcagacatatgcaccgacgactctggctcctgcccgtgacttctggaccctgagatacactacaagccttgagaagggcagtattgtggtttgtgagaggtctctctctggttcaggagctggtcctaatgctacttccgctgctcagtttgtgagggcagagatgcttcctagtggctatttaataaggccttgtgatggtggtggttccataattcacatcgtcgatcacattaattttgagggttggagtgtaccagacgtgctgcggctcctttatgaatcttccaaagtagttgcacagagaatgacaattgctgcgttgagatatgtgaggcaagtagcgcatgagacaaatggagaagtggtatacggattaggtagacagcctgcggttttgagaacttttagccaaagactaagcagaggtttcagtgatgcggttaatgggtttaatgacgatgggtggtccataatgcattgtaatggcgcagaagacataactgttgctgttaactctacgaagcacttgaatagtttttctgatccactctcttttctaggaggtgtgctctgcgccaaggcttcaatgcttcttcaaaatgtttgtcctgcggttctcgttcgatttctaagggagcatcggtctgagtgggctgatttcaatgttgatgcttattctgctgccacgctgaaagctggtgcttttgcttattccggaatgaggcctacaacgttcaccggtagccaaatcataatgccattagggaatacaatcgaaaaggaagagatgctagaagttgttagattagaagggcattctctagtcccagaagactcatttttgtctagggatgtacatcttctacagatttgtactgggattgatgaggatgttgttggagcttgttcagagcttgtatttgctccagtcaatgagatgtttcctgatgatgctccccttgttccttctggtttccgagtcatacctgttgattctaaaacgggagatgctcaggacttgcttactgctaaccaccggacattagacttaacttcaagccaagatgtgggttctacaccagagacgggttctagtccgagctcacggtgcatactcacgattgcttttcagttcccctttgagaataacttgcaagagaatgttgccaacatggcttgtcagtatgtgcggagtgtgatttcctcggttcaacgggttgcagtggcgctctcaccgtctgggttaatcccgattcctggttccaagttgtctccagggtcccctgaagctgtttctcttgcaatatggatctgccaaagttacaaacaacactttggctctgatttgctgaggacagactcacttggaggtgatgcgttattaagacaactatgggatcaccaagatgctatattgtgttgctctttgaagccacagccagtgttcatgtttgcgaaccaggcaggtctagacatgttagagacaacgctcgttgcgttacaagacatcgcattggagaagatctttgacgagtcaggtcgtaaagccctctgcccagattttgccaagctaatgcaacagggatttgcttgcttaccatctggaatgtgcgtgtcgacaatggggagacatgtttcgtacgagcaagctgtttcatggaaagtgttctctgactgtgaagacaataacaacaatagaattcattgtcttgctttcttgtttgcgaactggtcgttcctctgatctctgatcctcttgttgtctttaagggggtatcctttactcattttttattgatttaggtgctttattttgtaatttatttgttcttatttagtagctttcggaaataaaaaaaaaaaaaaaaaaaaaaaaaaaaaaaaaaaaaaaaaaaaaaaaaaaaaaaaaaaaaaaaaaaaaaaaaaaaaaaaaaaaaaacaaaagaaaaaaaaaaaa

SEQ ID NO: 47 - protein, 841 aa, Glycine max
MAMAVAQHRESSSSGSIDKHLDSGKYVRYTAEQVEALERVYAECPKPSSLRRQQLIRECPILSNIEPKQIKVWFQNRRCREKQRKEASRLQTVNRKLTAMNKLLMEENDRLQKQVSQLVCENGFMRQQLHTPSAATTDASCDSVVTTPQHTMRDANNPAGLLSIAEETLTEFLSKATGTAVDWVQMPGMKPGPDSVGIFAISQSCSGVAARACGLVSLEPTKIAEILKDRPSWFRDCRSLEVFTMFPAGNGGTIELVYTQTYAPTTLAPARDFWTLRYTTSLENGSLVVCERSLSGSGTGPNPAAAAQFVRAETLPSGYLIRPCEGGGSIIHIVDHLNLEAWSVPEVLRPLYESSKVVAQKMTIAALRYIRQIAQETSGEVVYGLGRQPAVLRTFSQRLSRGFNDAVNGFNDDGWTVLNCDGAEDVFIAVNSTKNLSGTSNPASSLTFLGGILCAKASMLLQNVPPAVLVRFLREHRSEWADFSVDAYSAASLKAGTYAYPGMRPTRFTGSQIIMPLGHTIEHEEMLEVIRLEGHSLAQEDAFVSRDIHLLQICSGIDENAVGACSELVFAPIDEMFPDDAPLIPSGFRIIPLDSKPGDKKEVATNRTLDLTSGFEVGPATTAGTDASSSQNTRSVLTIAFQFPFDSSLQDNVAVMARQYVRSVISSVQRVAMAISPSGISPSVGAKLSPGSPEAVTLAHWICQSYSYYIGSDLLRSDSLVGDMMLKQLWHHQDAILCCSLKPLPVFIFANQAGLDMLETTLVALQDITLDKIFDEAGRKALCTDFAKLMEQGFAYLPAGICMSTMGRHVSYDQAIAWKVLTGEDNTVHCLAFSFINWSFV

SEQ ID NO: 48 - DNA, 2526 bp, Glycine max
atggctatggctgtggctcaacacagagaaagtagcagcagtggaagcattgacaaacacttagattctggcaagtatgtgaggtacactgccgagcaagttgaggctcttgaaagggtctatgctgagtgccctaagcctagttctctgagaaggcaacaattgatcagagagtgccccattctctccaacatcgagcctaagcaaatcaaggtttggttccagaaccgcaggtgtagggagaagcagagaaaagaggcatctaggcttcagactgtgaaccgcaaactcactgcaatgaacaagttgttgatggaggagaatgatcggttgcagaagcaggtgtcacagcttgtgtgtgagaatgggtttatgaggcagcaattgcatactccatcagcagcaactactgatgctagttgtgattcggtggttaccactcctcagcataccatgagagatgctaataaccctgctggactcctatcaattgcagaggaaactttgacagagttcctttcaaaggctacaggaactgctgtagattgggtccagatgcctgggatgaagcctggtccggattcggttgggatctttgccatttcacaaagttgcagtggagtggcagctcgagcctgtggtcttgttagtttagaacctacgaagattgcagagatccttaaagatcgtccatcttggttccgtgactgtcggagcctagaagtttttactatgtttcctgctggaaatggaggaaccattgaacttgtttacacgcagacatatgctccaacaacactggctcctgcccgggatttctggacgctgagatataccaccagcttggaaaatggtagtcttgtggtttgtgagaggtccctgtctggttctggaacaggccctaatccagctgctgctgctcagtttgtaagagctgaaacacttcctagtggctacttaatccgaccatgtgagggtggagggtcaatcattcacatagtggaccacctaaatctggaggcatggagtgtgccagaagtgttgcggccactttatgaatcatccaaggtggttgctcagaaaatgacaattgcagcgcttcgctatatcaggcaaatagctcaggaaacaagtggtgaggtggtttacggattgggtaggcagcctgctgttctgcggactttcagccagagattgagcagaggcttcaatgacgctgtaaatggattcaatgatgatgggtggactgtattgaactgtgatggtgccgaggatgtatttattgcagttaattcaaccaagaatttgagtggcacttctaacccagcaagttcccttacattccttggaggaattctctgtgcaaaagcttctatgttgcttcaaaatgtccctcctgcagttttggttcgttttctgagggagcaccgctcagagtgggctgatttcagtgttgatgcttattctgctgcatcactgaaagccggcacctatgcctatccagggatgaggcctacaagatttactggcagtcaaataattatgcctcttggtcatacaattgaacatgaagagatgctggaagttattaggctggaaggtcactctcttgctcaagaagatgcttttgtttctagggacattcatctcttacagatatgtagtgggattgatgagaatgctgtgggggcttgttctgagcttgtatttgctccaattgatgaaatgttccccgatgatgctccattgattccttctggtttccgcattatcccattagattcaaaaccaggtgacaaaaaggaagttgctacaaatcggaccctggatttgacatctggttttgaagtgggtcctgcaacaactgctggcacagatgcatcatccagtcaaaacactcgatcagtgttgactattgccttccagttccctttcgacagcagtctgcaagacaatgtcgcagtcatggcacgtcagtatgtccgcagtgtgatttcttccgtgcagagggttgccatggctatatcaccatctggtataagcccatctgttggagctaaactttctcctggttctccagaggctgttacactagctcactggatctgccaaagttatagttactatatagggtctgacttactgagatccgattctcttgtgggtgacatgatgctgaaacaactgtggcatcatcaggatgcaattctatgctgttcactgaagccattgccagtgttcatatttgcaaatcaagctggccttgacatgttggaaacaactctagtagctttacaagacatcacattggataaaatatttgatgaggctggacgcaaggcattgtgtacagactttgccaagttaatggagcagggatttgcttatctgccagctggaatctgcatgtcgacgatgggacgccatgtgtcatatgaccaagccatcgcatggaaagtgcttactggagaagacaacactgttcactgcctggctttctctttcataaattggtcatttgtatga

SEQ ID NO: 49 - protein, 842 aa, Glycine max
MAMVVAQHRESSSSGSIDKHLDSGKYVRYTAEQVEALERVYAECPKPSSLRRQQLIRECPILSNIEPKQIKVWFQNRRCREKQRKEASRLQTVNRKLTAMNKLLMEENDRLQKQVSQLVCENGFMRQQLHTPSATTTDASCDSVVTTPQHTLRDASNPAGLLSIAEETLTEFLSKATGTAVDWVQMPGMKPGPDSVGIFAISQSCSGVAARACGLVSLEPTKIAEILKDRPSWFRDCRSLEVFTMFPAGNGGTIELVYTQTYAPTTLAPARDFWTLRYTTSLENGSLVVCERSLSGSGTGPNPAAAAQFVRAETLPSGYLIRPCEGGGSIIHIVDHLNLEAWSVPEVLRPLYESSKVVAQKMTIAALRYIRQIAQETSGEVVYGLGRQPAVLRTFSQRLSRGFNDAVNGFNDDGWTVLNCDGAEDVIIAVNSTKNLSGTSNPASSLTFLGGILCAKASMLLQNVPPAVLVRFLREHRSEWADFNVDAYSAASLKAGTYAYPGMRPTRFTGSQIIMPLGHTIEHEEMLEVIRLEGHSLAQEDAFVSRDIHLLQICSGIDENAVGACSELVFAPIDEMFPDDAPLVPSGFRIIPLDSKPGDKKDAVATNRTLDLTSGFEVGPATTAGADASSSQNTRSVLTIAFQFPFDSSLQDNVAVMARQYVRSVISSVQRVAMAISPSGINPSIGAKLSPGSPEAVTLAHWICQSYSYYLGSDLLRSDSLVGDMMLKQLWHHQDAILCCSLKSLPVFIFANQAGLDMLETTLVALQDITLDKIFDEAGRKALCTDFAKLMEQGFAYLPAGICMSTMGRHVSYDQAIAWKVLTGEDNTVHCLAFSFINWSFV

SEQ ID NO: 50 - DNA, 2529 bp, Glycine max
atggctatggttgtggctcaacacagagaaagtagcagcagtggaagcattgacaagcacttagattctggtaagtatgtgaggtatactgctgagcaagttgaggctcttgaaagggtctatgccgagtgccctaagcctagttctctgcggaggcaacaattgatcagagagtgccccattctctccaacatcgagcctaagcaaatcaaggtttggttccagaaccgtaggtgtagggagaagcaaagaaaagaggcatctaggcttcagaccgtgaaccgcaaactcactgcaatgaacaagttgttgatggaggagaatgatcggttgcagaagcaggtgtcacagcttgtttgtgagaatggttttatgaggcagcaattgcatactccatcggcaacaactactgatgctagttgtgattcggtggttaccactcctcagcataccctgagagatgctagtaaccctgctggactcctatcaattgcagaggaaactttgacagagttcctttcaaaggctacaggaactgctgtagattgggtccagatgcctgggatgaagcctggtccggattcggttgggatctttgccatttcacaaagttgcagtggagtggcagctcgagcctgtggtcttgttagtttagaacctacaaagattgcagagatccttaaagatcgtccatcttggttccgtgactgtcggagcctagaagtttttactatgtttcctgctggaaatggaggaaccattgaacttgtttacacacagacatatgctccaacaacactggctcctgctcgggatttctggactctgagatataccacaagtttggaaaatggcagtcttgtggtttgtgagaggtccctgtctggttctggaactggccctaatccagctgctgctgctcagtttgtaagggctgaaacactccctagtggctacttgatccgaccatgtgaaggtggagggtcaatcattcacatagtagaccacctaaatctcgaggcatggagtgtgccagaagtgttgcggccactttatgaatcatccaaggtggttgctcagaaaatgacaattgcagcgcttcgctatatcaggcaaatagctcaggaaacaagtggtgaggttgtttatggattgggtaggcagcctgctgttctgcggactttcagccagagattgagcagaggcttcaatgacgctgtaaatggattcaatgatgatgggtggactgtattgaactgtgatggtgctgaggatgtaattattgctgttaattcaaccaagaatttgagtggcacttctaacccagcaagttcccttacattccttggaggaattctctgtgcaaaagcttctatgttgcttcaaaatgtccctcctgcagttttggttcgttttctgagggagcaccgctcagagtgggctgatttcaatgttgatgcttattctgctgcatcactgaaagctggcacctatgcctatccagggatgaggcctacaagattcactggcagtcaaataattatgcctcttggtcatacaattgaacatgaagagatgcttgaagttattaggttggaaggtcactctcttgctcaagaagacgcttttgtttctagggacattcatctcttacagatatgtagtgggattgatgagaatgctgtgggggcttgttcagagctcgtatttgctccaattgatgaaatgttccctgatgatgctccattggttccttctggtttccgcattatcccattagattcaaaaccaggtgacaaaaaggatgctgttgctacaaatcggaccctggatttgacatctggttttgaagttggtcctgcaacaactgctggtgcagatgcatcatcaagtcaaaacactcgatcggtgttgactattgccttccagttcccttttgacagcagtctgcaagacaacgtcgcggtcatggcacgccaatatgtccgcagtgtgatttcctccgtgcagagggttgccatggctatatctccatctggtataaacccatcaattggagctaaactttctcctggttctccagaggctgttacactagctcactggatctgccaaagttatagttactatttagggtccgacttactgagatcagattctcttgttggtgacatgatgctgaaacaactgtggcatcaccaggatgccattctatgctgttcactgaagtcattgccagtgttcatatttgcaaatcaagctggccttgacatgttggaaacaaccctagttgccttacaagatatcacattggataaaatatttgatgaggctggacgcaaggcattgtgtacagactttgccaagttaatggagcagggttttgcttatctgccagctggaatctgcatgtcgacgatggggcgccatgtgtcatatgaccaagccattgcatggaaagttcttactggggaagacaacactgttcactgcttggctttctctttcataaattggtcatttgtatga

(19) United States
(12) Patent Application Publication
(10) Pub. No.: US 2011/0271405 A1
(43) Pub. Date: Nov. 3, 2011

(54) COMPOSITIONS AND METHODS FOR INCREASING SEED SIZE AND/OR YIELD BY EXPRESSING A MODIFIED TRANSGENE ENCODING A GROWTH AND/OR DEVELOPMENT RELATED PROTEIN

(75) Inventors: Thu NGUYEN, Brier, WA (US); Jay Derocher, Bothell, WA (US)

(73) Assignee: TARGETED GROWTH, INC., SEATTLE, WA (US)

(21) Appl. No.: 12/868,665

(22) Filed: Aug. 25, 2010

Related U.S. Application Data
(60) Provisional application No. 61/236,830, filed on Aug. 25, 2009.
(60) Provisional application No. 61/236,824, filed on Aug. 25, 2009.

(51) Int. Cl. A01H 1/00; C12N 5/10; A01H 5/00

(52) U.S. Cl. 800/290; 800/298; 800/306; 800/320.1; 800/320.2; 800/320.3; 800/312; 800/320; 435/419; 435/412; 435/415

(57) ABSTRACT

The present disclosure provides methods and materials for altering the phenotype of a plant by expressing a modified transgene encoding a growth and/or development related protein. Transformed plants that express the modified transgene present a phenotype that includes increased seed size and/or number as compared with wild-type plants.

### CROSS REFERENCE TO RELATED APPLICATIONS

The present application claims the benefit of U.S. Provisional Application Nos. 61/236,830 filed on Aug. 25, 2009, and 61/236,824 filed on Aug. 25, 2009, each of which is herein incorporated by reference in its entirety for all purposes.

### TECHNICAL FIELD

The application relates generally to plant molecular biology. In particular, it relates to compositions and methods to regulate expression of targeted sequences.

### BACKGROUND

Global factors such as climate change, population growth and the adoption of crops for biofuels create the necessity to develop novel approaches to significantly increase crop yields. Significant yield enhancements achieved in the past 60 years were achieved by improved agronomic practice, large scale use of nitrogen fertilizer and pesticides, and improved genetics and hybrid vigor. Continuing advances in crop productivity have come to rely on germplasm improvement primarily through classical breeding. This approach currently gives yield increases in the range of 1.0-1.5% per year in the major food crops (Calderini and Slafer 1998, Field Crops Research, 57(3): 335-347; Egli 2008, Agronomy Journal, 100: S79-S88). Due to rapid population growth, income growth in developing countries, limited availability of land and climate change, achieving sustainable food security will require technological advances in agronomic practices, breeding and agricultural biotechnology (Dyson 1999, PNAS, 96(11): 5929-5936; Pinstrup-Andersen et al 1999, World Food Prospects Critical issues for the Early Twenty-first Century, in 2020 Vision Food Policy Report). Identification and manipulation of specific genes that play a significant role in determining intrinsic yield could provide a path to obtain substantial yield increases in a relatively short time.

REVOLUTA (REV) is a homeodomain leucine zipper transcription factor belonging to subfamily III (HD-ZIP III) that has multiple functions in plant development. It controls meristem and organ growth, establishes cell fate and polarity, and controls vascular development (Talbert et al 1995, Development, 121(9): 2723-2735; Otsuga et al 2001, Plant Journal, 25(2): 223-236; Zhong and Ye 1999, Plant Cell, 11(11): 2139-2152).

REV, along with other HD-Zip III family members, are among the transcription factors subject to micro RNA (miRNA) regulation. miRNAs originate from distinct loci within a plant's genome and are short non-coding RNAs (20-24 nucleotides (nt) in length) whose function is to repress the expression of defined target genes (Rhoades et al., Cell 110:513-520, 2002; Bonnet et al., Proc. Natl. Acad. Sci. USA, 101:11511-11516, 2004; Reinhart et al., Genes Dev. 16:1616-1626, 2002). miRNAs are generated from longer precursor molecules by a Dicer-like (DCL) ribonuclease and get incorporated into ribonucleoprotein silencing complexes that effect repression of target mRNAs via base pairing of the small RNA and its target mRNA (Chen, Science 303:2022-2025, 2004; Bao et al., Dev. Cell. 7:653-662, 2004). REV and the other four members of the HD-Zip III family have miRNA binding sites in their START (sterol lipid binding) domains that are complementary to the miRNAs designated 165 and 166. A number of studies done in recent years have supported the idea that class III HD-Zip mRNAs are repressed in a spatially-specific manner by miRNA 165/166 and that this repression is essential in, for example, normal adaxial/abaxial fate specification, development of axillary shoot apical meristems (SAMs), and vascular development (McConnell and Barton, Development 125:2935-2942, 1998; McConnell et al., Nature 411:709-713, 2001; Emery et al., Curr. Biol. 13:1768-1774, 2003; Juarez et al., Nature 428:84-88, 2004, Zhong and Ye, Plant Cell Physiol. 45:369-385, 2004; Kim et al., Plant J. 42:84-94, 2005; Ochando et al., Plant Physiol. 141:607-619, 2006; Zhou et al., Plant Cell Physiol. 48:391-404, 2007; Ochando et al., Int. J. Dev. Biol. 52:953-961, 2008).

Canola over expressing an Arabidopsis thaliana Revoluta (At REV) transgene in an embryo-specific manner gave a 15% seed yield increase in replicated yield trials across multiple years (WO20077079353). There are two straightforward interpretations of these results: i) the REV transgene functions at the protein level to cause the yield increase, or ii) the REV gene functions at the transcriptional level to cause the yield increase.

To distinguish between the opposing protein and transcript models, the present invention generated plants carrying a modified REV transgene that contained mutations in the miRNA binding site such that endogenous miRNAs could no longer bind to the REV transgene or a modified REV transgene that did not code for a full-length REV protein. Introducing early stop codons into this translational REV mutant transgene would prevent expression of full length REV protein from the transgene. An mRNA surveillance system called nonsense-mediated decay (NMD) exists in all eukaryotes, including plants, to degrade native mRNAs as well as heterologous mRNAs with premature termination codons (Gutierrez et al., Trends Plant Sci. 4:429-438, 1999; Maquat, Nat. Rev. Mol. Cell. Biol. 5:89-99, 2004; Baker and Parker, Curr. Opin. Cell Biol. 16:293-299, 2004). Degradation of mRNAs containing nonsense mutations ensures that potentially detrimental small polypeptides do not accumulate in the organism.

Therefore, the present invention provides compositions and methods to increase seed number and/or size which leads to increased yield in plants by expressing modified nucleic acids/genes encoding at least one growth and/or development related protein.

### BRIEF SUMMARY

The present invention provides a modified plant growth and/or development gene. In some embodiments, the modified gene has a mutated miRNA binding site, or one or more early stop codons.

The present invention provides plants comprising one or more modified plant growth and/or development nucleic acids/genes of the present invention, as well as compositions and methods for producing such plants. In some embodiments, the modified nucleic acids/genes have a mutated miRNA binding site, and/or one or more early stop codons. In some further embodiments, the modified plant growth and/or development nucleic acids/genes are operatively associated with a promoter, such as an embryo-specific promoter, an endosperm-specific promoter, or an ear-specific promoter, and optionally with a polyA sequence, wherein the plants of the present invention have an increase in seed number and/or seed size as compared with a wild-type plant which does not comprise the modified nucleic acids/genes. In some embodiments, the embryo specific promoter is an early phase-specific embryo promoter. In some embodiments, the resultant increase in seed number and/or seed size leads to increased yield. In some embodiments, the plants of the present invention are transgenic plants. In some other embodiments, the plants of the present invention are non-transgenic plants, such as for example, a plant with natural mutations, or a mutant plant generated from non-transgenic mutagenesis.

In some embodiments, the plant growth and/or development nucleic acid/gene is a HD-Zip transcription factor, a NAC-containing transcription factor, a BHLH transcription factor, a MYB transcription factor, an APETALA2-like transcription factor, a SBP-like transcription factor, a SCL transcription factor, an ARF transcription factor, an F-box protein. The HD-Zip transcription factor can be the REVOLUTA (REV) gene, PHABULOSA (PHB), PHAVOLUTA (PHV), ATHB8, or ATHB15; the NAC-containing transcription factor can be NAC1, CUC1, or CUC2; the BHLH transcription factor can be TCP2, TCP3, TCP4, TCP10, or TCP24; the MYB transcription factor can be MYB33, MYB65, or GAMYB; the APETALA2-like transcription factor can be AP2, TOE1, TOE2, TOE3, or GL15; the SBP-like transcription factor can be SPL3, SPL4, or SPL5, the SCL transcription factor can be SCL6-II, or SCL6-III, the ARF transcription factor can be ARF6, ARF10, ARF16, ARF17, or ARF18; and the F-box protein can be TIR1. In certain embodiments described herein the REV gene can encode a polypeptide comprising the full or partial REV from Arabidopsis thaliana (e.g., SEQ ID NO: 1, encoded by SEQ ID NO: 8), Brassica napus, camelina, soybean, wheat, rice (e.g., OsREV1, SEQ ID NO: 2, encoded by SEQ ID NO: 38; OsREV2, SEQ ID NO: 3, encoded by SEQ ID NO: 39; or TGI OsREV2, SEQ ID NO: 40, encoded by SEQ ID NO: 41), corn (e.g., ZmRLD1, SEQ ID NO: 12, encoded by SEQ ID NO: 10, or ZmRLD2, SEQ ID NO: 4, encoded by SEQ ID NO: 5), tomato (e.g., SEQ ID NO: 7) or sorghum. In some embodiments, the REV gene can encode a variant derived from the REV in Arabidopsis thaliana, Brassica napus, camelina, soybean, wheat, rice, corn, or sorghum, with at least 60%, at least 65%, at least 70%, at least 75%, at least 80%, at least 85%, at least 90%, at least 95%, at least 96%, at least 97%, at least 98%, at least 99% or more sequence identity to their counterpart wild type sequences.

In some embodiments, the promoter used in the present invention is an embryo promoter, an endosperm-specific promoter, or an ear-specific promoter which is homologous or heterologous to the plant. In some embodiments, the embryo specific promoter is an early phase-specific embryo promoter. The early phase-specific embryo promoter can be the promoter associated with an amino acid permease gene (AAP1), an oleate 12-hydroxylase: desaturase gene, a 2S2 albumin gene (2S2), a fatty acid elongase gene (FAE1), a leafy cotyledon 2 gene (LEC2), a leafy cotyledon 1 (LEC1) gene, an aspartic protease 1 gene (ASP1), or an oleosin gene, and wherein the endosperm-specific promoter can be the promoter associated with a legumin 1A (LEG1A) gene, and wherein the ear-specific promoter can be the promoter associated with an AGAMOUS gene or a CLAVATA 1 gene (CLV1). For example, the AAP1 promoter is the AAP1 promoter from Arabidopsis thaliana (SEQ ID NO.: 17), or functional part thereof, the oleate 12-hydroxylase:desaturase promoter is the oleate 12-hydroxylase:desaturase gene promoter from Lesquerella fendleri (LFAH12, SEQ ID NO: 14), or functional part thereof, the 2S2 gene promoter is from Arabidopsis thaliana, the fatty acid elongase gene promoter is from Arabidopsis thaliana, the leafy cotyledon gene promoter is from Arabidopsis thaliana (SEQ ID NO: 16), or functional part thereof, the oleosin gene promoter is from Zea mays (SEQ ID NO: 34), or functional part thereof, the leafy cotyledon 1 (LEC1) gene promoter is from Zea mays (ZmLEC1), or functional part thereof, the aspartic protease 1 (ASP1) gene promoter is from Oryza sativa or Zea mays (OsAsp 1; ZmAsp 1), or functional part thereof, the legumin 1A (LEG1A) gene promoter is from Zea mays (ZmLEG1A, SEQ ID NO: 35), or functional part thereof, the AGAMOUS gene promoter is from Zea mays (ZmZAG1, SEQ ID NO: 36), or functional part thereof, or the CLAVATA 1 gene promoter is from Zea mays (ZmCLV1), or functional part thereof.

In some embodiments, the REV gene is from Arabidopsis thaliana, Zea mays, Brassica napus, camelina, soybean, rice, sorghum, or wheat. For example, in one embodiment, the plants comprise a mutant Arabidopsis thaliana REV gene, in which the Revoluta coding sequence (SEQ ID NO. 8) is mutated such that a Thymidine at nucleotide 567 is changed to an Adenine and a Guanidine at nucleotide 570 is changed to an Adenine. In another embodiment, the plants comprise a Zea mays mutant REV gene, in which the Revoluta coding sequence (Zm RLD1, SEQ ID NO. 10) is mutated such that a Thymidine at nucleotide 579 is changed to an Adenine and a Guanidine nucleotide 582 is changed to an Adenine. Still in another embodiment, the plants comprise a mutant Arabidopsis thaliana REV gene, in which the Revoluta coding sequence is mutated such that a stop codon is encoded at amino acid residue positions 11 and 18.

The present invention also provides transformed cells, tissue cultures and/or plant parts comprising the modified plant growth and/or development nucleic acids/genes of the present invention. The transformed cell, tissue culture or plant part can be derived from regenerable cells from embryos, protoplasts, meristematic cells, callus, pollen, leaves, anthers, stems, petioles, roots, root tips, fruits, seeds, flowers, cotyledons, or hypocotyls. In some embodiments, the modified plant growth and/or development nucleic acid/gene has a mutated miRNA binding site, or one or more early stop codons. In some embodiments, the modified plant growth and/or development nucleic acid/gene is operatively associated with a promoter, such as for example, an embryo-specific promoter, an endosperm-specific promoter, or an ear-specific promoter, and optionally a polyA sequence, wherein the transformed cell, tissue culture or plant part can give rise to a transgenic plant demonstrating an increase in seed number and/or seed size as compared with a wild-type plant or a plant which does not comprise the mutated plant growth and/or development nucleic acid/gene. In some embodiments, the embryo specific promoter is an early phase-specific embryo promoter. In some embodiments, the embryo-specific promoter, the endosperm-specific promoter, or the ear-specific promoter is a promoter described herein. For example, the promoter can be an AAP1 promoter from Arabidopsis thaliana (AtAAP1), an oleate 12-hydroxylase:desaturase gene promoter from Lesquerella fendleri (LFAH12), a 2S2 gene promoter from Arabidopsis thaliana (At2S2), a fatty acid elongase gene promoter from Arabidopsis thaliana (AtFAE1), a leafy cotyledon 2 gene promoter from Arabidopsis thaliana (AtLEC2), a leafy cotyledon 1 gene promoter from Zea mays (ZmLEC1), an aspartic protease 1 gene promoter from Oryza sativa or Zea mays (OsASP1 or ZmASP1), an oleosin (OLE) gene promoter from Zea mays, a legumin 1A gene promoter from Zea mays (ZmLEG1A), an AGAMOUS gene promoter from Zea mays (ZmZAG1), or a CLAVATA 1 gene promoter from Zea mays (ZmCLV1). In some embodiments, the plant growth and/or development nucleic acid/gene is a HD-Zip transcription factor, such as the REVOLUTA (REV) gene. In some further embodiments, the REV gene is from Arabidopsis thaliana, Zea mays, Brassica napus, camelina, soybean, rice, sorghum, or wheat. For example, the transformed cell, tissue culture or plant part comprises a mutant Arabidopsis thaliana REV gene, in which the Revoluta coding sequence (SEQ ID NO. 8) is mutated such that a Thymidine at nucleotide 567 is changed to an Adenine and a Guanidine at nucleotide 570 is changed to an Adenine; or comprises a Zea mays mutant REV gene, in which the Revoluta coding sequence (Zm RLD1, SEQ ID NO. 10) is mutated such that a Thymidine at nucleotide 579 is changed to an Adenine and a Guanidine nucleotide 582 is changed to an Adenine; or comprises a mutant Arabidopsis thaliana REV gene, in which the Revoluta coding sequence is mutated such that a stop codon is encoded at amino acid residue positions 11 and 18.

The present methods and compositions increase seed size and/or seed number in plants. In some embodiments, the present methods and compositions relate to the use of a modified growth and/or development regulatory nucleic acid/gene that is over-expressed in a plant. In particular, the present methods and compositions relate to the use of a miRNA-resistant growth and/or development regulatory nucleic acid/gene, or a growth and/or development regulatory nucleic acid/gene comprising one or more early stop codons under the control of an appropriate plant promoter. In some embodiments, the plant promoter can be an embryo-specific promoter, an endosperm-specific promoter, or an ear-specific promoter to provide for the over expression of the gene and/or a protein encoded by the gene in the developing seed of a plant. In some embodiments, the embryo specific promoter is an early phase-specific embryo promoter. In some embodiments, the plant is a transgenic plant, and the modified growth and/or development regulatory gene is a transgene in the transgenic plant. Over expression of the modified gene in a plant, for example, during an early stage of seed development in a plant provides for increased seed production and/or increased seed size in the transgenic plant when compared with the wild-type plant.

In a particular embodiment the miRNA binding site of the REVOLUTA (REV) nucleic acid/gene is mutated to significantly reduce or eliminate binding and control by miRNA. In some other embodiments, a growth and/or development regulatory nucleic acid/gene is mutated to have one or more early stop codons. The mutated transgene can be operatively associated with an embryo-specific promoter, an endosperm-specific promoter, or an ear-specific promoter to provide for the over expression of REV protein in a developing seed of a transgenic plant. Over expression of REV, for example, during an early stage of seed development surprisingly results in increased seed size and/or increased seed numbers in the transgenic plant without the detrimental side effects that had been seen when REV was over expressed throughout the plant using a constitutive promoter as reported in WO/2001/033944 and U.S. Pat. No. 7,056,739, each of which is incorporated by reference in its entirety. In addition, such early stage seed-specific expression of REV results in a statistically significant increase in seed size and increased seed number as reported in WO/2007/079353 and US Published Patent Application No. US 2008-0263727, each of which is incorporated by reference in its entirety.

The modified growth and/or development nucleic acids/genes of the present invention can be expressed at any appropriate stages in any appropriate parts in a plant, so long as the expression leads to increased seed number and/or seed size in the plant. In some embodiments, the nucleic acid/gene is over-expressed in a seed during early embryo development. In some embodiments, the nucleic acid/gene is over-expressed in an embryo, an endosperm, or an ear (female inflorescence). In some other embodiments, the nucleic acid/gene is over-expressed in one or more plant parts other than a seed during any desired developmental stage.

In some embodiments, the method comprises: a) identifying at least one mutant plant growth and/or development gene comprising one or more mutations at an microRNA binding site, or comprising one or more early stop codons; b) constructing an expression construct comprising the mutated plant growth and/or development gene; c) transforming a plant cell with the expression vector of step (b); d) selecting for a plant cell comprising the expression vector of step (b); e) regenerating the plant from the plant cell comprising the expression vector of step (b); and f) growing the plant of step (e) to obtain a mature plant with a phenotype of having an increased seed yield and/or seed size as compared with a wild-type plant or a plant which does not comprise the mutated plant growth and/or development gene(s). The mutant growth and/or development related gene can be obtained by mutating a wild type growth and/or development related gene. Methods of mutating genes and screening such mutation are well known to one skilled in the art. In some other embodiments, the mutant growth and/or development related gene occurred naturally without artificial mutagenesis method. Such mutant can be screened and isolated. In particular, the method comprises expressing a miRNA-resistant growth and/or development related gene, or a growth and/or development related gene having one or more early stop codons, in the seed under the control of an embryo-specific promoter, an endosperm-specific promoter, or an ear-specific promoter. In some embodiments, the embryo specific promoter is an early phase-specific embryo promoter. In some embodiments, the embryo-specific promoter, the endosperm-specific promoter, or the ear-specific promoter can be heterologous or homologous to the plant. In certain embodiments of the present invention the promoter is an early phase-specific embryo promoter associated with an amino acid permease gene, such as AAP1, an oleate 12-hydroxylase:desaturase gene, a 2S2 albumin gene, a fatty acid elongase gene, such as FAE1, a leafy cotyledon gene, an oleosin gene, or an aspartic protease gene; the endosperm-specific promoter can be a legumin 1A (LEG1A) gene; and the ear-specific promoter can be an AGAMOUS gene or a CLAVATA 1 gene. Particular promoters useful in the present invention include an AAP1 promoter from Arabidopsis thaliana (AtAAP1), or functional part thereof, an oleate 12-hydroxylase:desaturase promoter from Lesquerella fendleri (LFAH12), or functional part thereof, a 2S2 promoter from Arabidopsis thaliana (At2S2), or functional part thereof, a fatty acid elongase promoter from Arabidopsis thaliana (AtFAE1), or functional part thereof, a leafy cotyledon 2 promoter from Arabidopsis thaliana (AtLEC2), or functional part thereof, a leafy cotyledon 1 promoter from Zea mays (ZmLEC1), or functional part thereof, an aspartic protease 1 promoter from Oryza sativa or Zea mays (OsASP1 or ZmASP1), or functional part thereof, an oleosin (OLE) promoter from Zea mays, or functional part thereof, a legumin 1A promoter from Zea mays (ZmLEG1A), or functional part thereof, an AGAMOUS promoter from Zea mays (ZmZAG1), or functional part thereof, or a CLAVATA 1 promoter from Zea mays (ZmCLV1), or functional part thereof.

In another embodiment a modified REV gene is operatively associated with an embryo-specific promoter, for example, an early phase embryo-specific promoter. In this method, the modified REV gene is over-expressed in the early development of the seed and leads to an increase in seed size and seed number as compared with a wild-type plant. In another embodiment a modified REV gene is operatively associated with an endosperm-specific promoter, or an ear-specific promoter. In this method, the modified REV gene is over-expressed in the endosperm or ear and leads to an increase in seed size and seed number as compared with a wild-type plant.

The methods and compositions disclosed herein can be used to increase the seed size and/or seed number in plants that are characterized as a monocot or a dicot. The methods and compositions of the present invention can be used to increase the seed size and/or seed number in plants that are members of the Brassicaceae, Cruciferae, Gramineae, Malvaceae, or Leguminosae-Papilionoideae families. Some exemplary plants of interest for use of the methods and compositions of the present invention include, for example, canola, corn, camelina, cotton, alfalfa, soybean, wheat, rice, barley, and the like.

Also provided are genetic constructs comprising a nucleic acid sequence for a gene associated with plant growth and/or development which is modified and operatively linked to one or more control sequences wherein the one or more control sequences are capable of promoting expression of the gene in a plant, for example, during embryo development. The genetic constructs disclosed herein can comprise a control sequence including an embryo-specific promoter, an endosperm-specific promoter, or an ear-specific promoter. In some embodiments, the embryo specific promoter is an early phase-specific embryo promoter. The early phase specific embryo promoters can include, for example, the promoter associated with an amino acid permease gene (AAP1), an oleate 12-hydroxylase:desaturase gene, a 2S2 albumin gene (2S2), a fatty acid elongase gene (FAE1), a leafy cotyledon gene (LEC2), a leafy cotyledon 1 (LEC1) gene, an aspartic protease 1 gene (ASP1), or an oleosin gene. A typical genetic construct comprises the AAP1 gene promoter from Arabidopsis thaliana (SEQ ID NO: 17), or functional part thereof, the oleate 12-hydroxylase:desaturase gene promoter from Lesquerella fendleri (LFAH12, SEQ ID NO: 14), or functional part thereof, the 2S2 gene promoter from Arabidopsis thaliana, or functional part thereof, the fatty acid elongase gene promoter from Arabidopsis thaliana, or functional part thereof, the leafy cotyledon gene 2 promoter from Arabidopsis thaliana (SEQ ID NO: 16), or functional part thereof, the leafy cotyledon 1 promoter from Zea mays (ZmLEC1), or functional part thereof, the aspartic protease 1 gene promoter from Oryza sativa or Zea mays (OsAsp 1; ZmAsp 1), or functional part thereof, or the oleosin gene promoter from Zea mays (ZmOLE, SEQ ID NO: 34), or functional part thereof. The endosperm-specific promoter can be the legumin 1A gene promoter from Zea mays (ZmLEG1A, SEQ ID NO: 35), or functional part thereof. The ear-specific promoter can be the ZAG1 gene promoter from Zea mays (SEQ ID NO: 36), or functional part thereof, or the CLAVATA 1 promoter from Zea mays (ZmCLV1), or functional part thereof. In some embodiments, the genetic constructs of the present invention comprise an embryo-specific promoter, an endosperm-specific promoter, or an ear-specific promoter operatively associated with a miRNA resistant REV gene or a gene having one or more early stop codons from Arabidopsis. The genetic constructs can also include an operatively associated polyA sequence. Non-limiting exemplary sequences of promoters associated with AAP1, 2S2, FAE1, LEC2 and LFAH12 are described in WO/2007/079353; non-limiting exemplary sequences of promoters associated with Oryza sativa aspartic protease 1 are described in Bi et al. (Plant Cell Physiol, 2005, 46(1): 87-98); non-limiting exemplary sequences of promoters associated with corn oleosin gene are described in WO/1999/064579; non-limiting exemplary sequences of promoters associated with corn legumin gene are described in US Patent Publication No. 20060130184; and non-limiting exemplary sequences of promoters associated with corn AGAMOUS (ZAG1) gene are described in Schmidt et al. (Plant Cell, 1993 July; 5(7):729-37), each of which is incorporated by reference in its entirety. One skilled in the art would be able to determine and use a functional partial promoter sequence of the promoter sequences described above while still keep desired promoter activity. For example, one may use truncated version of the promoters associated with AAP1, 2S2, FAE1, LEC2, LFAH12, LEC1, ASP1, oleosin, LEG1A, or AGAMOUS or CLV1 in the present invention while still be able to obtain transgenic plants with some increased seed yield and/or seed size compared to wild type plants.

Methods for the production of a transgenic plant having increased seed size and/or seed number are also provided, wherein the methods comprise introducing into a plant or into a plant cell, a genetic construct as set forth above and cultivating the plant or plant cell comprising the genetic construct under conditions promoting regeneration and mature plant growth. Typically, the methods produce a transgenic plant having increased seed size and/or seed number when compared to the corresponding wild-type plant. Transgenic plants comprising the genetic constructs can be monocotyledonous or dicotyledonous plants, particularly where the monocotyledonous plant is a member of the Gramineae family. Some exemplary plants from the Gramineae family include rice, oat, corn, or wheat. Additionally, transgenic plants described herein are plants of the Brassicaceae (Cruciferae), Malvaceae, or Leguminosae-Papilionoideae families. In some embodiments, the transgenic plant is soybean, cotton, camelina, alfalfa, rice or canola.

The present disclosure also provides methods for selecting for a nucleic acid/gene that increases plant yield having one or more modifications when functionally associated with an embryo-specific promoter, an endosperm-specific promoter, or an ear-specific promoter; wherein the methods comprise constructing an expression vector comprising a nucleic acid/gene associated with plant growth and/or development having a mutated miRNA binding site, or one or more early stop codons functionally associated with an embryo-specific promoter, an endosperm-specific promoter, or an ear-specific promoter, transfecting a plant cell with the expression vector to form a transgenic plant; growing the transgenic plant and selecting those transgenic plants that have an increased yield. In some embodiments, the embryo specific promoter is an early phase-specific embryo promoter. The modified nucleic acids/genes that produce a transgenic plant with increased yield are selected for further development of additional transgenic plants. Genes that can be used in the present method include, for example, but are not limited to HD-Zip transcription factors (REVOLUTA (REV), PHABULOSA (PHB), PHAVOLUTA (PHV), ATHB8, CORONA (ATHB 15), and the like), NAC-containing transcription factors (for example, NAC1, CUC1, CUC2, and the like), BHLH transcription factors (including for example, TCP2, TCP3, TCP4, TCP10, TCP24, and the like), MYB transcription factors (for example, MYB33, MYB65, GAMYB, and the like), APETALA2-like transcription factors (for example, AP2, TOE1, TOE2, TOE3, GL15, and the like), SBP-like transcription factors (for example, SPL3, SPL4, SPL5, and the like), SCL transcription factors (for example, SCL6-II, SCL6-III, and the like), ARF transcription factors (for example, ARF6, ARF10, ARF16, ARF17, ARF18, and the like), F-box protein (for example, TIR1, and the like), homologs, orthoglogs, or variants thereof. In one particular embodiment the REV gene encodes a polypeptide comprising the full or partial REV from Arabidopsis thaliana (e.g., SEQ ID NO: 1, encoded by SEQ ID NO: 8), Brassica napus, camelina, soybean, wheat, rice (e.g., OsREV1, SEQ ID NO: 2, encoded by SEQ ID NO: 38; OsREV2, SEQ ID NO: 3, encoded by SEQ ID NO: 39, or TGI OsREV2, SEQ ID NO: 40, encoded by SEQ ID NO: 41), corn (e.g., ZmRLD1, SEQ ID NO: 12, encoded by SEQ ID NO: 10; or ZmRLD2, SEQ ID NO: 4, encoded by SEQ ID NO: 5), tomato (e.g., SEQ ID NO: 7) or sorghum, which are miRNA-resistant, or have one or more early stop codons. In some embodiments, the REV gene can encode a biologically active variant derived from the REV in Arabidopsis thaliana, Brassica napus, camelina, soybean, wheat, rice, corn, or sorghum, with at least 60%, at least 65%, at least 70%, at least 75%, at least 80%, at least 85%, at least 90%, at least 95%, at least 96%, at least 97%, at least 98% or at least 99% identity. In some embodiments, the REV gene can encode at least 5 amino acids, at least 10 amino acids, at least 20 amino acids, at least 30 amino acids, at least 40 amino acids, at least 50 amino acids, at least 60 amino acids, at least 70 amino acids, at least 80 amino acids, at least 90 amino acids, at least 100 amino acids, at least 150 amino acids, at least 200 amino acids, at least 300 amino acids, at least 400 amino acids, or more of the REV from Arabidopsis thaliana, Brassica napus, camelina, soybean, wheat, rice, corn, tomato, or sorghum. In another embodiment, the REV gene encodes a chimeric fusion polypeptide derived from the REV of Arabidopsis thaliana, Brassica napus, camelina, soybean, wheat, rice, corn, tomato, and/or sorghum. For example, the chimeric fusion polypeptide can comprise two or more heterologous REV proteins or parts thereof which are linked into a single macromolecule.

### DESCRIPTION OF THE TEXT FILE SUBMITTED ELECTRONICALLY

The contents of the text file submitted electronically are incorporated herein by reference in their entirety: A computer readable format copy of the Sequence Listing (filename: TARG01101US.txt, date recorded: Aug. 24, 2010, file size 130 kilobytes).

### DETAILED DESCRIPTION

Unless defined otherwise, all technical and scientific terms used herein have the same meaning as commonly understood by one of ordinary skill in the art to which the compositions and methods described herein belong. Although any methods and materials similar to those described herein can be used in the practice or testing of the present methods and materials, only exemplary methods and materials are described. For purposes of the present disclosure, the following terms are defined below.

The terms “a,” “an,” and “the” as used herein include plural referents, unless the context clearly indicates otherwise.

siRNAs were first discovered in plants (Hamilton and Baulcombe, Science 286:950-952, 1999; Llave et al., Plant Cell. 14:1605-1619, 2002) and are the prevalent small RNAs in Arabidopsis. siRNAs have roles in defense against viruses, suppression of expression from transgenes or transposons, establishment of heterochromatin, and post-transcriptional regulation of mRNAs.

miRNAs are small (20-24 nt) RNA molecules derived from non-coding miRNA genes found in many organisms (Lee et al., Cell 75:843-854 1993; Wightman et al., Cell 75:855-862, 1993; Reinhart et al., Genes Dev. 16:1616-1626, 2002). miRNAs base-pair with target mRNA sequences in their miRNA binding sites and this binding leads to the down regulation of target mRNA expression. The first case of miRNA regulation was discovered in Caenorhabditis elegans (Lee et al., Cell 75:843-854, 1993; Wightman et al., Cell 75:855-862, 1993), and since that time, many more miRNAs have been found in diverse eukaryotes, with the exception of Saccharomyces cerevisiae. Revoluta and the other four members of the HD-Zip III transcription factor family (Phavoluta (Athb-9), Phabulosa (Athb-14), Corona (Athb-15), and Athb-8) have microRNA (miRNA) binding sites in their START (sterol lipid binding) domains that are complementary to miRNAs 165 and 166 in the Arabidopsis genome. The evolutionarily conserved miRNAs are classified into gene families. Thus there are two miRNA 165 (a and b) and seven miRNA 166 (a-g) genes in the Arabidopsis genome (Reinhart et al., Genes Dev. 16:1616-1626, 2002, incorporated by reference in its entirety) that regulate the HD-Zip III transcription factor family members. A number of studies done in recent years have supported the idea that class III HD-Zip transcription factor messenger RNAs (mRNAs) are repressed in a spatially-specific manner by miRNA 165/166 and that this repression is essential for normal adaxial/abaxial fate specification, development of axillary shoot apical meristems (SAMs), or vascular development (McConnell et al., Development 125:2935-2942, 1998; McConnell et al., Nature 411:709-713, 2001; Emery et al., Curr. Biol. 13:1768-1774, 2003; Juarez et al., Nature 428:84-88, 2004; Zhong and Ye, Plant Cell Physiol. 45:369-385, 2004; Kim et al., Plant J. 42:84-94, 2005; Ochando et al., Plant Physiol. 141:607-619, 2006; Zhou et al., Plant Cell Physiol. 48:391-404, 2007; Ochando et al., Int. J. Dev. Biol. 52:953-961, 2008)). Studies have also been done, both in vivo and in vitro, to show that HD-Zip III mRNAs are cleaved in the presence of miRNA 165/166 and that this cleavage is dependent upon the miRNA binding site sequence (Tang et al., Genes Dev. 17:49-63, 2003; Floyd and Bowman, Nature 428:485-486, 2004; Zhong and Ye, Plant Cell Physiol. 45:369-385, 2004; Kim et al., Plant J. 42:84-94, 2005), each of which is herein incorporated by reference in its entirely.

siRNA and miRNA are chemically and functionally similar. Both are short non-coding RNAs (20-24 nucleotides (nt) in length) whose function is to repress the expression of defined target genes in animals and plants. Both RNA species are generated from longer precursor molecules by a Dicer-like (DCL) ribonuclease and get incorporated into ribonucleoprotein silencing complexes that effect repression of target mRNAs via base pairing of the small RNA and its target mRNA. The silencing complexes require the activity of Argonaute proteins. Repression may occur by cleavage of the target mRNA or inhibition of translation (post-transcriptional regulation) or by methylation of the target gene (transcriptional regulation) (Chen, Science 303:2022-2025, 2004; Bao et al., Dev. Cell. 7:653-662, 2004).

However, there are fundamental differences between siRNAs and miRNAs. siRNAs are derived from mRNAs, transposons, heterochromatic DNA, or viruses, but miRNAs originate from distinct loci within a plant's genome. The difference in origin of these small RNAs also defines their different targets. siRNAs usually target sequences from which they were derived, whereas miRNAs target a broad array of sequences that are unrelated to the miRNA loci. The biogenesis of the siRNA involves processing of a siRNA duplex from a long double-stranded RNA precursor, while that of miRNA involves processing of a miRNA duplex from a longer imperfect stem-loop precursor. Processing is usually performed by a ribonuclease. DCL3 or DCL4 typically processes siRNA, while DCL1 processes miRNA. Generation of siRNA require RNA-dependent RNA polymerase, while generation of miRNA does not.

Revoluta (REV) and the other four members of the HD-Zip III family (Phavoluta (Athb-9), Phabulosa (Athb-14), Corona (Athb-15), and Athb-8) have miRNA binding sites in their START (sterol lipid binding) domains that are complementary to the miRNAs designated 165 and 166. In plants there is a high level of complementarity between a miRNA and its target mRNA. Thus, it is not surprising that both the miRNA binding sites (Floyd and Bowman, Nature 428:485-486, 2004) and the miRNA sequences themselves are highly conserved among diverse plants (Rhoades et al., Cell 110:513-520, 2002; Bonnet et al., Proc. Natl. Acad. Sci. USA, 101:11511-11516, 2004). The evolutionarily conserved miRNAs are classified into gene families. Thus there are two miRNA 165 (a, b) and seven miRNA 166 (a-g) genes in the Arabidopsis genome (Reinhart et al., Genes Dev. 16:1616-1626, 2002) that regulate the HD-Zip III family members. By in situ experiments, REV is known to localize to the apical region of globular embryos and then concentrate in the adaxial regions of the cotyledons and in the vasculature of the hypocotyl in later embryo development (Otsuga et al., Plant J. 25:223-236, 2001; Emery et al., Curr. Biol. 13:1768-1774, 2003; Juarez et al., Nature 428:84-88, 2004; Williams et al., Development 132:3657-3668, 2005).

Phenotypic studies on class III HD-Zip mRNAs have commonly focused on two types of mutants: i) miRNA-resistant mutants, which contain a transgene or an endogenous gene mutated in the miRNA binding site, and ii) miRNA overexpressors, which over-express a miRNA through activation tagging or by a transgene. Mutations in the miRNA binding site of class III HD-Zip transcription factor genes give gain-of-function mutants that display, for example, adaxialized leaves and stems (McConnell and Barton, Development 125:2935-2942, 1998; McConnell et al., Nature 411:709-713, 2001; Emery et al., Curr. Biol. 13:1768-1774, 2003; Juarez et al., Nature 428:84-88, 2004, Zhong and Ye, Plant Cell Physiol. 45:369-385, 2004), ectopic development of axillary SAMs (McConnell and Barton, Development 125:2935-2942, 1998; McConnell et al., Nature 411:709-713, 2001), or poorly developed vascular tissues (Kim et al., Plant J. 42:84-94, 2005).

The miRNA binding site mutations appear to affect plant function at the nucleotide level, since mutations within the site that do not change the amino acid sequence still give the same phenotypes (Emery et al., Curr. Biol. 13:1768-1774, 2003; Mallory et al., EMBO J. 23(16):3356-3364, 2004). These studies thus showed that the HD-Zip III family members were important for polarity establishment, meristem function, and vascular development and that regulation of these genes at the RNA level was important for these functions (McConnell et al., Development 125:2935-2942, 1998; McConnell et al., Nature 411:709-713, 2001; Emery et al., Curr. Biol. 13:1768-1774, 2003; Juarez et al., Nature 428:84-88, 2004; Zhong and Ye, Plant Cell Physiol. 45:369-385, 2004; Kim et al., Plant J. 42:84-94, 2005; Ochando et al., Plant Physiol. 141:607-619, 2006; Zhou et al., Plant Cell Physiol. 48:391-404, 2007; Ochando et al., Int. J. Dev. Biol. 52:953-961, 2008)). Juarez and coworkers (Nature 428:84-88, 2004) showed by in situ hybridization analysis that the corn homolog of REV (RLD1) and Zm miRNA 166a have complementary expression patterns in leaf primordia. In the ZmREV miRNA binding site mutant, Rld-O, Rld1 mRNA was misexpressed in a region below the incipient leaf where miRNA 166a localizes, suggesting that Zm miRNA 166a normally suppresses expression of wild type RLD1 in this region. Similarly, McConnell and coworkers (Nature 411:709-713, 2001) observed that in a miRNA-resistant Phabulosa mutant, the Phabulosa mRNA had spread beyond its normal adaxial location in leaves and had accumulated in the abaxial region. Conversely, over expressing the miRNA 165/166 gives phenotypes resembling those of loss-of-function HD-Zip III transcription factor mutants (Kim et al., Plant J. 42:84-94, 2005; Zhou et al., Plant Cell Physiol. 48:391-404, 2007). Over expression of miRNA 165a causes repression of all five HD-Zip III mRNAs and yields plants that cannot form shoot apical meristems, are disturbed in organ polarity and vascular development, and possess fewer interfasicular fibers (Zhou et al., Plant Cell Physiol. 48:391-404, 2007). An excess of miRNA 166a represses the five HD-Zip III mRNAs to varying degrees and yields dwarf plants with fasciated stems, disrupted vascular patterning, enlarged meristems and short carpels (Kim et al., Plant J. 42:84-94, 2005).

Studies have also been done, both in vivo and in vitro, to show that HD-Zip III mRNAs are cleaved in the presence of miRNA 165/166 and that this cleavage is dependent upon the miRNA binding site sequence. Tang et al. (Genes Dev. 17:49-63, 2003) have shown that Phavoluta (PHV) and Phabulosa (PHB) mRNAs can be cleaved in an in vitro wheat germ extract system and that this cleavage is dependent upon miRNA 166. 5′ RACE experiments have also shown that REV mRNA is cleaved in vivo at a specific position within the miRNA binding site (Floyd and Bowman, Nature 428:485-486, 2004, Zhong and Ye, Plant Cell Physiol. 45:369-385, 2004) and that this cleavage is abolished in the miRNA-resistant REV mutant avb-1. Kim et al. (Plant J. 42:84-94, 2005) demonstrated using a Nicotiana benthamiana transient expression system that Athb-15 mRNA is cleaved in planta almost to completion in the presence of miRNA 166a and that this cleavage is abolished when Athb-15 mRNA carrying mutations in the miRNA 165/166 target sequence was used. They also showed with 5′ RACE experiments that the cleavage site in Athb-15 matched those of REV and PHV.

An mRNA surveillance system called nonsense-mediated decay (NMD) exists in all eukaryotes, including plants, to degrade native mRNAs as well as heterologous mRNAs with premature termination codons (PTCs)(Gutierrez et al., Trends Plant Sci. 4:429-438, 1999; Maquat, Nat. Rev. Mol. Cell. Biol. 5:89-99, 2004; Baker and Parker, Curr. Opin. Cell Biol. 16:293-299, 2004). Degradation of mRNAs containing nonsense mutations ensures that potentially detrimental small polypeptides do not accumulate in the organism. Van Hoof and Green (Plant J. 10:415-424, 1996) have demonstrated previously that bean phytohemagglutinin mRNA stability was dependent upon the position of the premature termination codon (PTC) within the coding region. They found that premature termination codons positioned at 20%, 40%, or 60% of the way through the coding region led to unstable mRNAs, whereas a premature termination codon situated 80% of the way through the coding region yielded mRNA that was as stable as the wild type, full length, mRNA.

Canola that over express the Arabidopsis thaliana REV (At REV) transgene in an early embryo-specific manner result in a 15% seed yield increase in replicated yield trials across multiple years. There are two straightforward interpretations of these results: i) the REV transgene functions at the protein level to cause the yield increase, or ii) the REV gene functions at the transcriptional level to cause the yield increase.

The protein model (i, above) hypothesizes that REV is transcribed from the transgene into mRNA and then subsequently translated into protein. It is the excess expression of REV protein from the transgene that is believed to lead to the yield increase, presumably by the action of excess REV protein on inhibition or activation of downstream target genes or by sequestration of other transcriptional factors.

The transcript model (ii, above) hypothesizes that REV is transcribed from the transgene into mRNA and the excess REV mRNA is seen as abnormal by the plant. The excess REV transcript can lead to the silencing of the endogenous canola REV locus by a mechanism generally called cosuppression (Jorgensen et al., Plant Mol. Biol. 31:957-973, 1996; Que and Jorgensen, Dev. Genet. 22:100-910, 1998), and therefore, the lack of REV protein somehow leads to seed yield increase. This cosuppression could be transcriptional gene silencing (for example, methylation or altered chromatin structure), post-transcriptional gene silencing through degradation of endogenous REV mRNA, or perhaps both. Alternatively, another transcript model posits that REV mRNA from the transgene serves as a miRNA sink for endogenous miRNA 165/166. Therefore, the amount of miRNA 165/166 available to suppress the endogenous REV mRNA would decrease, allowing for overexpression of endogenous REV protein.

To distinguish between the opposing protein and transcript models, the present invention generated transgenic canola events. In one embodiment the event generated a plant carrying a modified REV transgene that did not code for a full-length REV protein. For example, the Arabidopsis REV coding sequence without introns (SEQ ID NO: 8) was engineered to contain two premature translation termination codons close to the amino terminal end of the coding sequence. Introducing early stop codons into this translational REV mutant transgene would prevent expression of full length REV protein from the transgene. One would expect that such a modified REV transgene can not affect phenotypes of a plant since no functional protein would be translated. However, the inventors of the present invention surprisingly discovered that transgenic plants comprising a transgene encoding REV with premature termination codons can produce more and/or larger or heavier seeds than wild type plants.

Meanwhile, a comparison between the nucleotide sequence of the At REV miRNA binding site with that of a Brassica napus REV miRNA binding site sequence reveals only one nucleotide difference. Seventeen of the eighteen nucleotides in the At REV miRNA binding sequence that are complementary to miRNA 165/166 are identical between At REV and Bn REV. Zhou et al. (Plant Cell Physiol. 48:391-404, 2007) have demonstrated that although there is a one-nucleotide difference between miRNA 165a and the CORONA (ATHB15) miRNA binding sequence, the CORONA mRNA is still repressed upon over expression of miRNA 165a. Therefore, it is possible that in transgenic canola plants that over express the At REV transgene will be repressed by canola REV miRNA. If the At REV transgene is being down regulated by endogenous canola REV miRNA, then the maximum amount of REV protein that could be produced by the transgene may not be realized. As such, seed yield increase could potentially be much greater if there were more REV protein produced. Therefore, creating a REV miRNA mutant transgene could bypass any miRNA down regulation that might be present in a transgenic plant, such as canola, leading to more REV mRNA from the transgene and therefore, more REV protein.

However, in fact, published work with Arabidopsis plants that over express an At REV miRNA-resistant transgene, rev δmiRNA, under the direction of the At REV promoter (Emery et al., Curr. Biol. 13:1768-1774, 2003) demonstrated that these plants exhibited problems in the specification of adaxial/abaxial cell fate in the stem and leaves. The leaves of the REV over expressing plants were adaxialized, giving trumpet-shaped organs. In addition, polarity in the rev δmiRNA stem was affected also, leading to amphivasal vascular bundles with xylem tissue surrounding phloem. Zhong and Ye (Plant Cell Physiol. 45:369-385, 2004) found similar phenotypes when examining the Arabidopsis avb-1 mutant which over expresses REV. The avb-1 mutant was found to have an amino acid replacement in the miRNA binding site. Given the typical abnormal phenotypes of plants that over express a miRNA-resistant REV gene, it was reasonable to expect a similar abnormal phenotype with early embryo-specific expression of a REV transgene mutated in the miRNA binding site. In particular, it might be predicted that the over expression may have led to polarity establishment problems during embryo development.

Nevertheless, contrary to the expectations based on the previous teachings summarized above, the presently disclosed methods and materials unexpectedly but clearly demonstrate that over expression of a miRNA-resistant REV transgene results in significant seed yield increases over their corresponding wild-types in replicated yield trials across multiple locations. In some embodiments, the miRNA-resistant REV transgene is operably linked to an embryo-specific promoter, an endosperm-specific promoter, or an ear-specific promoter. In addition, the REV miRNA mutant expression in the present invention does not lead to detrimental effects in embryo development. As such, the methods and constructs of the present disclosure provide additional means to improve the growth and yield characteristics of plants, especially those of agriculturally important crops.

Therefore, the present disclosure provides methods and compositions useful for producing plants having a significant increased seed size and/or increased seed number when compared to a wild-type plant. In some embodiments, such increases seed size and/or increased seed number may lead to increased yield.

In some embodiments, the methods and compositions are related to expressing a modified transgene of a growth and/or development related protein. In one embodiment, the modified transgene is microRNA resistant. In another embodiment, the modified transgene encodes one or more premature stop codons within the coding sequence.

For example, in some embodiments, the methods comprise identifying a mutant growth and/or development related gene comprising one or more mutations at the miRNA binding site such that the miRNA does not bind substantially, or does not bind completely to the mRNA encoding the mutant growth and/or development gene. Therefore, the mutant growth and/or development related gene comprising one or more mutations at the miRNA binding site is miRNA-resistant. The miRNA-resistant mutant growth and/or development related gene can be obtained by mutating a wild type growth and/or development related gene. Methods of mutating genes and screening such mutation are well known to one skilled in the art. In some other embodiments, the mutant growth and/or development related gene occurs naturally without artificial mutagenesis method. In some other embodiments, the mutant growth and/or development related gene was caused by transgenic mutagenesis, such as T-DNA insertion, or non-transgenic mutagenesis, such as chemical mutagenesis (e.g., ethane methyl sulfonate (EMS) mutagenesis). Such mutants can be identified, screened and isolated. Methods of identifying such mutants are well known to one skilled in the art (e.g., PCR, sequencing, gene TILLING, and more). Furthermore, the mutated growth and/or development related gene is operatively associated with an appropriate promoter in an expression plasmid and subsequently transformed into a plant or plant cell. Plants comprising the miRNA-resistant transgene having the mutated or altered miRNA binding site over express the protein involved in plant growth and/or development in an embryo, an endosperm, or an ear of a plant and the mature transgenic plants demonstrate a significant increase in seed size and/or an increased number of seeds as compared with wild-type. In some embodiments, the growth and/or development related gene is a REVOLUTA gene. For example, the microRNA binding site of the REVOLUTA (REV) gene was mutated such that a REV specific miRNA, miRNA 165/166, is not able to bind and therefore, the REV transgene is miRNA-resistant. In addition, the REV transgene in the methods described herein is under the regulation of a promoter that initiates expression during embryo development, for example, particularly initiates expression during early phase-specific embryo development. Unexpectedly, the mutation of the microRNA binding site to form a miRNA-resistant REV transgene and over expression of the REV transgene in early stage embryo development did not result in abnormalities in abaxial/adaxial leaf and/or stem development, but instead resulted in transgenic plants having increased seed number and/or seed size. In one embodiment, the REV gene is from Arabidopsis thaliana, Brassica napus, Zea mays, Oryza sativa, or Solanum lycopersicum.

While in some other embodiments, the present invention provides methods comprising identifying a REV transgene comprising a premature termination codon (PTC) positioned at less than 80%, 75%, 70%, 65%, 60%, 55%, 50%, 45%, 40%, 35%, 30%, 25%, 20%, 15%, 10%, 5%, 1%, or even less of the way through the coding sequence. Embryo-specific over expression of the REV transgene comprising the early termination codons (REVstop) gave an increased seed number and/or seed size in plants, which may lead to a seed yield increase in replicated yield trials across multiple locations. Without wishing to be bound by theory, the results in the present invention would suggest that the increased seed number and/or size and the increased yield increase was not due to the translation of excess REV protein but possibly to some effect of the REV transgene at the level of RNA.

Yet an RNA-based mechanism would be unexpected because of what is understood for nonsense-mediated decay (NMD) in plants as described above and by, for example, Jofuku et al., Plant Cell 1:427-435, 1989; Dickey et al., Plant Cell 6:1171-6117, 1994; Voelker et al., EMBO J. 5:3075-3082, 1986; Petracek et al., Plant J. 21:563-569, 2000. In these early studies, researchers found that PTC-containing alleles resulted in reduced abundance of their mRNAs. All these genes, moreover, did not contain introns, suggesting that NMD in plants was not dependent on introns, unlike NMD in mammals. Yet NMD could occur for intron-containing plant genes also, as seen by Isshiki et al., (Plant Physiol. 125:1388-1395, 2001). In all eukaryotes, the recognition of PTCs requires translation. Furthermore, from studies done in the budding yeast Saccharomyces cerevisiae, researchers have posited that termination codons are recognized as premature by the ribosome and NMD is elicited due to one of two cis-acting elements: i) downstream sequence elements (DSEs) or ii) abnormally long 3′ UTRs due to the altered spatial relationship between the termination codon and the poly(A) tail. In mammals, introns are recognized as the cis-acting elements required for NMD. Thus, in a plant such as canola, for example, carrying the REV transgene comprising premature termination codons, there should be no REV protein made due to the PTCs nor should there be any significant amount of REV mRNA from the transgene due to NMD.

The present invention also provides modified growth and/or development related protein coding sequences and compositions comprising the same. In some embodiments, the growth and/or development related protein is a HD-Zip III family member. For example, the growth and/or development related protein is a REVOLUTA protein. In some embodiments, the modified REV coding sequence comprises premature termination codons that provide a REV translational mutant (REVstop). In a particular embodiment, the premature termination codons in the REV translational mutant (REVstop) demonstrated herein are situated at amino acid positions 11 and 18 of the REV protein from Arabidopsis thaliana, SEQ ID NO. 1 (about 1.3 to about 2.1% of the way through the coding region of REV). Therefore, as the PTCs are less than about 25%, about 20%, about 15%, about 10%, about 5%, about 1%, or less of the way through the coding sequence for REV, the mRNA transcribed from the REV mutant transgene would be expected to be degraded and no REV protein would be made from the transgene. As such, one would expect that seed yield may not have been affected due to no appreciable REV mRNA being present and no REV protein being produced by the plant. To the contrary, the present invention provides transgenic plants comprising a transgene encoding REV with premature termination codons that produce more and/or larger seeds than wild type plants. In some other embodiments, the mutated plant growth and/or development related gene, such as the REV gene, described herein comprises nucleotide changes in the miRNA binding site. In particular, the mutations are intended to alter the miRNA binding site such that destruction of the mRNA encoding mutant REV is substantially reduced or completely inhibited. In some embodiments, the destruction of the mRNA encoding mutant REV is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 95%, about 96%, about 97%, about 98%, about 99% or more compared to the destruction of mRNA encoding wild type REV. In addition, the mutations are selected such that the amino acid sequence encoded by the mRNA is either unchanged or if changed does not substantially alter the REV activity of the produced protein. As such, the mutation can create a codon for an amino acid that would be considered a conservative or a non-conservative substitution for the amino acid residue typically found in the REV amino acid sequence at the same position, so long as the expression of such mutant gene can increase seed number and/or seed size in a plant compared to a wild type plant. In some embodiments, the mutant REV activity is about 95%, about 90%, about 85%, about 80%, about 75%, about 70%, about 65%, about 60%, about 55%, about 50%, about 45%, or less of a wild type REV, which can be measured by transcription factor activity. In a particular embodiment the mutant REV gene (SEQ ID NO: 9) comprise a T to A substitution at nucleotide 567 and a G to A substitution at nucleotide 570 in the Arabidopsis REVOLUTA coding sequence (wild type REV, SEQ ID NO: 8), while the mutant REV gene still encodes the wild type Arabidopsis REV protein (SEQ ID NO: 1). In another embodiment, the mutant REV gene (SEQ ID NO: 11) comprise a T to A substitution at nucleotide 579 and a G to A substitution at nucleotide 582 in the Zea mays REVOLUTA coding sequence (ZmRLD1, SEQ ID NO: 10), while the mutant REV gene still encodes the wild type corn REV protein (SEQ ID NO: 12). These nucleotide changes do not affect the amino acid sequence of the REVOLUTA protein encoded. In some embodiments, the transgene of a modified growth and/or development related protein coding sequences comprise modifications that otherwise reduce and/or interrupt the ability of miRNA regulation on the growth and/or development related protein so that when expressed in a plant, the transgene leads to increased seed size and/or seed number in a plant. Methods of reducing and/or interrupting the miRNA regulation mechanism in plant are well known to one skilled in the art. Table 1 below lists more non-limiting examples of nucleotide changes to create miRNA binding site mutations in several REV coding sequences, which can be used in the present invention. One skilled in the art would be able to create more mutations based on the teaching of the present invention.

TABLE 1
Nucleotide changes to create miRNA binding
site mutations in various REV coding sequences

| REVOLUTA sequence | Nucleotide | Amino acid |
|---|---|---|
| source | changes | changes |
| Arabidopsis thaliana REV | T567A | No |
| (wild type protein, SEQ ID | G570A | No |
| NO: 1; wild type cds, SEQ | | |
| ID NO: 8) | | |
| Arabidopsis thaliana REV | C569T | P190L |
| Zea mays RLD1 | T579A | No |
| (wild type protein, SEQ ID | G582A | No |
| NO: 12; wild type cds, SEQ | | |
| ID NO: 10) | | |
| Zea mays RLD1 | C581T | P194L |
| Brassica napus REV | T579A | No |
| (wild type protein, SEQ ID | G582A | No |
| NO: 45; wild type cds, SEQ | | |
| ID NO: 46) | | |
| Brassica napus REV | C581T | P194L |
| Glycine max REV A* | T576A | No |
| (wild type protein, SEQ ID | G579A | No |
| NO: 47; wild type cds, SEQ | | |
| ID NO: 48) | | |
| Glycine max REV B | C578T | P193L |
| (wild type protein, SEQ ID | | |
| NO: 49; wild type cds, SEQ | | |
| ID NO: 50) | | |

*For the 2 soybean REV sequences (A and B), the nucleotide changes (and numbering) for the miRNA mutations are the same whether one looks at A or B sequence.

The mutant polynucleotide of the present invention can be artificially produced by mutagenesis methods well know in the art, or the mutant growth and/or development related gene occurs naturally without artificial mutagenesis method. Subsequent to the premature stop codons, the polynucleotide will further comprise a nucleotide sequence that would encode amino acids from the growth and/or development protein if the protein were expressed. The nucleotides subsequent to the stop codons can encode amino acids up to the full length protein, but can also encode a growth and/or development protein having an insertion or deletion of one or more amino acid residues. The insertion of the premature termination codons into the coding sequence prevents translation of the amino acids encoded by the nucleotide sequence subsequent to the stop codons and a functional protein is not translated.

Further, the mutated growth and/or development related protein is operatively associated with an early embryo specific promoter in an expression plasmid. Subsequently the expression plasmid comprising the mutated gene and the promoter can be transformed into a plant or plant cell. Unpredictably the plants and or plant cells comprising the mutated transgene having the altered mRNA produce a plant that demonstrates an increase in seed size and/or an increased number of seeds. In a particular embodiment the mRNA of a REVOLUTA (REV) gene is mutated to include one or more termination codons in a REV protein, for example, to include two termination codons at amino acid residue positions that are corresponding to the amino acids 11 and 18 in the Arabidopsis REV protein (SEQ ID NO: 1). In addition, the mutated REV transgene (REVstop) in the methods disclosed herein is under the regulation of a promoter that initiates expression during embryo development, endosperm development, or ear development, for example, particularly initiates expression during early phase-specific embryo development. The REV transgene with the early termination codons (REVstop) expressed in early stage embryo development did not result in abnormalities in abaxial/adaxial leaf and/or stem development of the transgenic mature plant.

It should be noted that the present disclosure also encompasses mutants of other growth and development related genes comprising early termination codons that result in transgenic plants having an increased yield as represented by an increase in seed size and/or seed number. As used herein, a plant growth and/or development related gene is a gene that plays a role in determining growth rate, overall size, tissue size, or tissue number of a plant or plays a role in the plant developmental program leading to determination of tissue identity and morphology. Such growth and development related genes are identified when modification of their function by mutation, over expression, or suppression of expression results in altered plant growth rate, overall plant size, tissue size or number, or altered development. Plant growth and/or development related genes can exert their effects through a number of mechanisms some of which include regulation of cell cycle, plant hormone synthesis/breakdown pathways, sensitivity to plant hormones, cell wall biosynthesis, cell identity determination, and the like. In the present disclosure, the plant growth and/or development gene is mutated to comprise one or more early stop codons in the first about 20% to less than about 80% of the coding sequence.

A number of plant genes have been shown by over expression or suppression analysis to play roles in growth and/or development. Examples of some, but not all, of the genes that are known to be involved in growth and/or development and that can be used or tested in the methods of the present invention are discussed herein below. The Arabidopsis CAP gene encodes a cyclase-associated protein that is involved in Ras-cAMP signaling and regulation of the actin cytoskeleton. Over expression of CAP under a glucocorticoid-inducible promoter causes a loss of actin filaments and a reduction in the size of leaves due to reduced elongation of epidermal and mesophyll cells (Barrero et al., Annals of Botany 91:599-603, 2003). Suppression of sucrose synthase gene expression in cotton leads to reduced cell fiber length and smaller and fewer fiber cells (Yong-Ling Ruan et al., Plant Cell 15:952-964, 2003). Over expression of the rice histone deacetylase 1 gene with an ABA-inducible promoter in transgenic rice resulted in plants with an increase in growth rate and abnormal shoot and root tissue development compared to the wild-type (In-Cheol Jang et al., Plant J. 33:531-541, 2003). Suppression of E2Fc by RNAi in Arabidopsis increases proliferative activity in leaves, meristems, and pericycle cells. Cells in organs were smaller but more numerous than wild type and there was a reduced ploidy level in the leaves (del Pozo et al., Plant Cell 18:2224-2235, 2006). Suppression of the BKI gene by RNAi resulted in seedlings with increased hypocotyls lengths and over expression of BKI gave dwarf plants (Xuelu and Chory, Science 313:1118-1122, 2006). In addition, transgenic plants expressing a partially constitutive steroid receptor BRI1 have longer hypocotyls (Wang et al., Dev. Cell 8:855-865, 2005). Suppression of Argos-Like (ARL) in Arabidopsis gave smaller cotyledons, leaves and other lateral organs, while overexpression gave the opposite effect. The change in organ size can be attributed to cell size rather than to cell number (Hu et al., Plant J. 47:1-9, 2006).

Analysis of plants with mutations resulting in altered growth and/or developmental phenotypes has identified a number of genes that play roles in plant growth and development. A mutation affecting brassinosteroid hormone perception, bri1-5, results in a dwarf plant (Wang et al., Dev. Cell 8:855-865, 2005). A T-DNA insertion (a knock-out) in the Arabidopsis FATB gene encoding an acyl-acyl carrier protein thioesterase leads to reduced growth rate, reduced fresh weight and low seed viability (Bonaventure et al., Plant Cell 15:1020-1033, 2003). A loss-of-function mutation in Pepino, a putative anti-phosphatase, displayed tumor-like cell proliferation at the shoot apical meristem and produced supernumerary abnormal leaves (Haberer et al., Dev. Genes Evol. 212:542-550, 2002). The Arabidopsis RGS gene (regulator of G protein signaling) has the structure of a G-protein-coupled receptor (GPCR) and contains an RGS box. RGS proteins accelerate the deactivation of the Gα subunit and thus reduce GPCR signaling. The null rgs mutant has increased cell elongation in hypocotyls grown in the dark and increased cell production in roots grown in light (Chen et al., Science 301:1728-1731, 2003). The Arabidopsis TIP1 gene plays a role in root hair development and also in cellular growth. The tip1-2 mutant has smaller rosettes, reduced height and shorter internodes (Ryan et al., New Phytol. 138:49-58, 1998 and Hemsley et al., Plant Cell 17:2554-2563, 2005). Mutants (chromosomal rearrangement or T-DNA insertion) of the Big Brother (BB) gene that give very little or no Big Brother mRNA develop larger floral organs, more flower biomass and thicker stems. Conversely, over expression of Big Brother leads to smaller floral organs, less flower biomass, thinner stems and reduced leaf size. BB may be altering cell number (Disch et al., Curr. Biol. 16:272-279, 2006).

Further, the RHD2 gene encodes an NADPH oxidase important for accumulation of reactive-oxygen species in root hairs and the subsequent activation of calcium channels. The rhd2 mutant is defective in cell expansion of the tip growing cells of the root (Foreman et al., Nature 422:442-446, 2003). The miniature mutation in maize causes a loss in the cell wall invertase, expressed from the INCW2 gene. Cells of the mn1 mutant are smaller than wild-type and mn1 seed mutants only have 20% of the endosperm weight of wild type seeds. Expansion may be compromised in cells of the peripheral layers of the mn1 endosperm and may lead to decreased mitotic activity of these cells (Vilhar et al., Plant Physiol. 129: 23-30, 2002). A T-DNA insertion mutant of WAK2, wak2-1, has decreased cell elongation in roots. WAK2 may control cell expansion through regulation of vacuolar invertase activity. Expression of an inducible antisense of WAK2 or WAK4 in plants prevents cell elongation and produces dwarf plants (Wagner and Kohorn, Plant Cell 13:303-318, 2001, Lally et al., Plant Cell 13:1317-1331, 2001, and Kohorn et al., Plant J. 46:307-316, 2006). The Arabidopsis gene AP2 plays a role in floral organ identity and establishment of floral meristem identity. Loss-of-function mutations in AP2 gives increased seed mass compared to the wild type (Masa-Ohto et al., Proc. Nat'l. Acad. Sci. USA 102:3123-3128, 2005). teb mutants have short roots, serrated leaves, and fasciation. They show defects in cell division that may be caused by a defect in G2/M cell cycle progression (Inagaki et al., Plant Cell 18:879-892, 2006).

REVOLUTA in plants have been described previously. For example, see PCT Patent Publication NO. WO2001/033944A1, WO2007/079353A1, WO2004/063379A1, Talbert et al., “The REVOLUTA gene is necessary for apical meristem development and for limiting cell divisions in the leaves and stems of Arabidopsis thaliana.” Development. 1995 September; 121(9):2723-35; Otsuga et al., “REVOLUTA regulates meristem initiation at lateral positions”, Plant J. 2001 January; 25(2):223-36; and Prigge et al., “Class III Homeodomain-Leucine Zipper Gene Family Members Have Overlapping, Antagonistic, and Distinct Roles in Arabidopsis Development,” The Plant Cell, Vol. 17, 61-76, each of which is herein incorporated by reference in its entirety.

As used herein, a plant growth and/or development related gene is a gene that plays a role in determining growth rate, overall size, tissue size, or tissue number of a plant or plays a role in the plant developmental program leading to determination of tissue identity and morphology. Such growth and development related genes are identified when modification of their function by mutation, over expression, or suppression of expression results in altered plant growth rate, overall plant size, tissue size or number, or altered development. Plant growth and/or development related genes can exert their effects through a number of mechanisms some of which include regulation of cell cycle, plant hormone synthesis/breakdown pathways, sensitivity to plant hormones, cell wall biosynthesis, cell identity determination, and the like. The plant growth and/or development related genes suitable for use in the disclosed methods also comprise a miRNA binding site and the expression and/or activity of the gene is controlled by the binding of one or more miRNA. As such, a mutated plant growth and/or development related gene as used herein is a plant growth and/or development gene that has a change in the nucleotide sequence encoding a miRNA binding site such that the controlling miRNA does not bind significantly to its binding site. The protein encoded by the mutated plant growth and/or development gene is therefore over expressed.

A number of additional plant genes have been shown by over expression or suppression analysis to play roles in growth and/or development and through nucleotide sequence analysis to comprise a miRNA binding site. Examples of some, but not all, of the genes that are known to be involved in growth and/or development and that can be used or tested in the methods of the present disclosure are discussed herein below.

Analysis of plants with mutations resulting in altered growth and/or developmental phenotypes has identified a number of genes comprising a miRNA binding site that play roles in plant growth and development. The following table 2 reproduced from Wang et al., (in Encyclopedia of Life Sciences, John Wiley and Sons, Ltd., 2007) lists a number of examples of genes comprising miRNA binding sites and that relate to developmental and/or growth related phenotypes. The table 3 reproduced from Reinhart et al. lists miRNA isolated from Arabidopsis thaliana.

TABLE 2
Growth and/or development genes comprising miRNA binding sites.

| Developmental | | | |
|---|---|---|---|
| Events | miRNA | miRNA Target | Reference |
| Leaf | miRNA165/166 | HD-Zip TFs: PHB, PHV, | Juarez et al., Nature 428: 84-88, 2004; Mallory et al., EMBO J. |
| development, | | REV, ATHB8 | 23: 3356-3364, 2004; Zhong and Ye, Plant Cell Physiol. |
| patterning and | | | 45: 369-385, 2004 |
| polarity | miRNA164a | NAC-containing TF: | Nikovics et al., Plant Cell 18: 2929-2945, 2006 |
| | | CUC2 | |
| | miRNA319/JAW | BHLH TFs: TCP2, | Palatnik et al., Nature 425: 257-263, 2003 |
| | | TCP3; TCP4, TCP10, | |
| | | TCP24 | |
| | miRNA159 | MYB TFs: MYB33, | Millar and Gubler, 2005; Palatnik et al., Nature 425: 257-263, |
| | | MYB65 | 2003 |
| Floral identity | miRNA172 | APETALA2-like TFs: | Aukerman and Sakai, Plant Cell 15: 2730-2741, 2003; Chen, |
| and flower | | AP2, TOE1, TOE2, | Science 303: 2022-2025, 2004; Mlotshwa et al., Plant Molec. |
| development | | TOE3 | Biol. 61: 781-793, 2006; Schwab et al., 2005 |
| | miRNA164c | NAC-containing TFs: | Baker et al., Curr. Biol. 15: 303-315, 2005 |
| | | CUC1, CUC2 | |
| | miRNA159 | MYB TFs: GAMYB, | Achard et al., Development 131: 3357-3365, 2004; Millar and |
| | | MYB33, MYB65 | Gubler, Plant Cell 17: 705-721, 2005; Schwab et al., |
| | | | Developmental Cell 8: 517-527, 2005; Tsuji et al., Plant J. |
| | | | 47: 427-444, 2006 |
| Flowering time | miRNA159 | MYB TFs: GAMYB | Achard et al., Development 131: 3357-3365, 2004; Schwab et |
| | | | al., Developmental Cell 8: 517-527, 2005 |
| | miRNA172 | APETALA2-like TFs: | Aukerman and Sakai, Plant Cell 15: 2730-2741, 2003; Chen, |
| | | AP2, TOE1, TOE2, | Science 303: 2022-2025, 2004; Mlotshwa et al., Plant Molec. |
| | | TOE3 | Biol. 61: 781-793, 2006; Schwab et al., Developmental Cell |
| | | | 8: 517-527, 2005 |
| | miRNA156 | SBP-like TFs: SPL3 | Schwab et al., Developmental Cell 8: 517-527, 2005 |
| | miRNA171 | SCL TFs: SCL6-II, | Llave et al., Science 297: 2053-2056, 2002; Reinhart et al., |
| | | SCL6-III | Genes Develop. 16: 1616-1626, 2002 |
| Developmental | miRNA172 | APETALA2-like TFs: | Lauter et al., Proc. Nat'l. Acad. Sci. USA 102: 9412-9417, 2005 |
| phase transition | | GL15 | |
| | miRNA156 | SBP-like TFs: SPL3, | Luo et al., FEBS Lettrs. 580: 5111-5116, 2006; Schwab et al., |
| | | SPL4, SPL5 | Develop. Cell 8: 517-527, 2005; Wu and Poethig, Development |
| | | | 133: 3539-3547, 2006 |
| Shoot and root | miRNA164 | NAC-containing TF: | Guo et al., Plant Cell 17: 1376-1386, 2005; Laufs et al., |
| development | | CUC1, CUC2, NAC1 | Development 131: 4311-4322, 2004; Mallory et al., Curr. Biol. |
| | | | 14: 1035-1046, 2004; Rhoades et al., Cell 110: 513-520, 2002; |
| | | | Schwab et al., Develop. Cell 8: 517-527, 2005 |
| Vascular and | MiRNA166 | HD-ZIP TFs: ATHB15 | Kim et al., Plant J. 42: 84-94, 2005; Ochando et al., Plant |
| plastid | | | Physiol. 141: 607-619, 2006; Rhoades et al., Cell 110: 513-520, |
| development | | | 2002; Williams et al., Development 132: 3657-3668, 2005 |
| Hormone | miRNA159 | MYB TFs: GAMYB | Achard et al., Development 131: 3357-3365, 2004; Schwab et |
| signaling for | | | al., Develop. Cell 8: 517-527, 2005 |
| plant | miRNA160 | ARF TFs: ARF10, | Mallory et al., Plant Cell 17: 1360-1375, 2005; Rhoades et al., |
| development | | ARF16, ARF17 | Cell 110: 513-520, 2002; Wang et al., Plant Cell 17: 2204-2216, |
| | | | 2005 |
| | miRNA167 | ARF TFs: ARF6, ARF18 | Rhoades et al., Cell 110: 513-520, 2002; Ru et al., Cell Res. |
| | | | 16: 457-465, 2006; Wu et al., Development 133: 3539-3547, |
| | | | 2006 |
| | miRNA164 | NAC-containing TF: | Guo et al., Plant Cell 17: 1376-1386, 2005 |
| | | NAC1 | |
| | miRNA393 | F-box protein: TIR1 | Jones-Rhoades and Bartel, Ann. Rev. Plant Biol. 57: 19-53, |
| | | | 2004; Sunkar and Zhu, Plant Cell 16: 2001-2019, 2004 |

TABLE 3
Arabidopsis thaliana MicroRNAs

| | | miRNA | Fold- | Fold | |
|---|---|---|---|---|---|
| miRNA | | length | back | back | |
| gene | miRNA sequence | (nt) | arm | length | Chr |
| miRNA | UGACAGAAGAGAGUGAGCAC | 20-21 | 5′ | 82 | 2 |
| 156a | (SEQ ID NO: 18) | | | | |
| miRNA | | | 5′ | 80 | 4 |
| 156b | | | | | |
| miRNA | | | 5′ | 83 | 4 |
| 156c | | | | | |
| miRNA | | | 5′ | 86 | 5 |
| 156d | | | | | |
| miRNA | | | 5′ | 96 | 5 |
| 156e | | | | | |
| miRNA | | | 5′ | 90 | 5 |
| 156f | | | | | |
| miRNA | UUGACAGAAGAUAGAGAGCAC | 20-21 | 5′ | 91 | 1 |
| 157a | (SEQ ID NO: 19) | | | | |
| miRNA | | | 5′ | 91 | 1 |
| 157b | | | | | |
| miRNA | | | 5′ | 165 | 3 |
| 157c | | | | | |
| miRNA | | | 5′ | 173 | 1 |
| 157d | | | | | |
| miRNA | UCCCAAAUGUAGACAAAGCA | 20 | 3′ | 64 | 3 |
| 158 | (SEQ ID NO: 20) | | | | |
| miRNA | UUUGGAUUGAAGGGAGCUCUA | 21 | 3′ | 182 | 1 |
| 159 | (SEQ ID NO: 21) | | | | |
| miRNA | UGCCUGGCUCCCUGUAUGCCA | 21 | 5′ | 78 | 2 |
| 160a | (SEQ ID NO: 22) | | | | |
| miRNA | | | 5′ | 80 | 4 |
| 160b | | | | | |
| miRNA | | | 5′ | 81 | 5 |
| 160c | | | | | |
| miRNA | UUGAAAGUGACUACAUCGGGG | 20-21 | 5′ | 90 | 1 |
| 161 | (SEQ ID NO: 23) | | | | |
| miRNA | UCGAUAAACCUCUGCAUCCAG | 21 | 3′ | 85 | 5 |
| 162a | (SEQ ID NO: 24) | | | | |
| miRNA | | | 3′ | 88 | 5 |
| 162b | | | | | |
| miRNA | UUGAAGAGGACUUGGAACUUCGAU | 24 | 3′ | 303 | 1 |
| 163 | (SEQ ID NO: 25) | | | | |
| miRNA | UGGAGAAGCAGGGCACGUGCA | 21 | 5′ | 78 | 2 |
| 164a | (SEQ ID NO: 26) | | | | |
| miRNA | | | 5′ | 149 | 5 |
| 164b | | | | | |
| miRNA | UCGGACCAGGCUUCAUUCCCC | 20-21 | 3′ | 101 | 1 |
| 165a | (SEQ ID NO: 27) | | | | |
| miRNA | | | 3′ | 136 | 4 |
| 165b | | | | | |
| miRNA | UCGGACCAGGCUUCAUUCCCC | 21 | 3′ | 136 | 2 |
| 166a | (SEQ ID NO: 28) | | | | |
| miRNA | | | 3′ | 112 | 3 |
| 166b | | | | | |
| miRNA | | | 3′ | 108 | 5 |
| 166c | | | | | |
| miRNA | | | 3′ | 101 | 5 |
| 166d | | | | | |
| miRNA | | | 3′ | 135 | 5 |
| 166e | | | | | |
| miRNA | | | 3′ | 91 | 5 |
| 166f | | | | | |
| miRNA | | | 3′ | 90 | 5 |
| 166g | | | | | |
| miRNA | UGAAGCUGCCAGCAUGAUCUA | 21 | 5′ | 101 | 3 |
| 167a | (SEQ ID NO: 29) | | | | |
| miRNA | | | 5′ | 90 | 3 |
| 167b | | | | | |
| miRNA | UCGCUUGGUGCAGGUCGGGGA | 21 | 5′ | 104 | 4 |
| 168a | (SEQ ID NO: 30) | | | | |
| miRNA | | | 5′ | 89 | 5 |
| 168b | | | | | |
| miRNA | CAGCCAAGGAUGACUUGCCGA | 21 | 5′ | 190 | 3 |
| 169 | (SEQ ID NO: 31) | | | | |
| miRNA | UGAUUGAGCCGUGUCAAUAUC | 21 | 3′ | 64 | 5 |
| 170 | (SEQ ID NO: 32) | | | | |
| miRNA | UGAUUGAGCCGCGCCAAUAUC | 21 | 3′ | 92 | 3 |
| 171 | (SEQ ID NO: 33) | | | | |

The terms “growth and/or development gene” or “growth and/or development transgene” are used herein to mean any polynucleotide sequence that encodes or facilitates the expression and/or production of a nucleotide or protein encoded by the gene. Thus the terms “growth and/or development gene” or “growth and/or development transgene” can include sequences that flank the nucleotide and/or protein encoding sequences. For example, the sequences can include those nucleotide sequences that are protein encoding sequences (exons), intervening sequences (introns), the flanking 5′ and 3′ DNA regions that contain sequences required for normal expression of the gene (i.e., the promoter and polyA addition regions, respectively, and any enhancer sequences).

The terms “growth and/or development protein,” “growth and/or development homolog” or “growth and/or development associated ortholog” are used herein to mean a protein having the ability to regulate growth rate, overall size, tissue size, or tissue number of a plant or regulate the plant developmental program leading to determination of tissue identity and morphology (when utilized in the practice of the methods of the present disclosure) and that have an amino acid sequence that is at least about 70% identical, more typically at least about 75% identical, and more typically at least about 80% identical to the amino acid sequences for the protein.

As used herein an “embryo-specific gene” is a gene that is preferentially expressed during embryo development in a plant. For purposes of the present disclosure, embryo development begins with the first cell divisions in the zygote and continues through the late phase of embryo development (characterized by maturation, desiccation, and dormancy), and ends with the production of a mature and desiccated seed. Embryo-specific genes can be further classified as “early phase-specific” and “late phase-specific”. Early phase-specific genes are those expressed in embryos up to the end of embryo morphogenesis. Late phase-specific genes are those expressed from maturation through to production of a mature and desiccated seed. Examples of embryo-specific genes that initiate expression during early embryo development and are early phase-specific are known in the art. See for example, WO 2007/079353 and U.S. Pat. No. 5,965,793, each incorporated herein by reference. Promoters for these embryo-specific genes can be used for the expression of the growth and/or development related genes that comprise a mutated miRNA binding site. The early phase specific embryo promoters can include, for example, the promoter associated with an amino acid permease gene (AAP1), an oleate 12-hydroxylase:desaturase gene, a 2S2 albumin gene (2S2), a fatty acid elongase gene (FAE1), a leafy cotyledon 2 gene (LEC2), a leafy cotyledon 1 gene (LEC1), an aspartic protease gene (ASP), or an oleosin gene. Typical genetic constructs of the present disclosure comprise the AAP1 promoter from Arabidopsis thaliana, the oleate 12-hydroxylase:desaturase promoter from Lesquerella fendleri (LFAH12), the 2S2 gene promoter from Arabidopsis thaliana, the fatty acid elongase gene promoter from Arabidopsis thaliana, or the leafy cotyledon 2 gene promoter from Arabidopsis thaliana, the leafy cotyledon 1 gene promoter from Zea mays (ZmLEC1), the aspartic protease 1 gene promoter from Oryza sativa or Zea mays (OsASP1 or ZmASP1), or the oleosin gene promoter from Zea mays (ZmOLE).

As used herein an “endosperm-specific gene” is a gene that is preferentially expressed in the endosperm of a plant. Non-limiting examples of endosperm-specific gene include the rice glutelin GluB-1 gene, rice glutelin GluB-4 gene, prolamin gene, gliadin and hordein genes (Forde et al., Nucleic Acids Research, 1985, 13, 7327-7339), storage protein genes from a wide range of species, zein genes (Quayle and Faix, Molecular and General Genetics, 1992, 231, 369-374), and legumin 1A (LEG1A) gene.

As used herein an “ear-specific gene” is a gene that is preferentially expressed in the ear (female inflorescences) of a plant. Non-limiting examples of ear-specific genes include Zea mays ZAG1 gene (ZmZAG1) and Zea mays CLAVATA 1 gene.

A “heterologous sequence” is an oligonucleotide sequence that originates from a different species, or, if from the same species, is substantially modified from its original form. For example, a heterologous promoter operably linked to a structural gene is from a species different from that from which the structural gene was derived, or, if from the same species, is substantially modified from its original form.

The term “vector” refers to a piece of DNA, typically double-stranded, which may have inserted into it a piece of foreign DNA. The vector or replicon may be for example, of plasmid or viral origin. Vectors contain “replicon” polynucleotide sequences that facilitate the autonomous replication of the vector in a host cell. The term “replicon” in the context of this disclosure also includes polynucleotide sequence regions that target or otherwise facilitate the recombination of vector sequences into a host chromosome. In addition, while the foreign DNA may be inserted initially into, for example, a DNA virus vector, transformation of the viral vector DNA into a host cell may result in conversion of the viral DNA into a viral RNA vector molecule. Foreign DNA is defined as heterologous DNA, which is DNA not naturally found in the host cell, which, for example, replicates the vector molecule, encodes a selectable or screenable marker or transgene. The vector is used to transport the foreign or heterologous DNA into a suitable host cell. Once in the host cell, the vector can replicate independently of or coincidental with the host chromosomal DNA, and several copies of the vector and its inserted DNA can be generated. Alternatively, the vector can target insertion of the foreign or heterologous DNA into a host chromosome. In addition, the vector can also contain the necessary elements that permit transcription of the inserted DNA into an mRNA molecule or otherwise cause replication of the inserted DNA into multiple copies of RNA. Some expression vectors additionally contain sequence elements adjacent to the inserted DNA that allow translation of the mRNA into a protein molecule. Many molecules of mRNA and polypeptide encoded by the inserted DNA can thus be rapidly synthesized.

The term “transgene vector” refers to a vector that contains an inserted segment of DNA, the “transgene,” that is transcribed into mRNA or replicated as an RNA within a host cell. The term “transgene” refers not only to that portion of inserted DNA that is converted into RNA, but also those portions of the vector that are necessary for the transcription or replication of the RNA. In addition, a transgene need not necessarily comprise a polynucleotide sequence that contains an open reading frame capable of producing a protein.

The terms “transformed host cell,” “transformed,” and “transformation” refer to the introduction of DNA into a cell. The cell is termed a “host cell,” and it may be a prokaryotic or a eukaryotic cell. Typical prokaryotic host cells include various strains of E. coli. Typical eukaryotic host cells are plant cells (e.g., canola, cotton, camelina, alfalfa, soy, sugar cane, rice, oat, wheat, barley, or corn cells, and the like), yeast cells, insect cells, or animal cells. The introduced DNA is usually in the form of a vector containing an inserted piece of DNA. The introduced DNA sequence may be from the same species as the host cell or from a different species from the host cell, or it may be a hybrid DNA sequence, containing some foreign DNA and some DNA derived from the host species.

The term “plant” includes whole plants, plant organs, (e.g., leaves, stems, flowers, roots, and the like), seeds and plant cells (including tissue culture cells) and progeny of same. The class of plants which can be used in the methods of the present disclosure is generally as broad as the class of higher plants amenable to transformation techniques, including both monocotyledonous and dicotyledonous plants, as well as certain lower plants such as algae, e.g., cyanobacteria, and the like. It includes plants of a variety of ploidy levels, including polyploid, diploid, hexaploid, tetraploid, haploid, and the like.

A “heterologous sequence” is one that originates from a foreign species, or, if from the same species, is substantially modified from its original form. For example, a heterologous promoter operably linked to a structural gene is from a species different from that from which the structural gene was derived, or, if from the same species, is substantially modified from its original form.

The terms “REVOLUTA gene”, “REV', or “REVOLUTA transgene” are used herein to mean any polynucleotide sequence that encodes or facilitates the expression and/or production of a REVOLUTA protein. Thus the terms “REVOLUTA gene” or “REVOLUTA transgene” can include sequences that flank the REVOLUTA protein encoding sequences. For example, the sequences can include those nucleotide sequences that are protein encoding sequences (exons), intervening sequences (introns), the flanking 5′ and 3′ DNA regions that contain sequences required for normal expression of the REVOLUTA gene (i.e., the promoter and polyA addition regions, respectively, and any enhancer sequences). A mutated REVOLUTA gene as used herein has a change in the nucleotide sequence comprising a miRNA binding site such that the controlling miRNA does not significantly bind to its binding site. The REVOLUTA protein encoded by the mutated REVOLUTA gene is therefore over expressed.

The terms “REVOLUTA protein”, “REV”, “REVOLUTA homolog” or “REVOLUTA ortholog” are used herein to mean a protein having the ability to regulate plant cell division (when utilized in the practice of the methods of the present disclosure), a homeodomain, a leucine zipper region, and that have an amino acid sequence that is at least about 70% identical, more typically at least about 75% identical, and more typically at least about 80% identical to the amino acid sequences for REVOLUTA described in WO 01/33944 and WO04/63379 (incorporated herein by reference in its entirety). As used herein, the terms “homolog” or “homologue” refer to a nucleic acid or peptide sequence which has a common origin and functions similarly to a nucleic acid or peptide sequence from another species.

Alternatively, the terms “REVOLUTA protein”, “REV”, “REVOLUTA homolog”, or “REVOLUTA ortholog” are used herein to mean REVOLUTA proteins that are identified as distinct from non-REVOLUTA members of the HD-ZIPIII class of plant transcription factors. The REVOLUTA members of the HD-ZIPIII class of proteins are characterized by the lack or absence of a characteristic amino acid sequence insertion that is present in non-REVOLUTA HD-ZIPIII proteins between amino acid residues 143 and 144 of the REVOLUTA amino acid sequence described in FIG. 4A of WO 01/33944, incorporated herein by reference. The homeobox transcription factors from Arabidopsis thaliana designated Athb-8, Athb-9 (Phavaluta), Athb-14 (Phabulosa) and Athb-15 (Corona) are non-REVOLUTA HD-ZIPIII proteins and all have a characteristic amino acid sequence insertion between amino acids 143 and 144 of the REVOLUTA amino acid sequence. The five REVOLUTA amino acid sequences for A. thaliana, rice, and tomato, disclosed in WO 01/33944, all lack a 4 to 6 amino acid residue insertion at this location in the REVOLUTA amino acid sequence. The lack of this amino acid sequence insertion is a distinguishing and defining characteristic of REVOLUTA proteins. Alteration of the miRNA binding site of any polynucleotide sequence encoding a REVOLUTA protein as the term is used herein results in over-expression of the protein and typically a plant phenotype that includes increased seed size and/or seed number as compared with a plant comprising a wild-type REVOLUTA gene.

The term “percent identity” means the percentage of amino acids or nucleotides that occupy the same relative position when two amino acid sequences, or two nucleic acid sequences are aligned side by side using a computer program such as one identified below. The term “percent similarity” is a statistical measure of the degree of relatedness of two compared protein sequences. The percent similarity is calculated by a computer program that assigns a numerical value to each compared pair of amino acids based on chemical similarity (e.g., whether the compared amino acids are acidic, basic, hydrophilic, aromatic, and the like) and/or evolutionary distance as measured by the minimum number of base pair changes that would be required to convert a codon encoding one member of a pair of compared amino acids to a codon encoding the other member of the pair. Calculations are made after a best fit alignment of the two sequences has been made empirically by iterative comparison of all possible alignments. (See for example, Henikoff et al., Proc. Natl. Acad. Sci. USA 89:10915-10919, 1992).

The term “substantial identity” of polynucleotide sequences means that a polynucleotide comprises a sequence that has at least 60% sequence identity, typically at least 70%, more typically at least 80% and most typically at least 90%, compared to a reference sequence using the programs described below using standard parameters. One of skill will recognize that these values can be appropriately adjusted to determine corresponding identity of proteins encoded by two nucleotide sequences by taking into account codon degeneracy, amino acid similarity, reading frame positioning and the like.

Amino acid sequence identity can be determined, for example, in the following manner. The portion of the amino acid sequence of the protein encoded by the growth and/or development associated gene, e.g., REVOLUTA, can be used to search a nucleic acid sequence database, such as the GenBank® database, using the program BLASTP version 2.0.9 (Atschul et al., Nucl. Acids Res. 25:3389-3402, 1997). Sequence comparisons between two (or more) polynucleotides or polypeptides are typically performed by comparing sequences of the two sequences over a “comparison window” to identify and compare local regions of sequence similarity. A “comparison window” as used herein refers to a segment of at least about 20 contiguous positions, usually about 50 to about 200, more usually about 100 to about 150 in which a sequence can be compared to a reference sequence of the same number of contiguous positions after the two sequences are optimally aligned.

Optimal alignment of sequence for comparison can be conducted by local identity or similarity algorithms such as those described in Smith et al., Adv. Appl. Math. 2:482, 1981, by the homology alignment algorithm of Needleman et al., J. Mol. Biol. 48:443-453, 1970, by the search for similarity method of Pearson et al., Proc. Natl. Acad. Sci. USA 85:2444-2448, 1988, by computerized implementations of these algorithms (GAP, BESTFIT, FASTA, and TFASTA in the Wisconsin Genetics Software Package, Genetics Computer Group, 575 Science Drive, Madison, Wis.), or by visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng et al., J. Mol. Evol. 35:351-360, 1987. The method used is similar to the method described by Higgins et al., CABIOS 5:151-153, 1989. The program can align up to 300 sequences, each of a maximum length of 5,000 nucleotides or amino acids. The multiple alignment procedure begins with the pairwise alignment of the two most related sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their nucleotide or amino acid coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described in Altschul et al., J. Mol. Biol. 215:403-410, 1990. Software for performing BLAST analyses is publicly available through the National Center for Biotechnology Information web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as long as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and the speed of the alignment. The BLAST program uses as defaults a wordlength (W) of 11, the BLOSUM62 scoring matrix (see, Henikoff et al., Proc. Natl. Acad. Sci. USA 89:10915-10919, 1992) alignments (B) of 50, expectation (E) of 10, M=5, N=−4, and a comparison of both strands.

In addition to calculating percent sequence identity, the BLAST algorithm also performs statistical analysis of the similarity between two sequences (see e.g., Karlin et al., Proc. Natl. Acad. Sci. USA 90:5873-5877, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison test is less than about 0.1, more preferably less than about 0.01, and most preferably less than about 0.001. Additional methods and algorithms for sequence alignment and analysis of sequence similarity are well known to the skilled artisan.

In the case where the inserted polynucleotide sequence encoding a miRNA resistant growth and/or development gene is transcribed and translated to produce a functional polypeptide, one of skill will recognize that because of codon degeneracy a number of polynucleotide sequences will encode the same polypeptide. These variants are specifically covered by the terms “growth and/or development gene” and “growth and/or development transgene”, and specifically “REVOLUTA gene” and “REVOLUTA transgene”. In addition, these terms specifically include those full length sequences substantially identical with a gene sequence and that encode a protein that retains the function of the gene product, e.g., REVOLUTA. Two nucleic acid sequences or polynucleotides are said to be “identical” if the sequences of nucleotides or amino acid residues, respectively, in the two sequences is the same when aligned for maximum correspondence as described above. The term “complementary to” is used herein to mean that the complementary sequence is identical to all or a portion of a reference polynucleotide sequence.

Variations and alterations in the amino acid sequence of the growth and/or development associated gene and growth and/or development associated protein, e.g., the REVOLUTA gene and REVOLUTA protein sequences are described in WO 01/33944, incorporated herein by reference. The gene of interest, such as the REVOLUTA gene, polynucleotide or polynucleotide sequence can be isolated from or obtained from any plant species. In a particular embodiment of the present disclosure the REVOLUTA gene sequence used is that from Arabidopsis thaliana, but the REVOLUTA gene from other species of interest can also be used. For example the nucleotide sequence and amino acid sequence for REVOLUTA from corn (Zea mays) is described in WO 2004/063379 (incorporated herein by reference in its entirety), rice, tomato, soybean, camelina, and the like. As such, a growth and/or development gene from one plant can be used in another plant, a heterologous transformation, or a growth and/or development gene from a plant species can be mutated and used in transforming the same plant species, a homologous transformation. In other embodiments a growth and/or development gene from a monocot plant can be modified or mutated and used to transform another monocot plant or a growth and/or development gene from a dicot plant can be modified or mutated and used to transform another dicot plant. In still other embodiments a growth and/or development gene from a monocot plant can be modified or mutated and used to transform a dicot plant and vice versa.

The terms “biological activity”, “biologically active”, “activity”, “active”, “biological function”, “REV biological activity”, and “functionally active” refer to the ability of the protein of interest, such as REVOLUTA proteins to dimerize (or otherwise assemble into protein oligomers), or the ability to modulate or otherwise effect the dimerization of native wild-type (e.g., endogenous) REVOLUTA protein. However, the terms are also intended to encompass the ability of a protein of interest, such as the REVOLUTA proteins, to bind and/or interact with other molecules, including for example, but not by limitation, DNA containing specific nucleotide sequences in promoter regions recognized by the protein, e.g., the REVOLUTA protein, and which binding and/or interaction events(s) mediate plant cell division and ultimately confer a phenotype, or the ability to modulate or otherwise effect the binding and/or interaction of other molecules with native wild-type protein and which binding and/or interaction event(s) mediate plant cell division and ultimately confer a phenotype associated with the gene of interest. One skilled in the art would be able to produce biologically active REV variants derived the REV proteins in the present invention with one or more modification, and REV genes encoding thereof. As used herein, the term “protein modification” refers to, e.g., amino acid substitution, amino acid modification, deletion, and/or insertion, as is well understood in the art. The modification can be either conservative substitutions or non-conservative substitutions. The following table shows exemplary conservative amino acid substitutions.

TABLE 4
Conserved Amino Acid Substitutions

| | Very Highly - | Highly Conserved | |
|---|---|---|---|
| Original | Conserved | Substitutions (from the | Conserved Substitutions |
| Residue | Substitutions | Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

REV phenotype as used herein is intended to refer to a phenotype conferred by a REV nucleic acid or protein and particularly encompasses the characteristic wherein an increase in the seed size and/or seed number is exhibited. Typically, a REV phenotype is determined by examination of a plant over expressing REV during embryo development, for example, during early phase-specific embryo development, where the number and size of seeds from the plant can be compared to the number and size of seeds in the corresponding tissues of a parental or wild-type plant. Plants having the REV phenotype have a statistically significant change in the number and/or size of the seeds within a representative number of plants in a plant population. In some embodiments of the present invention, the seed size of the transgenic plants of the present invention increases about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 300%, about 350%, about 400%, or more compared to a control plant, such as a wild type plant or a plant comprising a control vector (e.g., a vector does not express REV gene under the control of the promoters of the present invention). In some other embodiments, the seed number of the transgenic plants of the present invention calculated by per plant, or per acre increases about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 300%, about 350%, about 400%, or more compared to a control plant, such as a wild type plant or a plant comprising a control vector. Still in some embodiments, both the seed size, and seed number of the transgenic plants of the present invention increase compared to a control plant, such as a wild type plant or a plant comprising a control vector.

The mutated plant growth and/or development related gene, such as the REVstop transgene, of the present disclosure comprises nucleotide changes that insert early termination codons into the nucleotide sequence encoding REV. In particular, the mutations are intended to stop the production of an amino acid sequence encoding REV, but produce an RNA sequence substantially similar to the REV encoding sequence. While not being bound by a particular mechanism of action, two mechanisms are possible. First, the REV transgene comprising the early termination codon(s) (REVstop) is transcribed into mRNA and the excess REVstop mRNA could be seen as abnormal by the plant. The excess REVstop mRNA would then lead to the silencing of the endogenous REV locus by, for example, cosuppression, and therefore, the lack of REV protein would lead to seed yield increase. Alternatively, the REVstop mRNA does contain the sequence of the miRNA binding site such that the endogenous miRNA 165/166 present in the plant is likely to bind to a statistically high percentage of the mutant REVstop mRNA. As such, the amount of miRNA 165/166 available to suppress the endogenous REV mRNA would decrease, leading to a statistically significant amount of the endogenous wild-type REV mRNA not substantially bound by miRNA 165/166. This modification would allow the over expression of endogenous REV protein and the number and/or size of the seeds produced by the transgenic plant would be statistically increased. In addition, further mutations or changes in the nucleotide sequence, and therefore, the amino acid sequence can be selected such that the amino acid sequence encoded by the mRNA is either not changed or if changed does not substantially alter the REV amino acid sequence of the produced proteins. As such, the mutation can create a codon for an amino acid that would be considered a conservative substitution for the amino acid typically found in the REV amino acid sequence at the same position. In a particular embodiment of the present disclosure mutagenesis created an A to T change at nucleotide 31 and an A to T change at nucleotide 52 in the Arabidopsis REV coding sequence (SEQ ID NO: 8), resulting in the conversion of the Arginine at amino acid positions 11 and 18 of Arabidopsis REV protein (SEQ ID NO: 1) to stop codons and forming the REVstop transgene (SEQ ID NO: 42). These changes did not affect the overall amino acid sequence of REV encoded by the mRNA, except to insert early termination codons at amino acid residue positions 11 and 18.

The mutations in the coding sequence for the protein of interest can be produced by any of a variety of mutagenesis procedures. Many such procedures are known in the art, including site directed mutagenesis, oligonucleotide-directed mutagenesis, and many others. For example, site directed mutagenesis is described, e.g., in Smith (Ann. Rev. Genet. 19:423-462, 1985) and references therein, Botstein & Shortie (Science 229:1193-1201, 1985); and Carter (Biochem. J. 237:1-7, 1986). Oligonucleotide-directed mutagenesis is described, e.g., in Zoller & Smith (Nucl. Acids Res. 10:6487-6500, 1982). Mutagenesis using modified bases is described, e.g., in Kunkel (Proc. Natl. Acad. Sci. USA 82:488-492, 1985), and Taylor et al., (Nucl. Acids Res. 13: 8765-8787, 1985). Mutagenesis using gapped duplex DNA is described, e.g., in Kramer et al. (Nucl. Acids Res. 12: 9441-9460, 1984). Point mismatch mutagenesis is described, e.g., by Kramer et al. (Cell 38:879-887, 1984). Double-strand break mutagenesis is described, e.g., in Mandecki (Proc. Natl. Acad. Sci. USA 83:7177-7181, 1986), and in Arnold (Current Opinion in Biotechnology 4:450-455, 1993). Mutagenesis using repair-deficient host strains is described, e.g., in Carter et al., (Nucl. Acids Res. 13: 4431-4443, 1985). Mutagenesis by total gene synthesis is described, e.g., by Nambiar et al. (Science 223: 1299-1301, 1984). DNA shuffling is described, e.g., by Stemmer (Nature 370:389-391, 1994), and Stemmer (Proc. Natl. Acad. Sci. USA 91:10747-10751, 1994).

Many of the above methods are further described in Methods in Enzymology Volume 154, entitled “Recombinant DNA, Part E”, 1988, which also describes useful controls for trouble-shooting problems with various mutagenesis methods. Kits for mutagenesis, library construction and other diversity generation methods are also commercially available. For example, kits are available from, e.g., Amersham International plc (Piscataway, N.J.) (e.g., using the Eckstein method above), Bio/Can Scientific (Mississauga, Ontario, CANADA), Bio-Rad Laboratories (Hercules, Calif.) (e.g., using the Kunkel method described above), Boehringer Mannheim Corp. (Ridgefield, Conn.), Clonetech Laboratories of BD Biosciences (Palo Alto, Calif.), DNA Technologies (Gaithersburg, Md.), Epicentre Technologies (Madison, Wis.) (e.g., the 5 prime 3 prime kit); Genpak Inc. (Stony Brook, N.Y.), Lemargo Inc (Toronto, CANADA), Invitrogen Life Technologies (Carlsbad, Calif.), New England Biolabs (Beverly, Mass.), Pharmacia Biotech (Peapack, N.J.), Promega Corp. (Madison, Wis.), QBiogene (Carlsbad, Calif.), and Stratagene (La Jolla, Calif.) (e.g., QuickChange™ site-directed mutagenesis kit and Chameleon™ double-stranded, site-directed mutagenesis kit).

In general, a promoter suitable for being operably linked to a plant growth and/or development associated gene and expressed using the described methods of the present invention typically has greater expression in embryo and lower or no expression in other plant tissues. Of particular interest are those promoter sequences that initiate expression in embryo development, for example, during early phase-specific embryo development. An early phase-specific promoter is a promoter that initiates expression of a protein prior to day 7 after pollination (walking stick) in Arabidopsis or an equivalent stage in another plant species. Examples of promoter sequences of particular interest include a promoter for the amino acid permease gene (AAP1) (e.g., the AAP1 promoter from Arabidopsis thaliana) (Hirner et al., Plant J. 14:535-544, 1998), a promoter for the oleate 12-hydroxylase:desaturase gene (e.g., the promoter designated LFAH12 from Lesquerella fendleri) (Broun et al., Plant J. 13:201-210, 1998), a promoter for the 2S2 albumin gene (e.g., the 2S2 promoter from Arabidopsis thaliana) (Guerche et al., Plant Cell 2:469-478, 1990), a fatty acid elongase gene promoter (FAE1) (e.g., the FAE1 promoter from Arabidopsis thaliana) (Rossak et al., Plant Mol. Biol. 46:717-715, 2001), and the leafy cotyledon gene promoter (LEC2) (e.g., the LEC2 gene promoter from Arabidopsis thaliana)(Kroj et al., Development 130:6065-6073, 2003). Other early embryo-specific promoters of interest include, but are not limited to, ZmLEC1 (Zhang et al., Planta 215(2): 191-194), OsASP1 (Bi et al., Plant Cell Physiol 4691): 87-98), Seedstick (Pinyopich et al., Nature 424:85-88, 2003), Fbp7 and Fbp11 (Petunia Seedstick)(Colombo et al., Plant Cell. 9:703-715, 1997), Banyuls (Devic et al., Plant J. 19:387-398, 1999), agl-15 and agl-18 (Lehti-Shiu et al., Plant Mol. Biol. 58:89-107, 2005), Phe1 (Kohler et al., Genes Develop. 17:1540-1553, 2003), Per1 (Haslekas et al., Plant Mol. Biol. 36:833-845, 1998; Haslekas et al., Plant Mol. Biol. 53:313-326, 2003), emb175 (Cushing et al., Planta 221:424-436, 2005), L11 (Kwong et al., Plant Cell 15:5-18, 2003), Lec1 (Lotan et al., Cell 93:1195-1205, 1998), Fusca3 (Kroj et al., Development 130:6065-6073, 2003), tt12 (Debeaujon et al., Plant Cell 13:853-871, 2001), ttl6 (Nesi et al., Plant Cell 14:2463-2479, 2002), A-RZf (Zou and Taylor, Gene 196:291-295, 1997), TtG1 (Walker et al., Plant Cell 11:1337-1350, 1999; Tsuchiya et al., Plant J. 37:73-81, 2004), Tt1 (Sagasser et al., Genes Dev. 16:138-149, 2002), TT8 (Nesi et al., Plant Cell 12:1863-1878, 2000), Gea-8 (carrot) (Lin and Zimmerman, J. Exp. Botany 50:1139-1147, 1999), Knox (rice) (Postma-Haarsma et al., Plant Mol. Biol. 39:257-271, 1999), Oleosin (Plant et al., Plant Mol. Biol. 25:193-205, 1994; Keddie et al., Plant Mol. Biol. 24:327-340, 1994), ABI3 (Ng et al., Plant Mol. Biol. 54:25-38, 2004; Parcy et al., Plant Cell 6:1567-1582, 1994), and the like.

The promoters suitable for use in the present methods can be used either from the same species of plant to be transformed or can be from a heterologous species. Further, the promoter can be from the same species as for the REV transgene to be used or it can be from a heterologous species. Promoters for use in the methods of the present invention can also comprise a chimeric promoter which can include a combination of promoters that have an expression profile in common with one or more of those described above. In one embodiment of the present invention, the AAP1 gene promoter from Arabidopsis thaliana, or functional part thereof was combined with the Arabidopsis thaliana REV gene and used to construct transgenic canola (Brassica napus). Further, in an additional embodiment of the present invention the oleate 12-hydroxylase:desaturase gene promoter LFAH12 from Lesquerella fendleri, or functional part thereof was operatively linked to the Arabidopsis thaliana REV gene and used to construct transgenic canola (Brassica napus). Each of the above transgenic plants demonstrated the REV phenotype characteristic of the methods of the present invention wherein a modified REV is over expressed in early embryo development resulting in increased seed size and/or seed number. In other embodiments, a modified REV gene is operably linked to an endosperm-specific promoter (e.g., ZmLEG1A gene promoter), or an ear-specific promoter (e.g., ZmZAG1 gene promoter or ZmCLV1 gene promoter).

It should be noted that the promoters described above are only representative promoters that can be used in the methods of the present invention. Methods for identifying and characterizing promoter regions in plant genomic DNA are well known to the skilled artisan and include, for example, those described by Jordano et al., Plant Cell 1:855-866, 1989; Bustos et al., Plant Cell 1:839-854, 1989; Green et al., EMBO J. 7:4035-4044, 1988; Meier et al., Plant Cell 3:309-316, 1991; and Zhang et al., Plant Physiol. 110:1069-1079, 1996. Other type plant promoters, which include, but are not limited to, constitutive promoters, non-constitutive promoters, organ-specific promoters, cell-type specific promoters, artificial promoters, can all be used in the present invention, so long as the expression under the control of such a promoter leads to increased seed number and/or seed size, without causing any negative effects on plant development. As used herein, the term “plant promoter” refers to a promoter than can drive the expression of a gene in a plant.

Transgenic plants which express REV from the mutated sequence of the present invention during embryo development, for example, during early phase-specific embryo development, or express REV from the mutated sequence of the present invention in endosperm, or in ear (female inflorescences) can be obtained, for example, by transferring transgenic vectors (e.g., plasmids, virus, and the like) that encode an embryo promoter, an endosperm-specific promoter, or an ear-specific promoter operatively linked to a gene that encodes the mutated REVOLUTA into a plant. In some embodiments, the embryo specific promoter is an early phase-specific embryo promoter. Typically, when the vector is a plasmid the vector also includes a selectable marker gene, e.g., the neomycin phosphotransferase gene encoding resistance to kanamycin, and the like. The most common method of plant transformation is performed by cloning a target transgene into a plant transformation vector that is then transformed into Agrobacterium tumefaciens containing a helper Ti-plasmid as described in Hoeckeme et al., (Nature 303:179-181, 1983). Additional methods are described in for example, Maloney et al., Plant Cell Reports 8:238, 1989. The Agrobacterium cells containing the transgene vector can be incubated with leaf slices of the plant to be transformed as described by An et al. (Plant Physiol. 81:301-305, 1986; Hooykaas, Plant Mol. Biol. 13:327-336, 1989). Transformation of cultured plant host cells is typically accomplished through Agrobacterium tumefaciens, as described above. Cultures of host cells that do not have rigid cell membrane barriers are usually transformed using the calcium phosphate method as originally described by Graham et al. (Virology 52:546, 1978) and modified as described in Sambrook et al. (Molecular Cloning: A Laboratory Manual (2nd Ed., 1989 Cold Spring Harbor Laboratory Press, New York, N.Y.). However, other methods for introducing DNA into cells such as Polybrene (Kawai et al., Mol. Cell. Biol. 4:1172, 1984), protoplast fusion (Schaffner, Proc. Natl. Acad. Sci. USA 77:2163, 1980), electroporation (Neumann et al., EMBO J. 1:841, 1982), and direct microinjection into nuclei (Capecchi, Cell 22:479, 1980) can also be used. Transformed plant calli can be selected through the selectable marker by growing the cells on a medium containing, e.g., kanamycin, and appropriate amounts of phytohormone such as naphthalene acetic acid and benzyladenine for callus and shoot induction. The plant cells can then be regenerated and the resulting plants transferred to soil using techniques well known to those skilled in the art.

In addition to the methods described above, a large number of methods are well known in the art for transferring cloned DNA into a wide variety of plant species, including gymnosperms, angiosperms, monocots and dicots (see, e.g., Glick and Thompson, eds., Methods in Plant Molecular Biology and Biotechnology, CRC Press, Boca Raton, Fla., 1993; Vasil, Plant Mol. Biol. 25:925-937, 1994; and Komai et al., Current Opinions Plant Biol. 1:161-165, 1998 (general review); Loopstra et al., Plant Mol. Biol. 15:1-9, 1990; and Brasileiro et al., Plant Mol. Biol. 17:441-452, 1990 (transformation of trees); Eimert et al., Plant Mol. Biol. 19:485-490, 1992 (transformation of Brassica); Hiei et al., Plant J. 6:271-282, 1994; Hiei et al., Plant Mol. Biol. 35:205-218, 1997; Chan et al., Plant Mol. Biol. 22:491-506, 1993; U.S. Pat. Nos. 5,516,668 and 5,824,857 (rice transformation); and U.S. Pat. Nos. 5,955,362 (wheat transformation); 5,969,213 (monocot transformation); 5,780,798 (corn transformation); 5,959,179 and 5,914,451 (soybean transformation). Representative examples include electroporation-facilitated DNA uptake by protoplasts (Rhodes et al., Science 240:204-207, 1988; Bates, Meth. Mol. Biol. 111:359-366, 1999; D'Halluin et al., Meth. Mol. Biol. 111:367-373, 1999; U.S. Pat. No. 5,914,451); treatment of protoplasts with polyethylene glycol (Lyznik et al., Plant Mol. Biol. 13:151-161, 1989; Datta et al., Meth. Mol. Biol. 111:335-334, 1999); and bombardment of cells with DNA laden microprojectiles (Klein et al., Plant Physiol. 91:440-444, 1989; Boynton et al., Science 240:1534-1538, 1988; Register et al., Plant Mol. Biol. 25:951-961, 1994; Barcelo et al., Plant J. 5:583-592, 1994; Vasil et al., Meth. Mol. Biol. 111:349-358, 1999; Christou, Plant Mol. Biol. 35:197-203, 1997; Finer et al., Curr. Top. Microbiol. Immunol. 240:59-80, 1999). Additionally, plant transformation strategies and techniques are reviewed in Birch, Ann. Rev. Plant Phys. Plant Mol. Biol. 48:297, 1997; Forester et al., Exp. Agric. 33:15-33, 1997. Minor variations make these technologies applicable to a broad range of plant species.

In the case of monocot transformation, particle bombardment is typically the method of choice. However, monocots such as maize can also be transformed by using Agrobacterium transformation methods as described in U.S. Pat. No. 5,591,616. Another method to effect monocot transformation, e.g., corn, cells from embryogenic suspension cultures are mixed with a suspension of fibers (5% w/v, Silar SC-9 whiskers) and plasmid DNA and which is then placed in a multiple sample head on a vortex mixer or horizontally in the holder of a dental amalgam mixer. Transformation can then be carried out by mixing at full speed or shaking at fixed speed for 1 second. This process results in the production of cell populations out of which stable transformants can be selected. Plants are regenerated from the stably transformed callus and these plants and their progeny can be shown by Southern hybridization analysis to be transgenic. The principal advantages of the approach are its simplicity and low cost. Unlike particle bombardment, expensive equipment and supplies are not required. The use of whiskers for the transformation of plant cells, particularly maize, is described in, for example, U.S. Pat. No. 5,464,765.

U.S. Pat. No. 5,968,830 describes methods of transforming and regenerating soybean. U.S. Pat. No. 5,969,215 describes transformation techniques for producing transformed Beta vulgaris plants, such as the sugar beet.

Each of the above transformation techniques has advantages and disadvantages. In each of the techniques, DNA from a plasmid is genetically engineered such that it contains not only the gene of interest, but also selectable and screenable marker genes. A selectable marker gene is used to select only those cells that have integrated copies of the plasmid (the construction is such that the gene of interest and the selectable and screenable genes are transferred as a unit). The screenable gene provides another check for the successful culturing of only those cells carrying the genes of interest.

Traditional Agrobacterium transformation with antibiotic resistance selectable markers can be problematical because of public opposition that such plants pose an undue risk of spreading antibiotic tolerance to animals and humans. Such antibiotic markers can be eliminated from plants by transforming plants using the Agrobacterium techniques similar to those described in U.S. Pat. No. 5,731,179. Antibiotic resistance issues can also be effectively avoided by the use of bar or pat coding sequences, such as is described in U.S. Pat. No. 5,712,135. These preferred marker DNAs encode second proteins or polypeptides inhibiting or neutralizing the action of glutamine synthetase inhibitor herbicides phosphinothricin (glufosinate) and glufosinate ammonium salt (Basta®, Ignite®).

The plasmid containing one or more of these genes is introduced into either plant protoplasts or callus cells by any of the previously mentioned techniques. If the marker gene is a selectable gene, only those cells that have incorporated the DNA package survive under selection with the appropriate phytotoxic agent. Once the appropriate cells are identified and propagated, plants are regenerated. Progeny from the transformed plants must be tested to insure that the DNA package has been successfully integrated into the plant genome.

There are numerous factors that influence the success of transformation. The design and construction of the exogenous gene construct and its regulatory elements influence the integration of the exogenous sequence into the chromosomal DNA of the plant nucleus and the ability of the transgene to be expressed by the cell. A suitable method for introducing the exogenous gene construct into the plant cell nucleus in a non-lethal manner is essential. Importantly, the type of cell into which the construct is introduced must, if whole plants are to be recovered, be of a type which is amenable to regeneration, given an appropriate regeneration protocol.

Prokaryotes can also be used as host cells for the initial cloning steps of the present invention. Methods, vectors, plasmids and host cell systems are well known to the skilled artisan that can be used for these initial cloning and expansion steps and will not be described herein.

In another embodiment of the present disclosure an embryo-specific promoter, an endosperm-specific promoter, or an ear-specific promoter can be inserted so as to be operatively linked to a gene encoding a miRNA-resistant plant growth and/or development associated gene or a plant growth and/or development associated gene having one or more premature stop codons, such as REV, in the plant to be transformed using methods well known to the skilled artisan. In some embodiments, the embryo promoter is an early phase-specific embryo promoter. Insertion of the promoter will allow for the embryo-specific expression, endosperm-specific expression, or ear-specific expression of the gene, e.g., a modified REV, before or during the developing seeds of the transgenic plant. Without wishing to be bound by theory, the mRNA comprising the modified REV encoding mRNA may, for example, have a longer half-life in the plant cell resulting in a plant that produces substantially larger and/or more seeds than the wild-type plant; or alternatively, the mRNA comprising the modified REV encoding mRNA may bind miRNA in the plant cell allowing a longer half-life for endogenous wild-type REV mRNA in the plant cell and thus more REV protein resulting in a transgenic plant that produces substantially larger and more seeds than the wild-type plant. The alternative co-suppression mechanism for a modified REV activity is set forth herein in the specification.

Transgenic plants of particular interest in the methods of the present disclosure include but are not limited to monocot and dicots particularly from the families Brassicaceae (Crucifereae), Gramineae, Malvaceae, or Leguminoseae-Papilionoideae. Plants of particular interest within these families include, but are not limited to canola, corn, camelina, cotton, wheat, rice, soybean, barley and other seed producing plants, as well as other plants including, but not limited to alfalfa, sugar cane and the like, of agricultural interest which comprise in a particular embodiment of the present invention, for example, a miRNA-resistant REV transgene that has a reduced binding affinity for miRNA, or a REVstop transgene under the control of an appropriate promoter, such as an embryo-specific promoter (e.g., an early phase-specific embryo promoter), an endosperm-specific promoter, or an ear-specific promoter. The transgene can be from the same species as the transgenic plant, or the transgene can be from a heterologous plant. Of particular interest is a transgenic plant comprising the modified REV transgene from Arabidopsis, or Zea mays. The embryo-specific promoter (e.g., an early phase-specific embryo promoter), the endosperm-specific promoter, or the ear-specific promoter can also be from the same species as the transgenic plant, or from a heterologous plant. For example, the embryo-specific promoter (e.g., an early phase-specific embryo promoter), the endosperm-specific promoter, or the ear-specific promoter can be from the same plant species as the REV transgene or even from another species of plant. Of particular interest are early phase-specific embryo promoters from Arabidopsis or Lesquerella fendleri, but the early phase-specific promoter can be obtained from another species of plant. Specific combinations of early phase-specific promoter and mutated REV transgene that have been found to be suitable for the methods of the present disclosure include, but are not limited to (a) Lesquerella fendleri LFAH12 promoter/modified Arabidopsis REV; (b) Arabidopsis AAP1 promoter/modified Arabidopsis REV; (c) Arabidopsis LEC2 promoter/modified Arabidopsis REV; and (d) Arabidopsis 2S2 promoter/modified Arabidopsis REV. In some other embodiments, an endosperm-specific promoter (e.g., a legumin 1A (LEG1A) gene promoter), or an ear-specific promoter (e.g., AGAMOUS gene promoter or CLAVATA 1 gene promoter) may be used. In a particular embodiment of the present disclosure, these mutated transgene constructs have been used to transform canola, but can be used to transform other plant species. In particular, they can be used to produce transgenic plants having increased seed size and/or seed number in soybeans, corn, cotton, camelina, rice, wheat, barley, alfalfa, and other crops of agricultural interest.

The present disclosure also provides methods for selecting a growth and/or development associated gene that increases plant yield. In the method, a sequence search program can be used to search for miRNA binding sites in a gene of interest, and the selected gene of interest is mutated to encode an mRNA that does not bind miRNA, or comprises one or more early termination codons, and the mutated gene of interest is functionally associated with an appropriate promoter, such as an embryo-specific promoter (e.g., an early phase-specific embryo promoter), an endosperm-specific promoter, or an ear-specific promoter in an expression plasmid or vector. The expression plasmid or vector comprising the mutated gene of interest is transfected into a plant cell using a method known in the art to form a transgenic cell. The cell comprising the mutated transgene is grown up and regenerated into a transgenic plant by known methods, including those disclosed above until transgenic plants are obtained. The transgenic plants are observed for increased yield as compared with a wild-type plant and those growth and/or development associated genes that were used to obtain the transgenic plants with increased yield are selected for further development. Transgenic plants comprising the selected growth and/or development associated gene can be further developed to provide plants of agricultural importance with a higher yield than the wild-type plants. In some embodiments, the plant yield of the transgenic plants of the present invention calculated by per plant, or per acre increases about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 300%, about 350%, about 400%, or more compared to a control plant, such as a wild type plant or a plant comprising a control vector.

The transgenic plants of the present invention having increased seed number and/or see size, which may lead to increased yield, can be used for other purposes. For example, the transgenic plants can be subjected to breeding techniques well know in the art to create new plants through gene stacking, wherein the new plants inherit the transgenes of the present invention, with one or more other agriculturally desired traits. As used herein, “agronomically important traits” include any phenotype in a plant or plant part that is useful or advantageous for human use. Examples of agronomically important traits include but are not limited to those that result in increased biomass production, production of specific biofuels, increased food production, improved food quality, etc. Additional examples of agronomically important traits includes pest resistance, vigor, development time (time to harvest), enhanced nutrient content, novel growth patterns, flavors or colors, salt, heat, drought and cold tolerance, and the like. Agronomically important traits do not include selectable marker genes (e.g., genes encoding herbicide or antibiotic resistance used only to facilitate detection or selection of transformed cells), hormone biosynthesis genes leading to the production of a plant hormone (e.g., auxins, gibberllins, cytokinins, abscisic acid and ethylene that are used only for selection), or reporter genes (e.g. luciferase, β-glucuronidase, chloramphenicol acetyl transferase (CAT, etc.). The one or more other agriculturally desired traits can be due to natural genes, mutants, and/or transgenes.

### Breeding Methods

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection. In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enters a synthetic varies widely. In practice, numbers of parental lines range from 10 to several hundred, with 100-200 being the average. Broad based synthetics formed from 100 or more clones would be expected to be more stable during seed multiplication than narrow based synthetics.

Hybrids. A hybrid is an individual plant resulting from a cross between parents of differing genotypes. Commercial hybrids are now used extensively in many crops, including corn (maize), sorghum, sugarbeet, sunflower and broccoli. Hybrids can be fainted in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

Bulk Segregation Analysis (BSA). BSA, a.k.a. bulked segregation analysis, or bulk segregant analysis, is a method described by Michelmore et al. (Michelmore et al., 1991, Identification of markers linked to disease-resistance genes by bulked segregant analysis: a rapid method to detect markers in specific genomic regions by using segregating populations. Proceedings of the National Academy of Sciences, USA, 99:9828-9832) and Quarrie et al. (Quarrie et al., Bulk segregant analysis with molecular markers and its use for improving drought resistance in maize, 1999, Journal of Experimental Botany, 50(337):1299-1306).

For BSA of a trait of interest, parental lines with certain different phenotypes are chosen and crossed to generate F2, doubled haploid or recombinant inbred populations with QTL analysis. The population is then phenotyped to identify individual plants or lines having high or low expression of the trait. Two DNA bulks are prepared, one from the individuals having one phenotype (e.g., resistant to virus), and the other from the individuals having reversed phenotype (e.g., susceptible to virus), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs). Markers linked to the phenotype can be identified and used for breeding or QTL mapping.

It should be understood that the examples and embodiments described herein are for illustrative purposes only and that various modifications or changes in light thereof will be suggested to persons skilled in the art and are to be included within the spirit and purview of this application.

### Tissue Culture

Modern plant tissue culture is performed under aseptic conditions under filtered air. Living plant materials from the environment are naturally contaminated on their surfaces (and sometimes interiors) with microorganisms, so surface sterilization of starting materials (explants) in chemical solutions (usually alcohol or bleach) is required. Explants are then usually placed on the surface of a solid culture medium, but are sometimes placed directly into a liquid medium, particularly when cell suspension cultures are desired. Solid and liquid media are generally composed of inorganic salts plus a few organic nutrients, vitamins and plant hormones. Solid media are prepared from liquid media with the addition of a gelling agent, usually purified agar.

The composition of the medium, particularly the plant hormones and the nitrogen source (nitrate versus ammonium salts or amino acids) have profound effects on the morphology of the tissues that grow from the initial explant. For example, an excess of auxin will often result in a proliferation of roots, while an excess of cytokinin may yield shoots. A balance of both auxin and cytokinin will often produce an unorganized growth of cells, or callus, but the morphology of the outgrowth will depend on the plant species as well as the medium composition. As cultures grow, pieces are typically sliced off and transferred to new media (subcultured) to allow for growth or to alter the morphology of the culture. The skill and experience of the tissue culturist are important in judging which pieces to culture and which to discard. As shoots emerge from a culture, they may be sliced off and rooted with auxin to produce plantlets which, when mature, can be transferred to potting soil for further growth in the greenhouse as normal plants.

The tissue obtained from the plant to culture is called an explant. Based on work with certain model systems, particularly tobacco, it has often been claimed that a totipotent explant can be grown from any part of the plant. However, this concept has been vitiated in practice. In many species explants of various organs vary in their rates of growth and regeneration, while some do not grow at all. The choice of explant material also determines if the plantlets developed via tissue culture are haploid or diploid. Also the risk of microbial contamination is increased with inappropriate explants. Thus it is very important that an appropriate choice of explant be made prior to tissue culture.

The specific differences in the regeneration potential of different organs and explants have various explanations. The significant factors include differences in the stage of the cells in the cell cycle, the availability of or ability to transport endogenous growth regulators, and the metabolic capabilities of the cells. The most commonly used tissue explants are the meristematic ends of the plants like the stem tip, auxiliary bud tip and root tip. These tissues have high rates of cell division and either concentrate or produce required growth regulating substances including auxins and cytokinins. Some explants, like the root tip, are hard to isolate and are contaminated with soil microflora that become problematic during the tissue culture process. Certain soil microflora can form tight associations with the root systems, or even grow within the root. Soil particles bound to roots are difficult to remove without injury to the roots that then allows microbial attack. These associated microflora will generally overgrow the tissue culture medium before there is significant growth of plant tissue. Aerial (above soil) explants are also rich in undesirable microflora. However, they are more easily removed from the explant by gentle rinsing, and the remainder usually can be killed by surface sterilization. Most of the surface microflora do not form tight associations with the plant tissue. Such associations can usually be found by visual inspection as a mosaic, de-colorization or localized necrosis on the surface of the explant.

An alternative for obtaining uncontaminated explants is to take explants from seedlings which are aseptically grown from surface-sterilized seeds. The hard surface of the seed is less permeable to penetration of harsh surface sterilizing agents, such as hypochlorite, so the acceptable conditions of sterilization used for seeds can be much more stringent than for vegetative tissues.

Tissue cultured plants are clones, if the original mother plant used to produce the first explants is susceptible to a pathogen or environmental condition, the entire crop would be susceptible to the same problem, conversely any positive traits would remain within the line also. Plant tissue culture is used widely in plant science; it also has a number of commercial applications. Applications include:

1. Micropropagation is widely used in forestry and in floriculture. Micropropagation can also be used to conserve rare or endangered plant species.
2. A plant breeder may use tissue culture to screen cells rather than plants for advantageous characters, e.g. pathogen resistance/tolerance.
3. Large-scale growth of plant cells in liquid culture inside bioreactors as a source of secondary products, like recombinant proteins used as biopharmaceuticals.
4. To cross distantly related species by protoplast fusion and regeneration of the novel hybrid.
5. To cross-pollinate distantly related species and then tissue culture the resulting embryo which would otherwise normally die (Embryo Rescue).
6. For production of doubled monoploid (dihaploid) plants from haploid cultures to achieve homozygous lines more rapidly in breeding programs, usually by treatment with colchicine which causes doubling of the chromosome number.
7. As a tissue for transformation, followed by either short-term testing of genetic constructs or regeneration of transgenic plants.
8. Certain techniques such as meristem tip culture can be used to produce clean plant material from virused stock, such as potatoes and many species of soft fruit.
9. Micropropagation using meristem and shoot culture to produce large numbers of identical individuals.

The following examples are provided merely as illustrative of various aspects of the present disclosure and shall not be construed to limit the methods or materials used therein in any way.

While not wishing to be bound by a particular theory, the modified plant growth and/or development nucleic acids/genes of the present invention have a longer half-life in the plant cell resulting in a plant that produces substantially larger and/or more seeds than the wild-type plant. This mechanism is consistent with the expression of a REV miRNA binding mutant transgene in a plant. The miRNA-resistant transgene does not bind the endogenous plant miRNAs, so the transgene expression is not suppressed by miRNA regulation. This results in more mutant transgene being transcribed and translated and thus more REV protein to bring about the seed yield and/or size increase. Alternatively, the modified plant growth and/or development gene encodes a mRNA that may bind miRNA in the plant cell, allowing a longer half-life for endogenous wild-type plant growth and/or development gene mRNA in the plant cell and thus more plant growth and/or development protein is available in the plant so that it produces substantially larger and more seeds than the wild-type plant. This mechanism is consistent with the expression of a REV transgene containing one or more stop codons. The REVstop transgene still has an intact miRNA binding site, so the transgene acts as a sink for binding of endogenous plant miRNAs. This sponge effect of the REVstop transgene allows greater transcription and translation of the endogenous wild type REV, leading to increased seed yield and/or seed size. However, it cannot be ruled out that the REVstop mRNA is seen as an abnormal species and causes co-suppression of the endogenous wild type REV, which may lead to increased seed yield and/or seed size.

### EXAMPLES

The following example describes the construction of an expression vector comprising an appropriate promoter and a modified gene with a role in plant growth and/or development. In particular, in some embodiments, the embryo specific promoter Lesquerella fendleri LFAH12 was operatively associated with the Arabidopsis REVOLUTA (REV) coding region (cds) that contained two premature stop codons at amino acid positions 11 and 18 designated REVstop, or associated with the Arabidopsis REVOLUTA (REV) coding region (cds) that comprises one or more mutations at a miRNA binding site. This construct was used to produce transgenic canola plants or corn plants. In some other embodiments, an endosperm-specific promoter, or an ear-specific promoter is used.

### Example 1

### Transgenic Canola Plants Expressing a Transgene Construct Designed to Confer Embryo-Specific Expression of a Revoluta Coding Region Containing a Mutated miRNA Binding Site

The following example describes the construction of an expression vector comprising an early phase embryo-specific promoter and a gene with a role in plant growth and/or development. In particular, the embryo specific promoter Lesquerella fendleri LFAH12 was operatively associated with the Arabidopsis REVOLUTA (REV) coding region (cds) that contained two nucleotide changes in the microRNA (miRNA) binding site. This construct was used to produce transgenic canola plants.

Embryo-specific over-expression of the Arabidopsis REV gene in transgenic B. napus (canola) plants resulted in increased seed yield relative to null sibling canola plants in replicated field trials across multiple locations and multiple years (WO 2007/079353, incorporated herein by reference). To determine whether the REV transgene functions at the RNA or protein level to effect the seed yield increase, a mutant REV transgene was created containing two nucleotide changes in the miRNA binding site. Mutations in the miRNA binding site of the transgene would be predicted to prevent degradation of transgene REV RNA because the binding of endogenous canola miRNA to this site has been disrupted by the mutations. If the seed yield increase were due to more REV protein expression from the transgene, the REV miRNA mutant transgene should lead to even greater production of REV protein and thus to greater seed yield.

One promoter that confers embryo-specific expression was selected for use in an expression construct designed to give transgenic expression of the REV miRNA mutant coding sequence in canola embryos (B. napus) during early embryo development. The LFAH12 promoter (oleate 12-hydroxylase:desaturase gene from Lesquerella fendleri)(Broun et al., Plant J. 13:201-210, 1998, U.S. Pat. No. 5,965,793, each incorporated herein by reference) was selected and operatively associated with the coding sequence of Arabidopsis REV having a mutation in the miRNA binding site as described below.

Construction of LFAH12 Promoter-At Rev cds miRNA Mutant-Rev 3′ UTR (TG42)

At REV cds (SEQ ID NO: 8) in plasmid pTG230 was subjected to site-directed mutagenesis to create two mutations in the microRNA binding region. The mutations in the mutated REV (SEQ ID NO: 9) created a T to A change at nucleotide 567 and a G to A change at nucleotide 570 in the Arabidopsis Revoluta coding sequence; these changes did not affect the amino acid sequence. The presence of these two mutations was verified by sequencing. The resulting At REV miRNA mutant in the vector pCR-Blunt (Invitrogen) was designated plasmid pTG509. The At REV 3′ UTR (SEQ ID NO: 15) was excised from the plasmid designated pTG234 with EcoRV and NotI and cloned into plasmid pTG509 at the same sites to give the plasmid designated pTG518. The At REV cds miRNA mutant-rev 3′ UTR cassette was taken as a SpeI-KpnI fragment from plasmid pTG518 and, along with the LFAH12 promoter (SEQ ID NO: 14) KpnI-SpeI fragment from plasmid pTG143), were ligated into pCGN1547 binary vector (McBride et al., Plant Mol. Biol. 14:269-276, 1990) that had been cut with KpnI in a three-way ligation to create LFAH12 promoter-At REV cds miRNA mutant-rev 3′ UTR in a head-to-tail orientation with the plant NPTII marker cassette, giving the plasmid designated pTG520, which has also been designated TG42.

Canola (Brassica napus) Transformation

The double haploid canola variety DH12075 was transformed with the REV miRNA mutant transgene expression construct using an Agrobacterium-mediated transformation method based on that of Maloney et al. (Plant Cell Reports 8:238, 1989).

Sterilized seeds were germinated on ½ MS (Murashige & Skoog) media with 1% sucrose in 15×60 mm Petri dishes for 5 days with approximately 40 to about 60 seeds per plate. A total of approximately 1500 seeds were germinated for the transformation construct. Seeds were not fully submerged in the germination medium. Germinated seedlings were grown at 25° C., on a 16 hour light/8 hour dark cycle.

Cotyledons were cut just above the apical meristem without obtaining any of the meristem tissue. This was done by gently gripping the two petioles with forceps immediately above the apical meristem region. Care was taken not to crush the petioles with the forceps. Using the tips of the forceps as a guide, petioles were cut using a scalpel with a sharp NO. 12 blade. Cotyledons were released onto a 15×100 mm plate of co-cultivation medium. Properly cut cotyledons separate easily. If they did not, there was a very good chance that meristematic tissue had been obtained and such cotyledons were not used. Each plate held approximately 20 cotyledons. Cotyledon explants were inoculated with Agrobacterium after every few plates were prepared to avoid wilting, which would have a negative impact on following stages of the protocol.

The REV miRNA mutant construct was introduced into Agrobacterium tumefaciens by electroporation. Agrobacterium harboring the REV miRNA mutant construct was grown in AB medium with appropriate antibiotics for two days shaking at 28° C. To inoculate cotyledon explants, a small volume of Agrobacterium culture was added to a 10×35 mm Petri dish. The petiole of each explant was dipped into the Agrobacterium culture and the cut end placed into co-cultivation medium in a Petri dish. The plates were sealed and cultured at 25° C., 16 hour light/8 hour dark for 3 days.

After 3 days, explants were transferred in sets of ten to fresh 25×100 mm Petri dishes containing shoot induction medium. This medium contained a selection agent (20 mg/l Kanamycin) and hormone (4.5 mg/l brassinosteroid (BA)). Only healthy-looking explants were transferred. Explants were kept on shoot induction medium for 14 to 21 days. At this time, green calli and possibly some shoot development and some non-transformed shoots could be observed. Non-transformed shoots were easily recognized by their white and purple color. Kanamycin-sensitive shoots were removed by cutting them away and all healthy-looking calli were transferred to fresh plates of shoot induction medium. The explants were kept on these plates for another 14 to 21 days.

After 2 to 3 weeks, shoots that were dark green in color were transferred to plates containing shoot elongation medium. This medium contained a selection agent (20 mg/l Kanamycin) but did not contain any hormones. Five shoots were transferred to each plate. The plates were sealed and returned to the tissue culture room. Transformed shoots that appeared vitrious were transferred to shoot elongation medium containing phloroglucinol (150 mg/l). Shoots that became healthy and green were returned to shoot elongation medium plates. Repeated transfers of vitrious shoots to fresh plates of the same medium were required in some cases to obtain normal looking shoots.

Shoots with normal morphology were transferred to 4 oz. jars with rooting medium containing 0.5 mg/l indole butyric acid. Any excess callus was cut away when transferring shoots to the jars. Shoots could be maintained in jars indefinitely by transferring them to fresh jars containing 0.2 mg/l indole butyric acid approximately every 6 weeks.

Once a good root system had formed, the T₀ generation shoots were removed from jars, agar removed from the roots, and the plantlet transferred to potting soil. Each independent T₀ plantlet represented an independent occurrence of insertion of the transgene into the canola genome and was referred to as an event. A transparent cup was placed over the plantlet for a few days, allowing the plant to acclimatize to the new environment. Once the plant had hardened, the cup was removed. The T₀ transgenic events were then grown to maturity and T₁ seeds collected.

### T₀ Event Characterization

The number of transgene insertion site loci was determined in each event by Southern analysis. REV miRNA mutant transgene expression in the T₀ events was measured by real-time PCR. REV expression data were obtained for a single time point in embryo development, 19 days after pollination (DAP). From these data it was concluded that, at this developmental time point, the LFAH12 promoter was driving REV miRNA mutant RNA production.

T₀ plants were successfully generated for the REV miRNA mutant construct.

### Example 2

In this example the transgenic canola plants comprising the Arabidopsis REV miRNA mutant transgene under the control of the embryo-specific LFAH12 promoter were tested in field trials.

Advancement of Transgenic REV miRNA Mutant Events to Field Trials

T₀ events were selected for advancement to field trials based on a combination of transgene expression and transgene insertion locus number. Events with verified transgene expression and a single transgene insertion locus were assigned the highest priority to be carried forward to field testing. In some instances, events with multiple insertion loci were selected if the presence of multiple genes gave a high overall transgene expression level due to gene dosage.

T₁ seeds from selected events were grown as segregating T₁ populations in field plots. Each event was planted as a two row, twenty four plant plot. For events with a single transgene insertion locus, segregation of the transgene among the twenty four T₁ plants would produce a distribution of approximately six null plants lacking the transgene, twelve heterozygous plants, and six homozygous plants. Each T₁ plant was individually bagged before flowering to prevent out-crossing. T₂ seeds from each of the twenty four T₁ plants were harvested separately.

The T₂ seed stocks were used to identify which of the twenty four parent T₁ plants were null, heterozygous, or homozygous. Approximately thirty T₂ seeds from each T₁ plant were germinated on filter paper in petri dishes with a solution containing the antibiotic G418, an analog of kanamycin. Since the plants were co-transformed with the nptII resistance gene as a selectable marker, only those seeds carrying the transgene would germinate and continue to grow. If all the seeds on a plate were sensitive to G418, then the T₁ parent was identified as a null line. If all the seeds on a plate were resistant to G418, then the T₁ parent was identified as a homozygous line. If approximately one quarter of the seeds on a plate were sensitive and the rest resistant, the T₁ parent was identified as a heterozygous line. T₂ seeds from homozygous T₁ parents from the same transformation event were bulked to generate homozygous seed stocks for field trial testing. T₂ seeds from null T₁ parents from the same transformation event were bulked to generate null sibling seed stocks for field trial testing. +

### Field Trial Design

The effect of the REV miRNA mutant transgene driven by the LFAH12 embryo-specific promoter on seed yield increase and seed size was tested in transgenic canola lines by comparing each transgenic line directly with its null sibling in the field in large scale replicated trials. Since the null sibling arises from segregation of the transgene in the T₁ generation, the null and homozygous siblings are nearly identical genetically. The only significant difference is the presence or absence of the REV miRNA mutant transgene. This near genetic identity makes the null sibling the optimal control for evaluation of the effect of the REV miRNA mutant transgene. As the main objective of the trial was the comparison of the transgenic line from an event to its null segregant, a split plot design was chosen. This design gave a high level of evaluation to the interaction between the transgenic and non-transgenic subentries and the differences between transgenic subplots between events (the interaction of subplot and main plot) and a lower level of evaluation to the differences between overall events or the main plot.

Field trials were conducted at multiple locations across prairie environments to assess yield phenotypes under the range of environmental conditions in which canola is typically grown. At all locations, each transgenic event was physically paired with its null sibling in adjacent plots. Each plot pair of homozygous and null siblings was replicated four times at each trial location. The locations of the four replicate plot pairs in each trial were randomly distributed at each trial location. Plots were 1.6 m by 6 m and planted at a density of approximately 142 seeds per square meter. Plants were grown to maturity using standard agronomic practices typical of commercial production for canola.

### Example 3

In this example the seed yield of the transgenic canola that expressed the REV miRNA mutant transgene from an embryo-specific promoter in various field trials over several prairie environments was determined.

All plots at each yield field trial location were individually harvested with a combine. Total seed yield data were collected as total seed weight adjusted for moisture content from each plot. For every transgenic event in each trial, the mean of the total yield from the four replicate plots of each homozygous line was compared to the mean of the total yield from the four replicate plots of the associated null sibling line. This comparison was used to evaluate the effect of the REV miRNA mutant transgene on total seed yield. Results from each of the multiple trial locations were combined to give an across trials analysis of the effect of the REV miRNA mutant transgene on total seed yield. Statistical analysis of variance at each trial location permitted the assignment of a threshold for significance (P=0.05) for differences in total seed yield between homozygous transgenic lines and their null siblings.

Six total transgenic REV miRNA mutant canola events were tested. All 6 events represent independent random integrations in the canola genome. The relative RNA levels of the Arabidopsis REV miRNA mutant transgene was highest for one transgenic event, designated TG42-07, by real-time PCR compared to the other five events (measuring T₀ tissue). This event showed statistically significant increases in total yield across all locations (Table 5). This result demonstrated that over expression of REV miRNA mutant using an embryo-specific promoter resulted in increased seed yield.

TABLE 5
Change in total seed yield in homozygous LFAH12 promoter-At REV
miRNA mutant plants relative to their null siblings. All values are
statistically significant (P = 0.05).
Field Trial Locations

| | | | Portage | |
|---|---|---|---|---|
| | Fort Saskatchewan | MacGregor | La Prairie | Across trials |
| Event | % Yield | % Yield | % Yield | % Yield |
| TG42-07 | 42.1 | 28.0 | 36.6 | 35.3 |

### Example 4

In this example the seed yield of transgenic corn expressing a ZmREV miRNA mutant transgene from three tissue-specific promoters in multiple field trials over several environments will be determined.

Construction of ZmOLE Promoter-ZmRLD1 cds miRNA Mutant-Zm REV 3′ UTR-PINII 3′ UTR (TGZM67), ZmLEG1A Promoter-ZmRLD1 cds miRNA Mutant-Zm REV 3′ UTR-PINII 3′ UTR (TGZM66) and ZmZAG1 Promoter-ZmRLD1 cds miRNA Mutant-Zm REV 3′ UTR-PINII 3′ UTR (TGZM65)

ZmRLD1 coding sequence (ZmRLD1 cds, SEQ ID NO: 10, corresponding to GenBank AY501430, which comprising 5′ UTR, coding region, and 3′UTR of ZmRLD1, SEQ ID NO: 13) constructs driven by the embryo-specific Zm oleosin (ZmOLE) promoter (SEQ ID NO: 34), the endosperm-specific Zm legumin 1A (ZmLEG1A) promoter (SEQ ID NO: 35) and the ear-specific Zm ZAG1 promoter (SEQ ID NO: 36) were built. Zm RLD1 cds-Zm RLD1 3′ UTR in pCR Blunt was subjected to site-directed mutagenesis to create two mutations in the microRNA binding region. ZmRLD1 3′ UTR's comprises SEQ ID NO. 37. The mutations in the mutated corn REV (SEQ ID NO: 11) created a T to A change at nucleotide 579 and a G to A change at nucleotide 582 in the Zea mays rolled leaf 1 (RLD1) coding sequence; these changes did not affect the amino acid sequence. The presence of these two mutations was verified by sequencing. The resulting ZmRLD1 miRNA mutant-Zm RLD1 3′ UTR in the vector pCR-Blunt (Invitrogen) was designated plasmid pTG1091. The ZmRLD1 miRNA mutant cds-Zm RLD1 3′ UTR was excised from pTG1091 and cloned into plasmid PHP34354 to give ZmZAG1 promoter-ZmRLD1 miRNA mutant cds-Zm RLD1 3′ UTR-PINII 3′ UTR (pTG1358) or into plasmid PHP34025 to give ZmLEG1A promoter-ZmRLD1 miRNA mutant cds-Zm RLD1 3′ UTR-PINII 3′ UTR (pTG1359). To create ZmOLE promoter-ZmRLD1 miRNA mutant cds-Zm RLD1 3′ UTR-PINII 3′ UTR (pTG1366), the ZmRLD1 miRNA mutant cds-Zm RLD1 3′ UTR fragment was excised from pTG1359 and cloned into PHP34066. Finally, all 3 promoter-ZmRLD1 miRNA mutant cds-Zm RLD1 3′ UTR-PINII 3′ UTR cassettes from pTG1358, pTG1359, and pTG1366 were moved into PHP22964 containing plant selectable markers to give TGZM65 (pTG1379, ZmZAG1 promoter-ZmRLD1 cds miRNA mutant-Zm REV 3′ UTR-PINII 3′UTR), TGZM66 (pTG1380, ZmLEG1A promoter-ZmRLD1 cds miRNA mutant-Zm REV 3′ UTR-PINII 3′UTR) and TGZM67 (pTG1381, ZmOLE promoter-ZmRLD1 cds miRNA mutant-Zm REV 3′ UTR-PINII 3′UTR).

### 2010 Field Trials for Corn

For each of the three constructs, multiple expressing, single copy events were generated. Performance of hybrid events will be compared to appropriate checks with the experimental design being a randomized complete block. Events will be yield tested in multi-location, multi-replication trials in North America. Data to be collected include stand count, flowering dates (selected locations), barren count (selected locations), grain yield, and grain moisture at harvest. All data will be analyzed using a mixed model analysis.

### Example 5

In this example the seed size of transgenic canola that expressed the REV miRNA mutant transgene from an embryo-specific promoter in various field trials over several prairie environments was determined.

All plots at each field trial location were individually harvested with a combine. The LFAH12/REV miRNA event, TG42-07, showed a statistically significant increase in seed size across trials relative to null segregant siblings as measured by thousand seed weight. Results are summarized in Table 6.

TABLE 6
Change in seed size in homozygous LFAH12 promoter-At
REV miRNA mutant plants relative to their null siblings.
All values are statistically significant (P = 0.05).
% Seed Size Increase - Field Trial Locations

| | | Fort | | | Across |
|---|---|---|---|---|---|
| Event | Sakatoon | Saskatchewan | MacGregor | Portage | trials |
| TG42-07 | 6.6 | −2.2 | 2.0 | 1.7 | 2.4 |

### Example 6

### Transgenic Canola Plants Expressing A Transgene Construct Designed To Confer Embryo-Specific Expression of a REVOLUTA Translational Mutant Coding Region

Embryo-specific over-expression of the Arabidopsis REV gene in transgenic Brassica napus (canola) plants resulted in increased seed yield relative to null sibling canola plants in replicated field trials across multiple locations and multiple years (WO2007/079393, incorporated herein by reference in its entirety). A mutant REV transgene (REVstop, comprising SEQ ID NO: 42) was created containing two premature stop codons early in the coding region to determine whether any additional effect on seed size and/or number could result from expression of this mutant transgene. The presence of the premature stop codons would be predicted to prevent the REV mRNA comprising the two stop codons from being translated into a full-length REV protein.

One promoter that confers embryo-specific expression was selected for use in an expression construct designed to give transgenic expression of the REVstop translational mutant cds in canola embryos (Brassica napus) during early embryo development. In a particular embodiment, the LFAH12 (oleate 12-hydroxylase:desaturase gene from Lesquerella fendleri)(Broun et al., Plant J. 13:201-210, 1998), SEQ ID NO: 14, was used as the embryo specific promoter.

### Construction of LFAH12 Promoter-At REVstop Transgene-Rev 3′ UTR (TG45)

The Arabidopsis thaliana REV coding sequence (AT REV cds, SEQ ID NO: 8) in plasmid pTG230 was subjected to site-directed mutagenesis to create two in-frame premature stop codons early in the coding region. Mutagenesis was used to create an A to T change at nucleotide 31 and an A to T change at nucleotide 52 in the Arabidopsis REVOLUTA coding sequence, resulting in the change of the arginine at position 11 and the arginine at position 18 to stop codons. The presence of these stop codons was verified by sequencing. The resulting At REV cds with premature stop codons (REVstop transgene) in plasmid pCR-Blunt was designated plasmid pTG480. The At REV 3′ UTR (SEQ ID NO: 15) was excised from plasmid pTG234 with the restriction enzymes EcoRV and NotI and cloned into plasmid pTG480 at the same sites to give plasmid pTG496. The At REVstop-rev 3′ UTR cassette was taken as a SpeI-KpnI fragment from plasmid pTG496 and, along with the LFAH12 promoter (KpnI-SpeI fragment from plasmid pTG143), were ligated into the pCGN1547 binary vector (McBride et al., Plant Mol. Biol. 14:269-276, 1990) that had been cut with KpnI in a three-way ligation to create LFAH12 promoter-At REVstop-rev 3′ UTR in a head-to-tail orientation with the plant NPTII marker cassette, giving plasmid pTG505, which was designated TG45.

Canola (Brassica napus) Transformation

The double haploid canola variety DH12075 was transformed with the REVstop transgene expression construct using an Agrobacterium-mediated transformation method based on that of Maloney et al. (Plant Cell Reports 8:238, 1989).

Sterilized seeds were germinated on ½ MS (Murashige & Skoog) media with 1% sucrose in 15×60 mm Petri dishes for 5 days with approximately 40 to about 60 seeds per plate. A total of approximately 1500 seeds were germinated for the transformation construct. Seeds were not fully submerged in the germination medium. Germinated seedlings were grown at 25° C., on a 16 hour light/8 hour dark cycle.

Cotyledons were cut just above the apical meristem without obtaining any of the meristem tissue. This was done by gently gripping the two petioles immediately above the apical meristem region. Care was taken not to crush the petioles. The petioles were cut using a sharp scalpel blade. Cotyledons were released onto a 15 mm×100 mm plate of co-cultivation medium. Properly cut cotyledons separated easily. If they did not, there was a very good chance that meristem tissue had been obtained and such cotyledons were not used. Each plate held approximately 20 cotyledons. Cotyledon explants were inoculated with Agrobacterium after every few plates were prepared to avoid wilting, which would have a negative impact on following stages of the protocol.

The REVstop construct was introduced into Agrobacterium tumefaciens by electroporation. Agrobacterium harboring the REVstop construct was grown in AB medium with appropriate antibiotics for two days shaking at 28° C. To inoculate cotyledon explants, a small volume of Agrobacterium culture was added to a 10 mm×35 mm Petri dish. The petiole of each explant was dipped into the Agrobacterium culture and the cut end placed into co-cultivation medium in a Petri dish. The plates were sealed and cultured at 25° C., 16 hour light/8 hour dark, for 3 days.

After 3 days, explants were transferred in sets of ten to fresh 25 mm×100 mm Petri dishes containing shoot induction medium. This medium contained a selection agent (20 mg/l Kanamycin) and hormone (4.5 mg/l brassinosteroid (BA)). Only healthy-looking explants were transferred. Explants were kept on shoot induction medium for 14 to 21 days. At this time, green calli and possibly some shoot development and some non-transformed shoots were observed. Non-transformed shoots were easily recognized by their white and purple color. Kanamycin-sensitive shoots were removed by cutting them away and all healthy-looking calli were transferred to fresh plates of shoot induction medium. The explants were kept on these plates for another 14 to 21 days.

After 2 to 3 weeks, shoots that were dark green in color were transferred to plates containing shoot elongation medium. This medium contained a selection agent (20 mg/l Kanamycin) but did not contain any hormones. Five shoots were transferred to each plate. The plates were sealed and tissue culture was continued. Transformed shoots that appeared vitrious were transferred to shoot elongation medium containing phloroglucinol (150 mg/l). Shoots that became healthy and green were returned to shoot elongation medium plates. Repeated transfers of vitrious shoots to fresh plates of the same medium were required in some cases to obtain normal looking shoots.

Shoots with normal morphology were transferred to 4 oz. jars with rooting medium containing 0.5 mg/l indole butyric acid. Any excess callus was cut away when transferring shoots to the jars. Shoots could be maintained in jars indefinitely by transferring them to fresh jars containing 0.2 mg/l indole butyric acid approximately every 6 weeks.

Once a good root system had formed, the T₀ generation shoots were removed from the jars, agar removed from the roots, and the plantlet transferred to potting soil. Each independent T₀ plantlet represented an independent occurrence of insertion of the transgene into the canola genome and was referred to as an event. A transparent cup was placed over the plantlet for a few days, allowing the plant to acclimatize to the new environment. Once the plant had hardened, the cup was removed. The T₀ transgenic events were then grown to maturity in the greenhouse and T₁ seeds collected.

### T₀ Event Characterization

The number of transgene insertion site loci was determined in each event by Southern analysis. REVstop expression in the T₀ events was measured by real-time PCR. REV expression data were obtained for a single time point in embryo development, 19 days after pollination (DAP). From these data it was concluded that, at this developmental time point, the LFAH12 promoter was driving REVstop mRNA production.

T₀ plants were successfully generated for the REVstop construct.

### Example 7

### Evaluation of the Effect of REV Translational Mutant Transgene Expression During Embryo Development on Canola Yield in Replicated Field Trials

In this example the transgenic canola plants comprising the Arabidopsis REVstop transgene under the control of the embryo-specific LFAH12 promoter were tested in field trials.

### Advancement of Transgenic REV Translational Mutant Events to Field Trials.

T₀ events were selected for advancement to field trials based on a combination of REVstop transgene expression and REVstop transgene insertion locus number. Events with verified REVstop transgene expression and a single transgene insertion locus were assigned the highest priority to be carried forward to field testing. In some instances, events with multiple insertion loci were selected if the presence of multiple genes gave a high overall transgene expression level due to gene dosage.

T₁ seeds from selected events were grown as segregating T₁ populations in field plots. Each event was planted as a two row, twenty four plant plot. For events with a single transgene insertion locus, segregation of the transgene among the twenty four T₁ plants would produce a distribution of approximately six null plants lacking the transgene, twelve heterozygous plants, and six homozygous plants. Each T₁ plant was individually bagged before flowering to prevent out-crossing. T₂ seeds from each of the twenty four T₁ plants were harvested separately.

The T₂ seed stocks were used to identify which of the twenty four parent T₁ plants were null, heterozygous, or homozygous. Approximately thirty T₂ seeds from each T₁ plant were germinated on filter paper in Petri dishes with a solution containing the antibiotic G418, an analog of kanamycin. Since the plants were co-transformed with the nptII resistance gene as a selectable marker, only those seeds carrying the transgene would germinate and continue to grow. If all the seeds on a plate were sensitive to G418, then the T₁ parent was identified as a null line. If all the seeds on a plate were resistant to G418, then the T₁ parent was identified as a homozygous line. If approximately one quarter of the seeds on a plate were sensitive and the rest resistant, the T₁ parent was identified as a heterozygous line. T₂ seeds from homozygous T₁ parents from the same transformation event were bulked to generate homozygous seed stocks for field trial testing. T₂ seeds from null T₁ parents from the same transformation event were bulked to generate null sibling seed stocks for field trial testing.

### Field Trial Design

The effect of the REVstop transgene driven by the LFAH12 embryo-specific promoter on seed yield increase and seed size was tested in transgenic canola lines by comparing each transgenic line directly with its null sibling in the field in large scale replicated trials. Since the null sibling arises from segregation of the transgene in the T₁ generation, the null and homozygous siblings are nearly identical genetically. The only significant difference is the presence or absence of the REVstop transgene. This near genetic identity makes the null sibling the optimal control for evaluation of the effect of the REVstop transgene. As the main objective of the trial was the comparison of the transgenic line from an event to its null segregant, a split plot design was chosen. This design gives a high level of evaluation to the interaction between the transgenic and non-transgenic subentries and the differences between transgenic subplots between events (the interaction of subplot and main plot) and a lower level of evaluation to the differences between overall events or the main plot.

Field trials were conducted at multiple locations across prairie environments to assess yield phenotypes under the range of environmental conditions in which canola is typically grown. At all locations, each transgenic event was physically paired with its null sibling in adjacent plots. Each plot pair of homozygous and null siblings was replicated four times at each trial location. The locations of the four replicate plot pairs in each trial were randomly distributed at each trial location. Plots were 1.6 m by 6 m and planted at a density of approximately 142 seeds per square meter. Plants were grown to maturity using standard agronomic practices typical of commercial production of canola.

### Example 8

### Expression of a REV Translational Mutant Transgene from an Embryo-Specific Promoter to Increase Seed Yield in Transgenic Canola

All plots at each yield field trial location were individually harvested with a combine. Total seed yield data were collected as total seed weight adjusted for moisture content from each plot. For every transgenic event in each trial, the mean of the total yield from the four replicate plots of each homozygous line was compared to the mean of the total yield from the four replicate plots of the associated null sibling line. This comparison was used to evaluate the effect of the REVstop transgene on total seed yield. Results from each of the multiple trial locations were combined to give an across trials analysis of the effect of the REVstop transgene on total seed yield. Statistical analysis of variance at each trial location permitted the assignment of a threshold for significance (P=0.05) for differences in total seed yield between homozygous transgenic lines and their null siblings.

The transgenic REVstop canola lines that showed a statistically significant increase in total seed yield at various locations as summarized in Table 7. Eight total events were tested. One transgenic event, TG45-23, showed statistically significant increases in total yield across all locations. Another transgenic event, TG45-24, showed statistically significant increases in total yield at 2 out of 3 locations and an overall positive yield increase across all locations. These results demonstrate that over expression of the REVstop transgene using an embryo-specific promoter results in increased seed yield.

TABLE 7
Change in total seed yield in homozygous LFAH12 promoter-At
REVstop plants relative to their null siblings. All values are
statistically significant (P = 0.05) except where denoted.

| | Field Trial Locations | | | |
|---|---|---|---|---|
| | Fort | | Portage La | |
| | Saskatchewan | MacGregor | Prairie | Across trials |
| Event | % yield | % yield | % Yield | % Yield |
| TG45-23 | 14.1 | 27.5 | 44.0 | 27.1 |
| TG45-24 | −3.8* | 14.9 | 13.5 | 7.2* |

*not statistically significant

### Example 9

### Transgenic Soybean Plants Expressing A Transgene Construct Designed To Confer Embryo-Specific Expression of a REVOLUTA Translational Mutant Coding Region

Due to the seed yield increase that was seen for transgenic canola lines expressing the REVstop transgene, a construct was built for soybean to determine if the seed yield increase could be reproduced for another dicotyledonous crop.

One promoter that confers embryo-specific expression was selected for use in an expression construct designed to give transgenic expression of the At REVstop translational mutant cds in soybean embryos (Glycine max) during early embryo development. In a particular embodiment, the LEC2 (leafy cotyledon 2 gene from Arabidopsis) (Kroj et al., Development 130:6065-6073, 2003), SEQ ID NO: 16, was used as the embryo specific promoter.

### Construction of LEC2 Promoter-At REVstop Transgene-Rev 3′ UTR (TGGM24)

The Arabidopsis LEC2 promoter was amplified from Arabidopsis ecotype Columbia genomic DNA with primers KpnLec2pr586F (5′GGTACCTGTCCATCAACCCATGCCTC 3′, SEQ ID NO: 43) and Lec2-94R (5′CTGTTGTGAAGTGCGAGCGATTGT 3′, SEQ ID NO: 44) and digested with BglII. The resulting LEC2 promoter PCR fragment was then cloned into pBluescript that had been digested with EcoRV and BamHI to give pTG742. The LEC2 promoter was then taken from pTG742 and inserted into pCR-blunt to give pTG1006. The At REVstop-rev 3′ UTR cassette was taken as an Asp718 fragment from plasmid pTG496 (see Example 1) and cloned into pTG1006, which had been digested with Asp718. The resulting plasmid, pTG1029, was LEC2 promoter-At REVstop-rev 3′ UTR in pCR-Blunt, which was designated TGGM24.

### Soybean Transformation and Field Trials

Soybean transformation and event selection were carried out as described in WO2007079353, which is incorporated by reference in its entirety.

Two LEC2-At REVstop events (TGGM24-X4 and TGGM24-X31) were tested the first year in replicated field trials at 3-4 locations. Each single T3 line homozygous for the transgene was put in a split plot with a bulked null control and the split plot was replicated 4 times at each location. One of the events was represented by 2 distinct homozygous T3 lines: TGGM24-X4-7H and TGGM24-X4-15H. The null lines that were bulked to serve as control for TGGM24-X4-7H and TGGM24-X4-15H were TGGM24-X4-8, 9, and 14. The null lines that were bulked to serve as control for TGGM24-X31-10H were TGGM24-X31-3, 5, 13 and 14. TGGM24-X4-7H was tested at Listowel2, Ontario, Canada; St. Marc, Quebec, Canada; and Ward, N. Dak., USA. TGGM24-X4-15H was tested at Listowell and Walton, Ontario, Canada; St. Marc, Quebec, Canada; and Ward, N. Dak., USA. TGGM24-X31-10H was tested at Listowell and Walton, Ontario, Canada; St. Marc, Quebec, Canada; and Ward, N. Dak., USA.

The performance of the transgenic homozygous REVstop soybean lines in total seed yield compared to their respective bulked null controls are summarized in Table 8. One homozygous T3 line, TGGM24-X4-7H, showed statistically significant increases in total yield across all locations. The other 2 homozygous T3 REVstop lines did not show seed yield increase across trial sites. These results demonstrate that over expression of the REVstop transgene using an embryo-specific promoter can result in increased seed yield also in soybean.

TABLE 8
Change in total seed yield in homozygous LEC2 promoter-At REVstop
soybean lines relative to their bulked null sibling lines.

| | Field Trial Locations | | | | | |
|---|---|---|---|---|---|---|
| | | | | | | Across |
| | Listowel1 | Listowel2 | St Marc | Ward | Walton | trials |
| Event | % yield | % yield | % Yield | % Yield | % Yield | % Yield |
| TGGM24- | NT | 90.2 | 71.7 | 30.9 | NT | 64.3 |
| X4-7H | | | | | | |
| TGGM24- | −2.7* | NT | 6.9 | 3.4* | −17.0 | −2.4* |
| X4-15H | | | | | | |
| TGGM24- | −5.2* | NT | −2.8* | −20.4 | −2.0* | −7.1* |
| X31-10H | | | | | | |

All values are statistically significant (P > 0.10) except where denoted.
NT = not tested at this location
*not statistically significant

### Example 10

### Second Year Field Trials with Soybean Expressing LEC2 Promoter-at REVstop Transgene

Three LEC2-At REVstop events (TGGM24-X3, TGGM24-X4 and TGGM24-X25) will be tested in the second year in replicated field trials at 2-4 locations. Each single T3 line homozygous for the transgene will be put in a split plot with a bulked null control and the split plot will be replicated 4 times at each location. The null lines that will be bulked to serve as control for TGGM24-X3-6H and TGGM24-X3-11H will be TGGM24-X3-12, 13, and 14. The null lines that will be bulked to serve as control for TGGM24-X4-7H will be TGGM24-X4-8, 9, and 14. The null lines that will be bulked to serve as control for TGGM24-X25-12H, TGGM24-X25-13H and TGGM24-X25-14H will be TGGM24-X25-1, 3, and 8. TGGM24-X3-6H will be tested at Centralia, Listowel, and Tavistock, Ontario, Canada; and St. Marc2, Quebec, Canada. TGGM24-X3-11H will be tested at Listowel, Ontario, Canada and St. Marc, Quebec, Canada. TGGM24-X4-7H will be tested at Centralia, Listowel, and Tavistock, Ontario, Canada; and St. Marc and St. Marc2, Quebec, Canada. TGGM24-X25-12H will be tested at Centralia, Ontario, Canada and St. Marc, Quebec, Canada. TGGM24-X25-13H will be tested at Listowel and Tavistock, Ontario, Canada; and St. Marc and St. Marc2, Quebec, Canada. TGGM24-X25-14H will be tested at Centralia and Tavistock, Ontario, Canada and St. Marc2, Quebec, Canada.

The performance of the transgenic homozygous REVstop soybean lines in total seed yield and thousand seed weight will be compared to their respective bulked null controls.

### Example 11

In this example the seed size of transgenic canola that expressed the REVstop transgene from an embryo-specific promoter in various field trials over several prairie environments was determined.

All plots at each field trial location were individually harvested with a combine. The LFAH12/REV miRNA events, TG45-20 and TG45-23, showed a statistically significant increase in seed size across trials relative to null segregant siblings as measured by thousand seed weight. Results are summarized in Table 9.

TABLE 9
Change in seed size in homozygous LFAH12 promoter-At
REVstop mutant plants relative to their null siblings. All values
are statistically significant (P = 0.05).
% Seed Size Increase - Field Trial Locations

| | | Fort | | | Across |
|---|---|---|---|---|---|
| Event | Sakatoon | Saskatchewan | MacGregor | Portage | trials |
| TG45-20 | 3.2 | 1.1 | 9.4 | −1.4 | 2.7 |
| TG45-23 | 4.8 | −1.1 | 2.0 | 4.1 | 3.0 |

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A transgenic plant comprising a plant growth and/or development gene having a mutated miRNA binding site or one or more early stop codons, wherein the plant growth and/or development gene is operatively associated with an embryo-specific promoter, an endosperm-specific promoter, or an ear-specific promoter and optionally a polyA sequence, and wherein the transgenic plant demonstrates an increase in yield, seed number and/or size as compared with a wild-type plant which does not comprise the mutated plant growth and/or development gene.

2. The transgenic plant according to claim 1, wherein the plant growth and/or development gene is a HD-Zip transcription factor, a NAC-containing transcription factor, a BHLH transcription factor, a MYB transcription factor, an APETALA2-like transcription factor, a SBP-like transcription factor, a SCL transcription factor, an ARF transcription factor, or an F-box protein.

3. The transgenic plant according to claim 2, wherein the HD-Zip transcription factor is the REVOLUTA (REV) gene, PHABULOSA (PHB), PHAVOLUTA (PHV), ATHB8, or ATHB15, the NAC-containing transcription factor is NAC1, CUC1, or CUC2, the BHLH transcription factor is TCP2, TCP3, TCP4, TCP10, or TCP24, the MYB transcription factor is MYB33, MYB65, or GAMYB, the APETALA2-like transcription factor is AP2, TOE1, TOE2, TOE3, or GL15, the SBP-like transcription factor is SPL3, SPL4, or SPL5, the SCL transcription factor is SCL6-II, or SCL6-III, the ARF transcription factor is ARF6, ARF10, ARF16, ARF17, or ARF18, or the F-box protein is TIR1.

4. The transgenic plant according to claim 3, wherein the REV gene is from Arabidopsis thaliana or Zea mays, Brassica napus, camelina, soybean, rice, sorghum, or wheat.

5. The transgenic plant according to claim 1, wherein the embryo-specific promoter, the endosperm-specific promoter, or the ear-specific promoter is heterologous to the plant.

6. The transgenic plant according to claim 1, wherein the embryo-specific promoter, the endosperm-specific promoter, or the ear-specific promoter is homologous to the plant.

7. The transgenic plant according to claim 1, wherein the embryo-specific promoter is an early phase-specific promoter associated with an amino acid permease gene (AAP1), an oleate 12-hydroxylase:desaturase gene, a 2S2 albumin gene (2S2), a fatty acid elongase gene (FAE1), a leafy cotyledon 2 gene (LEC2), a leafy cotyledon 1 gene (LEC1), an aspartic protease gene (ASP) or an oleosin gene, and wherein the endosperm-specific promoter is the promoter associated with a legumin 1A (LEG1A) gene, and wherein the ear-specific promoter is the promoter associated with an AGAMOUS gene or a CLAVATA 1 gene.

8. The transgenic plant according to claim 7, wherein the AAP1 promoter is the AAP1 promoter from Arabidopsis thaliana, the oleate 12-hydroxylase:desaturase promoter is the oleate 12-hydroxylase:desaturase gene promoter from Lesquerella fendleri (LFAH12), the 2S2 gene promoter is from Arabidopsis thaliana, the fatty acid elongase gene promoter is from Arabidopsis thaliana, the leafy cotyledon 2 gene promoter is from Arabidopsis thaliana, the leafy cotyledon 1 gene promoter is from Zea mays (ZmLEC1), the aspartic protease gene promoter is from Oryza sativa or Zea mays (OsASP1 or ZmASP1), the oleosin gene promoter is from Zea mays (ZmOLE), the legumin 1A (LEG1A) gene promoter is from Zea mays (ZmLEG1A), the AGAMOUS gene is from Zea mays (ZmZAG1), or the CLAVATA 1 gene promoter is from Zea mays (ZmCLV1).

9. The transgenic plant according to claim 4, wherein the Arabidopsis Revoluta coding sequence (SEQ ID NO. 8) is mutated such that a Thymidine at nucleotide 567 is changed to an Adenine and a Guanidine at nucleotide 570 is changed to an Adenine, or wherein the Zea mays REV coding sequence (Zm RLD1, SEQ ID NO. 10) is mutated such that a Thymidine at nucleotide 579 is changed to an Adenine and a Guanidine nucleotide 582 is changed to an Adenine, or wherein the Arabidopsis Revoluta coding sequence is mutated such that a stop codon is encoded at amino acid residue positions 11 and 18.

10. A transformed cell or tissue culture comprising a plant growth and/or development gene having a mutated miRNA binding site or one or more early stop codons, wherein the plant growth and/or development gene is operatively associated with an embryo-specific promoter, an endosperm-specific promoter, or an ear-specific promoter and optionally a polyA sequence, and wherein the transformed cell or tissue culture can give rise to a transgenic plant having increase in yield, seed number and/or size as compared with a wild-type plant which does not comprise the mutated plant growth and/or development gene.

11. The transformed cell or tissue culture of claim 10, wherein the plant growth and/or development gene is a HD-Zip transcription factor, a NAC-containing transcription factor, a BHLH transcription factor, a MYB transcription factor, an APETALA2-like transcription factor, a SBP-like transcription factor, a SCL transcription factor, an ARF transcription factor, an F-box protein.

12. The transformed cell or tissue culture of claim 11, wherein the HD-Zip transcription factor is the REVOLUTA (REV) gene, PHABULOSA (PHB), PHAVOLUTA (PHV), ATHB8, or ATHB15, the NAC-containing transcription factor is NAC1, CUC1, or CUC2, the BHLH transcription factor is TCP2, TCP3, TCP4, TCP10, or TCP24, the MYB transcription factor is MYB33, MYB65, or GAMYB, the APETALA2-like transcription factor is AP2, TOE1, TOE2, TOE3, or GL15, the SBP-like transcription factor is SPL3, SPL4, or SPL5, the SCL transcription factor is SCL6-II, or SCL6-III, the ARF transcription factor is ARF6, ARF10, ARF16, ARF17, or ARF18, or the F-box protein is TIR1.

13. The transgenic plant according to claim 12, wherein the REV gene is from Arabidopsis thaliana or Zea mays, Brassica napus, camelina, soybean, rice, sorghum, or wheat.

14. The transformed cell or tissue culture of claim 10, wherein the embryo-specific promoter, the endosperm-specific promoter, or the ear-specific promoter is homozygous or heterologous to the plant.

15. The transformed cell or tissue culture of claim 10, wherein the embryo-specific promoter is an early phase-specific promoter associated with an amino acid permease gene (AAP1), an oleate 12-hydroxylase:desaturase gene, a 2S2 albumin gene (2S2), a fatty acid elongase gene (FAE1), a leafy cotyledon 2 gene (LEC2), a leafy cotyledon 1 gene (LEC1), an aspartic protease gene (ASP), or an oleosin gene; and wherein the endosperm-specific promoter is the promoter associated with a legumin 1A (LEG1A) gene, and wherein the ear-specific promoter is the promoter associated with an AGAMOUS gene or a CLAVATA 1 gene.

16. The transformed cell or tissue culture of claim 15, wherein the AAP1 promoter is the AAP1 promoter from Arabidopsis thaliana, the oleate 12-hydroxylase:desaturase promoter is the oleate 12-hydroxylase:desaturase gene promoter from Lesquerella fendleri (LFAH12), the 2S2 gene promoter is from Arabidopsis thaliana, the fatty acid elongase gene promoter is from Arabidopsis thaliana, the leafy cotyledon 2 gene promoter is from Arabidopsis thaliana, the leafy cotyledon 1 gene promoter is from Zea mays (ZmLEC1), the aspartic protease gene promoter is from Oryza sativa or Zea mays (OsASP1 or ZmASP1), the oleosin gene promoter is from Zea mays (ZmOLE), the legumin 1A (LEG1A) gene promoter is from Zea mays (ZmLEG1A), the AGAMOUS gene is from Zea mays (ZmZAG1), or the CLAVATA 1 gene promoter is from Zea mays (ZmCLV1).

17. The transformed cell or tissue culture of claim 13, wherein the Arabidopsis Revoluta coding sequence (SEQ ID NO. 8) is mutated such that a Thymidine at nucleotide 567 is changed to an Adenine and a Guanidine at nucleotide 570 is changed to an Adenine, or wherein the Zea mays REV coding sequence (Zm RLD1, SEQ ID NO. 10) is mutated such that a Thymidine at nucleotide 579 is changed to an Adenine and a Guanidine nucleotide 582 is changed to an Adenine, or wherein the Arabidopsis Revoluta coding sequence is mutated such that a stop codon is encoded at amino acid residue positions 11 and 18.

18. A method for increasing seed yield and/or seed size of a plant comprising the steps of:
a) identifying at least one mutant plant growth and/or development gene comprising one or more mutations at a microRNA binding site, or one or more early stop codons;
b) constructing an expression construct comprising an embryo-specific promoter, an endosperm-specific promoter, or an ear-specific promoter operatively associated with the mutated plant growth and/or development gene;
c) transforming a plant cell with the expression vector of step (b);
d) selecting for a plant cell comprising the expression vector of step (b);
e) regenerating the plant from the plant cell comprising the expression vector of step (b); and
f) growing the plant of step (e) to obtain a mature plant with a phenotype of having an increased seed yield and/or seed size as compared with a wild-type plant which does not comprise the mutated plant growth and/or development gene(s).

19. The method according to claim 18, wherein the plant growth and/or development gene is a HD-Zip transcription factor, a NAC-containing transcription factor, a BHLH transcription factor, a MYB transcription factor, an APETALA2-like transcription factor, a SBP-like transcription factor, a SCL transcription factor, an ARF transcription factor, or an F-box protein.

20. The method according to claim 19, wherein the HD-Zip transcription factor is the REVOLUTA (REV) gene, PHB, PHV, ATHB8, or ATHB15, the NAC-containing transcription factor is NAC1, CUC1, or CUC2, the BHLH transcription factor is TCP2, TCP3, TCP4, TCP10, or TCP24, the MYB transcription factor is MYB33, MYB65, or GAMYB, the APETALA2-like transcription factor is AP2, TOE1, TOE2, TOE3, or GL15, the SBP-like transcription factor is SPL3, SPL4, or SPL5, the SCL transcription factor is SCL6-II, or SCL6-III, the ARF transcription factor is ARF6, ARF10, ARF16, ARF17, or ARF18, or the F-box protein is TIR1.

21. The method according to claim 20, wherein the REV gene is from Arabidopsis thaliana or Zea mays, Brassica napus, camelina, soybean, rice, sorghum, or wheat.

22. The method according to claim 18, wherein the embryo-specific promoter, the endosperm-specific promoter, or the ear-specific promoter is heterologous to the plant.

23. The method according to claim 18, wherein the embryo specific promoter, the endosperm specific promoter, or an ear specific promoter is homologous to the plant.

24. The method according to claim 18, wherein the embryo promoter is an early phase-specific promoter associated with an amino acid permease gene (AAP1), an oleate 12-hydroxylase:desaturase gene, a 2S2 albumin gene (2S2), a fatty acid elongase gene (FAE1), a leafy cotyledon 2 gene (LEC2), a leafy cotyledon 1 gene (LEC1), an aspartic protease gene (ASP), or an oleosin gene, and wherein the endosperm-specific promoter is the promoter associated with a legumin 1A (LEG1A) gene, and wherein the ear-specific promoter is the promoter associated with an AGAMOUS gene or a CLAVATA 1 gene.

25. The method according to claim 24, wherein the AAP1 promoter is the AAP1 promoter from Arabidopsis thaliana, the oleate 12-hydroxylase:desaturase promoter is the oleate 12-hydroxylase:desaturase gene promoter from Lesquerella fendleri (LFAH12), the 2S2 gene promoter is from Arabidopsis thaliana, the fatty acid elongase gene promoter is from Arabidopsis thaliana, the leafy cotyledon 2 gene promoter is from Arabidopsis thaliana, the leafy cotyledon 1 gene promoter is from Zea mays (ZmLEC1), the aspartic protease gene promoter is from Oryza sativa or Zea mays (OsASP1 or ZmASP1), the oleosin gene promoter is from Zea mays, the legumin 1A (LEG1A) gene promoter is from Zea mays (ZmLEG1A), the AGAMOUS gene is the ZAG1 gene from Zea mays (ZmZAG1), or the CLAVATA 1 gene promoter is from Zea mays (ZmCLV1).

26. The method according to claim 21, wherein the Arabidopsis Revoluta coding sequence (SEQ ID NO. 8) is mutated such that a Thymidine at nucleotide 567 is changed to an Adenine and a Guanidine at nucleotide 570 is changed to an Adenine, or wherein the Zea mays REV coding sequence (Zm RLD1, SEQ ID NO. 10) is mutated such that a Thymidine at nucleotide 579 is changed to an Adenine and a Guanidine nucleotide 582 is changed to an Adenine, or wherein the Arabidopsis Revoluta coding sequence is mutated such that a stop codon is encoded at amino acid residue positions 11 and 18.
